(19) United States
(12) Patent Application Publication
(10) Pub. No.: US 2025/0122503 A1
(43) Pub. Date: Apr. 17, 2025

(54) THERAPEUTIC SUPPRESSOR TRNA TO TREAT GENETIC DISEASES

(71) Applicants: The Trustees of Indiana University, Bloomington, IN (US); University of Connecticut, Farmington, CT (US)

(72) Inventors: Jeffery M. Tharp, Greenfield, IN (US); Oscar Vargas-Rodriguez, North Haven, CT (US)

(21) Appl. No.: 18/911,483

(22) Filed: Oct. 10, 2024

Related U.S. Application Data
(60) Provisional application No. 63/543,400, filed on Oct. 10, 2023.

(51) Int. Cl. C12N 15/113; A61K 31/7105

(52) U.S. Cl. CPC C12N 15/1135; A61K 31/7105; C12N 2310/14; C12N 2310/531

(57) ABSTRACT

Disclosed herein are chimeric suppressor tRNA molecules, such as tRNA molecules engineered to suppress mutations that result in premature termination codons (PTCs), e.g., by reading through such anomalous stop codons and facilitating production of full-length, functional proteins. Also disclosed are RNA polynucleotide sequences, such as for delivery to a cell, and DNA polynucleotides encoding the same, such as for engineering a cell to produce such tRNA molecules. Additionally, disclosed are methods of using such chimeric tRNA molecules, such as in a method of treating a disease or disorder characterized by the presence of a premature termination codon.

### CROSS-REFERENCE TO RELATED APPLICATIONS

This application claims priority to U.S. Provisional Application No. 63/543,400 filed Oct. 10, 2023.

### FIELD OF THE INVENTION

This disclosure generally relates to transfer RNA (tRNA), such as chimeric suppressor tRNA (sup-tRNA) molecules, which facilitate continued protein synthesis upon recognition of a stop codon. The disclosure additionally relates to polynucleotides encoding such tRNA molecules and vectors for the expression of the same in a host cell. The disclosure further relates to methods of using disclosed tRNA molecules, such as in a method of treatment.

### INCORPORATION OF SEQUENCE LISTING

The instant application contains a Sequence Listing XML which has been submitted electronically and is hereby incorporated by reference in its entirety. Said Sequence Listing XML copy, created on 2024 Oct. 8 is named IU-2023-102-02 st26.xml and is 52 KB in size.

### BACKGROUND

Premature termination codons (PTCs), which arise from point mutations that convert a canonical triplet nucleotide codon into a stop codon, play a crucial role in the development of inherited diseases and cancer. When a PTC occurs in the coding sequence of an mRNA, it leads to premature termination of protein synthesis, resulting in truncated and often non-functional proteins. Genetic diseases, e.g., cystic fibrosis and muscular dystrophy, as well as some forms of cancer, are associated with such mutations. Nonsense mutation suppression therapy has emerged as a therapeutic strategy to mitigate the effects of such disease-causing mutations. This approach typically involves using small molecules, such as readthrough agents or translational readthrough-inducing drugs, to bypass ribosomal recognition of the PTC and allow for uninterrupted mRNA translation, which can result in the production of a full-length, functional protein.

Research has shown that engineered transfer RNAs (tRNAs) having modified anticodon sequences can also recognize and “read through” PTCs, thereby effectively suppressing PTC mutations and promoting continued ribosomal translation and protein synthesis. However, therapeutic translation of engineered suppressor tRNAs faces several challenges. In one example, engineered tRNAs may not effectively recognize and suppress the various forms of PTCs, such as UAA (ochre), UAG (amber) and UGA (opal). Moreover, achieving sufficient efficacy in restoring protein production remains a challenge. Additionally, safety concerns arise from potential off-target effects, such as interference of normal cellular processes. Accordingly, there exists a need to provide engineered tRNA molecules that effectively suppress different PTCs, thereby facilitating protein production and treating a disease, or disorder, such as cancer or a genetic disorder. Furthermore, there exists a need to safely deliver suppressor tRNA without promoting toxicity or unintended physiological consequences. Aspects of the invention disclosed herein address these needs.

### INCORPORATION BY REFERENCE

Each patent, publication, and non-patent literature cited in the application is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art.

### SUMMARY OF THE INVENTION

A first aspect of the invention includes suppressor tRNA molecules (sup-tRNAs), such as sup-tRNA molecules including a mutant Methanomethylophilus alvus pyrrolysyl tRNA molecule (tRNA^{Pyl}), a human tRNA identity element, and an anticodon loop configured to base pair with a premature termination codon.

A second aspect of the invention includes polynucleotide sequences encoding the provided sup-tRNA molecules.

A third aspect of the invention includes expression systems encoding the provided sup-tRNA molecules.

A fourth aspect of the invention includes a pharmaceutical composition including a provided suppressor tRNA molecule, a provided polynucleotide sequence, or a provided expression system, or any combination thereof.

A fifth aspect of the invention includes a method of treating a genetic disease in a subject including administering to the subject a provided suppressor tRNA molecule, a provided polynucleotide sequence, or a provided expression system, or any combination thereof.

A first embodiment is a suppressor tRNA molecule including a mutant Methanomethylophilus alvus pyrrolysyl tRNA molecule (tRNA^{Pyl}), where the mutant M. alvus tRNA^{Pyl} molecule further includes: a mutated acceptor stem including a human tRNA identity element and an anticodon loop including three nucleotide residues which are complementary to a premature termination codon on an mRNA template encoding a polypeptide.

A second embodiment is a suppressor tRNA molecule, where the mutated acceptor stem includes a region of unpaired nucleotides including four nucleotides on a single strand and a region of base-paired nucleotides including fourteen paired of nucleotides, where the region of unpaired nucleotides includes at least one but less than four point mutations and the region of base-paired nucleotides includes at least one but less than seven point mutations.

A third embodiment is a suppressor tRNA molecule, where the region of unpaired nucleotides includes a guanine to adenine point mutation and the region of base-paired nucleotides includes at least one but less than four uracil to cytosine point mutation(s) and/or at least one but less than four cytosine to uracil point mutation(s).

A fourth embodiment is a suppressor tRNA molecule, where the region of unpaired nucleotides includes a single guanine to adenine point mutation, and the region of base-paired nucleotides includes one or two uracil to cytosine point mutation(s) and one cytosine to uracil point mutation.

A fifth embodiment is a suppressor tRNA molecule including the mutations C66U/U67C/G69A, C29U/C66U/U67C/G69A, C29U/U30C/C66U/U67C/G69A, U65C/C66U/U67C/G69A, C29U/U65C/C66U/U67C/G69A, or C29U/U30C/U65C/C66U/U67C/G69A with reference to SEQ ID NO:1 or SEQ ID NO:8.

A sixth embodiment is a suppressor tRNA molecule where the anticodon loop includes the nucleotide sequence: CUA, UUA or UCA.

A seventh embodiment is a suppressor tRNA molecule where the suppressor tRNA molecule is aminoacylated by a human aminoacyl-tRNA synthetase.

An eighth embodiment is a suppressor tRNA molecule where the human aminoacyl-tRNA synthetase is human alanyl-tRNA synthetase and the suppressor tRNA molecule is aminoacylated with alanine.

A ninth embodiment is a suppressor tRNA molecule including an RNA sequence having at least about 85%, 90%, 95%, 98%, or 99% sequence identity to SEQ ID NO:2, SEQ ID NO: 3, SEQ ID NO:4, SEQ ID NO:5, SEQ ID NO:6, or SEQ ID NO:7.

A tenth embodiment is a suppressor tRNA molecule including an RNA sequence having 100% sequence identity to SEQ ID NO:2, SEQ ID NO:3, SEQ ID NO:4, SEQ ID NO:5, SEQ ID NO:6, or SEQ ID NO:7.

An eleventh embodiment is a polynucleotide sequence encoding the suppressor tRNA molecule of any of the preceding embodiments.

A twelfth embodiment is an expression system including an expression cassette including a polynucleotide sequence encoding the suppressor tRNA molecule of any of the preceding embodiments.

A thirteenth embodiment is an expression system including an expression cassette including a polynucleotide sequence encoding a suppressor tRNA molecule, where the polynucleotide sequence encoding the suppressor tRNA molecule includes any one of SEQ ID NO: 9, SEQ ID NO: 10, SEQ ID NO:11, SEQ ID NO: 12, SEQ ID NO:13, and SEQ ID NO:14, or the complement thereof, or a polynucleotide sequence having at least about 85%, 90%, 95%, 98%, or 99% sequence identity to SEQ ID NO: 9, SEQ ID NO:10, SEQ ID NO:11, SEQ ID NO: 12, SEQ ID NO:13, and SEQ ID NO:14, or the complement thereof, and where the polynucleotide sequence encoding the suppressor tRNA molecule is operably linked to a promoter.

A fourteenth embodiment is an expression system where the expression cassette is stably integrated into the genome of a host cell.

A fifteenth embodiment is an expression system where the polynucleotide sequence encoding the suppressor tRNA molecule consists of a polynucleotide sequence having 100% sequence identity to any one of SEQ ID NO: 9, SEQ ID NO:10, SEQ ID NO:11, SEQ ID NO:12, SEQ ID NO:13, and SEQ ID NO:14, or the complement thereof.

A sixteenth embodiment is an expression system where the expression cassette includes at least one copy of the polynucleotide sequence encoding the suppressor tRNA molecule.

A seventeenth embodiment is an expression system where the expression cassette includes multiple copies of the polynucleotide sequence encoding the suppressor tRNA molecule, where the multiple copies of the polynucleotide sequence encoding the suppressor tRNA molecule are repeated in tandem.

An eighteenth embodiment is an expression system where the multiple copies of the polynucleotide sequence encoding the suppressor tRNA molecule repeated in tandem are each operably linked to a promoter.

A nineteenth embodiment is an expression system where the expression cassette includes a total of one, two, three, four, five, six, or seven copies of the polynucleotide sequence encoding the suppressor tRNA molecule.

A twentieth embodiment is an expression system where the promoter is an RNA polymerase type III promoter or a derivative thereof.

A twenty-first embodiment is an expression system where the RNA polymerase type III promoter or the derivative thereof is of mammalian origin.

A twenty-second embodiment is an expression system where the RNA polymerase type III promoter is a U6 promoter, H1 promoter, 7SK promoter, 7SL promoter, Y3 promoter, 5S rRNA promoter, Ad2 VAI, VAII promoter, or a derivative thereof.

A twenty-third embodiment is an expression system where the RNA polymerase type III promoter is a human U6 promoter, a human H1 promoter, a human 7SK promoter, or a derivative thereof.

A twenty-fourth embodiment is an expression system where the RNA polymerase type III promoter includes a polynucleotide sequence having at least about 80%, 85%, 90%, 95%, 98%, 99% sequence identity to SEQ ID NO:15, SEQ ID NO:16, or SEQ ID NO:17.

A twenty-fifth embodiment is an expression system where the RNA polymerase type III promoter includes or consists of a polynucleotide sequence having 100% identity to SEQ ID NO: 15, SEQ ID NO:16, or SEQ ID NO:17.

A twenty-sixth embodiment is an expression system where the expression cassette further includes a terminator sequence, where the terminator sequence flanks the 3′ end of the polynucleotide sequence encoding the suppressor tRNA molecule.

A twenty-seventh embodiment is an expression system where the terminator sequence includes a nucleotide sequence set forth in SEQ ID NO:18, SEQ ID NO:19, SEQ ID NO:20, SEQ ID NO:21, SEQ ID NO:22, SEQ ID NO:23, SEQ ID NO:24, or SEQ ID NO:25.

A twenty-eighth embodiment is an expression system where the terminator sequence includes or consists of the nucleotide sequence set forth in SEQ ID NO:19.

A twenty-ninth embodiment is an expression system where the expression cassette includes SEQ ID NO:30, or a polynucleotide sequence having at least about 75%, 80%, 85%, 90%, 95%, 98%, or 99% sequence identity to SEQ ID NO:30.

A thirtieth embodiment is an expression system where the expression cassette consists of a polynucleotide sequence having 100% sequence identity to SEQ ID NO:30.

A thirty-first embodiment is an expression system, further including a viral vector.

A thirty-second embodiment is an expression system where the viral vector is an adenoviral vector, a retroviral vector, or a simian virus 40 (SV40) viral vector.

A thirty-third embodiment is an expression system where the host cell is a bacterial cell or a mammalian cell.

A thirty-fourth embodiment is an expression system where the host cell is a human cell.

A thirty-fifth embodiment is a pharmaceutical composition including the suppressor tRNA molecule, the polynucleotide sequence, or the expression system of any of the preceding embodiments, or a combination thereof.

A thirty-sixth embodiment is method of treating a genetic disease in a subject including administering to the subject the suppressor tRNA molecule, the polynucleotide sequence, or the expression system of any of the preceding embodiments, or a combination thereof, where the genetic disease is characterized by the presence of a premature termination codon in an mRNA template encoding a polypeptide, and where the premature termination codon replaces a sense codon in the mRNA template in the subject.

A thirty-seventh embodiment is a method where the disease is further characterized by the presence of a truncated polypeptide or the absence of the full-length polypeptide.

A thirty-eighth embodiment is a method where the premature termination codon includes the stop codon UAG, UAA, or UGA.

A thirty-ninth embodiment is a method where the suppressor tRNA molecule includes the nucleotide sequence: a) CUA, where three nucleotide residues base pair with the stop codon UAG, b) UUA, where the three nucleotide residues base pair with the stop codon UAA, or c) UCA, where the three nucleotide residues base pair with the stop codon UGA.

A fortieth embodiment is a method where the genetic disease is a cancer.

A forty-first embodiment is a method where the cancer is colorectal cancer, breast cancer, or ovarian cancer.

A forty-second embodiment is a method where the genetic disease is beta-thalassemia, cystic fibrosis, Duchenne muscular dystrophy, Fanconi anemia, hemophilia A, hemophilia B, Marfan syndrome, Menkes' disease, mucopolysaccharidosis type I-Hurler, neurofibromatosis type 1, osteogenesis imperfecta, retinitis pigmentosa, Rett syndrome, Usher syndrome, Wilson's disease, or any combination thereof.

A forty-third embodiment where the subject is human.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1. Schematic depicting incorporation of alanine (Ala) identity elements into the acceptor stem of wild-type pyrrolysine tRNA (tRNA^{Pyl}) by mutagenesis and charging of the resultant engineered tRNA^{Pyl} with alanine by alanyl-tRNA synthetase (AlaRS) derived from a human or E. coli.

FIG. 2. Schematic depicting mutagenesis of the Methanosarcina mazei tRNA^{Pyl} acceptor stem by introducing alanine identity elements, as indicated by arrows, in the production of first generation suppressor tRNA^{Pyl}-tRNA^{Ala} chimeras (V1.1, V1.2, and V1.3).

FIG. 3. A map of an exemplary plasmid, which includes a GFP reporter gene containing the UAG stop codon at position 2, which was used to measure premature termination codon (PTC) suppression by disclosed chimeric tRNAs in E. coli.

FIG. 4. Bar graph showing GFP expression in cells expressing either wild-type GFP (WT) in the absence of exogenous tRNA (NA) or GFP containing a PTC (UAG stop codon) at position 2 in the presence of a first-generation chimeric tRNA, V1.1, V1.2, or V1.3.

FIG. 5. Schematic depicting the structure of Methanomethylophilus alvus tRNA^{Pyl} and mutagenesis of acceptor stems to incorporate alanine identity elements in the production of two second-generation chimeric tRNAs (V2.1 and V2.2), as indicated by arrows.

FIG. 6. Bar graph showing GFP expression in cells expressing a wild-type GFP gene (WT) or a GFP gene with a PTC at position 2 (UAG) following treatment with a chimeric suppressor tRNA, V2.1 or V2.2.

FIG. 7. Bar graph showing percentage amino acid incorporation at PTC-encoded position 2 of the GFP gene, as determined by mass spectrometry.

FIG. 8. A map of an exemplary expression cassette containing three copies of a polynucleotide sequence encoding a disclosed suppressor tRNA and either a GFP reporter gene harboring a UAG PTC at position 150 or a SEAP reporter gene carrying a UAG PTC at position 40.

FIG. 9. A map of an exemplary plasmid containing a polynucleotide sequence encoding three copies of disclosed suppressor tRNA (PASS-tRNA), where the copies are arranged in tandem and each copy is operably linked to an h7SK promoter, a GFP gene containing a PTC mutation, and a viral vector.

FIG. 10. Fluorescence images of HEK 293 cells expressing a wild-type GFP gene tRNA (WT-GFP) or a GFP gene harboring a PTC at position 150 (UAG-GFP) and a previously reported engineered pyrrolysine tRNA molecule (tRNA^{Pyl}), a previously reported engineered tyrosine tRNA molecule (tRNA^{Tyr}), or an exemplary disclosed suppressor tRNA (tRNAV2.1).

FIG. 11. Bar graph showing relative GFP expression in HEK 293 cells treated with an engineered tRNA^{Pyl} molecule, an engineered tRNA^{Tyr} molecule, or exemplary disclosed suppressor tRNAs tRNAV2.1 or tRNAV2.2, where the cells express either WT-GFP or UAG-GFP.

FIG. 12. A map of an exemplary plasmid containing a polynucleotide sequence encoding three copies of disclosed chimeric tRNA (PASS-tRNA), where the copies are arranged in tandem and each copy is operably linked to an h7SK promoter, a SEAP gene containing a PTC mutation, and a viral vector.

FIG. 13. Bar graph showing relative SEAP expression in HEK 293 cells treated with WT-tRNA^{Pyl}, tRNA^{Tyr}, or exemplary disclosed suppressor tRNAs tRNAV2.1 or tRNAV2.2, where the cells express either a wild-type SEAP reporter (WT-SEAP) or a SEAP reporter with a UAG PTC at position 40 (UAG-SEAP).

FIG. 14. Bar graph showing relative SEAP expression in HEK 293 cells treated with WT-tRNA^{Pyl}, tRNAV2.1, or tRNA^{Ala}, a human alanine tRNA variant that is mutated to recognize the UAG stop codon, where the cells express either WT-SEAP or a SEAP reporter with a UAG PTC at position 40 (UAG-SEAP).

FIG. 15. Schematic depicting position 2 of GFP mutated to any of the three termination codons (UAG, UAA, and UGA).

FIG. 16. GFP expression in E. coli cells co-expressing either wild-type GFP (WT-GFP) or PTC-containing GFP (UAG-GFP, UAA-GFP, or UGA-GFP) and exemplary suppressor tRNA V2.1

FIG. 17. Schematic depicting anticodon mutations of an exemplary suppressor RNA,tRNAV2.1, to facilitate recognition of PTCs UAG, UAA, and UGA.

FIG. 18. Bar graph showing relative SEAP expression in HEK 293 cells treated with anticodon variants of previously reported tRNA^{Pyl} and tRNA^{Tyr} comparator molecules or exemplary disclosed chimeric tRNA^{Pyl} molecule tRNAV2.1, where the cells express either WT-SEAP or a SEAP reporter gene having a UAG, UAA, or UGA PTC at position 40 (UAG-SEAP, UAA-SEAP, and UGA-SEAP, respectively.

FIG. 19. Bar graph showing relative viability of HEK 293 cells expressing previously reported tRNA^{Pyl} and tRNA^{Tyr} comparator molecules or exemplary disclosed chimeric tRNA Pyl molecules tRNAV2.1 or tRNAV2.2, as determined by an MTT assay.

FIG. 20. (A) Image of protein separation gel showing p53 protein expression of full-length (FL) and truncated (Tr) p53 in cell transfected with PTC-containing TP53cDNA; (B) Bar graph depicting p53 transactivation activity in cells harboring TP53 cDNA with PTCs at the indicated positions. All data are shown for cells with (+) and without (−) co-expression of a sup-RNA.

FIG. 21. (A) Image of protein separation gel showing p53 protein expression for full-length (FL) and Cap Binding Complex 80 (CBP80) when Sup-tRNAs are present (+) or absent (−); (B) Bar graph depicting p53 transactivation activity in cells harboring homozygous mutations in endogenous TP53.

FIG. 22. (A) Bar graph depicting MDM2 and p21 mRNA expression levels in cells harboring PTC mutations in endogenous TP53 with (+) and without (−) co-expression of a sup-tRNA in C2bbel cells; (B) Bar graph depicting MDM2 and p21 mRNA expression levels in cells harboring PTC mutations in endogenous TP53 with (+) and without (−) co-expression of a sup-tRNA and Nutlin-3a (10 μM) treatment in SK-MES-1 cells; (C) Image of protein separation gel showing protein p21 and CBP80 protein expression in cells harboring PTC in endogenous TP53. (*p<0.05, **p<0.005, ns=not significant, unpaired t-test)

FIG. 23. Bar graph depicting relative fluorescence intensity of AAV-2 delivery of active sup-tRNAs to NCI-H1299 lung cancer cells.

FIG. 24. (A) Schematic representation of the plasmid system used to measure PTC suppression in BRCA1; (B) Bar graph depicting BRCA1 activity in HEK 293 cells expressing BRCA1 with a UAG PTC at the indicated position with (+) and without (−) co-expression of tRNA^{V2.1}. Data are displayed as the mean±SEM of three biological replicates. (*p<0.05, **p<0.005, ns=not significant, unpaired student's t-test)

FIG. 25. (A) Bar graph depicting ribosomae readthrough scores determined for HEK 293 cells transfected with an empty vector (−) or a vector harboring three copies of PASS-tRNA^{V2.1} (+). Data are pooled from two biological replicates; (B) Volcano plot depicting log 2 fold change in gene expression in HEK 293 cells treated with PASS-tRNA^{V2.1} and untreated cells. Data are from two biological replicates.

### DETAILED DESCRIPTION

Premature termination codons (PTCs) are stop codons that interrupt a protein-coding gene due to genetic mutation. These mutations cause protein synthesis to terminate prematurely, leading to an incomplete, non-functional protein product. Nearly 10-15% of genetic diseases, such as cystic fibrosis, Duchenne muscular dystrophy, and many cancers, are caused by PTCs. See, e.g., Martins-Dias & Romao, Cellular and Molecular Life Sciences, 2021. 78 (10): 4677-4701 and Porter & Leuck, Wiley Interdiscip Rev RNA, 2021. 12 (4): p. e1641. Given their role in diverse genetic disorders, multiple therapeutic strategies have been investigated to treat PTC-related diseases (Martins-Dias & Romao, Cellular and Molecular Life Sciences, 2021. 78 (10): 4677-4701). One therapeutic approach that has gained widespread attention recently is based on suppressor transfer RNAs. See, e.g., Dolgin, Nature Biotechnology, 2022. 40 (3): 283-286 and Cross, Chemical & Engineering News, 2021, 99 (34). Suppressor tRNA molecules can “readthrough” or “suppress” stop codons, preventing protein synthesis from terminating prematurely. See, e.g, Porter & Leuck, Wiley Interdiscip Rev RNA, 2021; Ko et al., Mol Ther Nucleic Acids, 2022. 28: p. 685-701; Sako et al., Nucleic Acids Symp Ser (Oxf), 2006 (50): p. 239-40; Shi et al., Nat Biomed Eng, 2022. 6 (2): 195-206; Lueck et al., Nat Commun, 2019. 10 (1): 822; Katrekar & Mali, Molecular Therapy, 2018. 26 (5): p. 86-86.

In one aspect, disclosed herein are chimeric suppressor tRNA molecules that restore protein synthesis from nucleotide sequences, such as DNA sequences and mRNA transcribed therefrom, containing PTCs. The disclosed exemplary chimeric suppressor tRNA molecules combine elements of the human alanine tRNA and an archaeal pyrrolysine tRNA, and are therefore also referred to as chimeric tRNA molecules. Pyrrolysine tRNA (RNA^{Pyl}) is a naturally occurring suppressor tRNA that translates the UAG termination codon with the amino acid pyrrolysine (Pyl). See, e.g., Tharp et al, RNA Biology, 2018. 15 (4-5): 441-452. In nature, tRNA^{Pyl} is charged with Pyl by an enzyme called pyrrolysyl-tRNA synthetase (PylRS). Pyl is found sparsely in bacteria and archaeal species, but it is absent in all eukaryotes, including humans. Engineered PylRS variants have been shown to recognize over 100 unnatural amino acids (uAAs) as substrates. See, e.g., Krahn et al., Enzymes, 2020. 48: p. 351-395 and Wan et al., Biochimica Et Biophysica Acta-Proteins and Proteomics, 2014. 1844 (6): 1059-1070. These engineered PylRS variants, together with tRNA^{Pyl}, can install uAAs into proteins by suppressing the UAG stop codon, as reviewed by Krahn et al., Enzymes, 2020. 48: p. 351-395.

Another exemplary use of the tRNAs disclosed herein is restoring function of tumor suppressor gene, p53. P53 is a regulatory protein that prevents cancer formation, but that is often mutated in human cancers. The mutations observed in p53 are frequently nonsense mutations giving rise to a stop codon rather than a codon specifying the native amino acid.

Several suppressor tRNAs have been engineered to suppress PTCs in humans, but while these previously reported suppressor tRNAs have been shown to revert disease phenotypes, they suffer from low suppression efficiency. One approach in the development of such suppressor tRNAs has been to repurpose tRNAs that do not naturally suppress stop codons. See, e.g., Albers et al., Nature Communications, 2021. 12 (1). For example, a recently reported suppressor tRNA was developed by altering the anticodon of the human tyrosine tRNA to recognize the UAG termination codon instead of the normal tyrosine codon (Wang et al., Nature, 2022. 604 (7905): 343). While this tRNA showed efficacy in mice, overall suppression of PTCs was low.

Described herein is a new class of suppressor tRNAs based on tRNA^{Pyl} with superior PTC suppression activity using tRNA molecules that have naturally evolved to suppress stop codons. The present work, e.g., as described in the Examples section, shows that the disclosed tRNA molecules can suppress different types of PTCs in human cells and display superior suppression efficiency of PTCs relative to a state-of-the-art suppressor tRNA. While tRNA_{Pyl} has been used for PTC suppression in human cells, this system has significant limitations that preclude its use as a therapeutic. See, e.g., Shi et al., Nat Biomed Eng, 2022. 6 (2): 195-206 and Katrekar & Mali, Molecular Therapy, 2018. 26 (5): p. 86-86. In one example, this system requires the delivery of both tRNA^{Pyl} and PylRS to diseased cells, and the large size of the PylRS protein complicates delivery. Additionally, this system requires simultaneously dosing diseased cells with a uAA. At the time of this disclosure, to the knowledge of the inventors, no uAAs have been approved for human use, as their biological safety has yet to be demonstrated. Among other aspects, the engineered tRNA disclosed herein, polynucleotides and vectors for expression of the same, and use of the disclosed suppressor tRNAs, overcome such limitations of previous technology.

Barriers that prevent tRNA^{Pyl} from being used as a therapeutic for treating diseases caused by PTCs include (1) the need for the simultaneous delivery of the PylRS enzyme, which aminoacylates tRNA^{Pyl}, and (2) the need for dosing diseased cells with an unnatural amino acid. Employing a unique solution for overcoming these barriers, tRNA^{Pyl} was converted into a substrate for a human aminoacyl-tRNA synthetase (aaRS), facilitating aminoacylation by an endogenous human aaRS with a natural amino acid, and thereby bypassing the requirements of PylRS delivery and uAA dosage. Accordingly, now described are tRNA^{Pyl} variants that can be aminoacylated by a human aaRS.

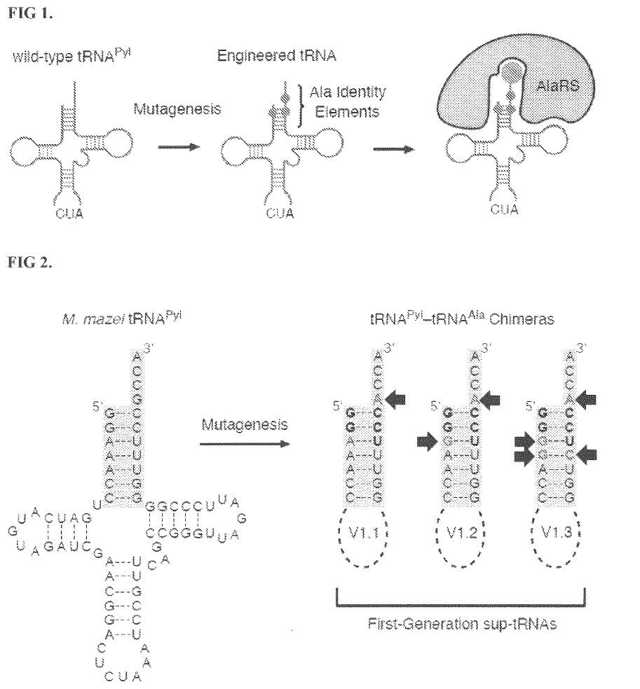

aaRSs recognize their substrate tRNAs using a subset of nucleotides within the tRNA known as identity elements. Identity elements are described, e.g., by Giege et al., Nucleic Acids Research, 1998. 26 (22): p. 5017-5035; Bonnefond et al., Biochimie. 2005 September-October; 87 (9-10): 873-83; and Martin et al., RNA. 1996 September; 2 (9): 919-27. Each aaRS recognizes a unique set of identity elements, and this specificity helps to ensure that an aaRS only interacts with its substrate tRNA and rejects numerous other tRNAs present in the cell. One strategy for converting tRNA^{Pyl} into a substrate for a human aaRS was to transplant identity elements from human tRNAs into tRNA Pyl to generate synthetic, chimeric tRNAs, as depicted in FIG. 1.

The disclosed chimeric molecules can be introduced to a cell in a variety of ways. In one example, disclosed chimeric molecules are introduced to a cell as RNA, such as by delivering the mature tRNA molecule to the cell. In another example, disclosed chimeric molecules are introduced to a cell as RNA, such as by expressing a DNA polynucleotide sequence in a host cell, where expression of the tRNA molecule is facilitated by an expression system.

In some aspects, it is advantageous for the disclosed chimeric suppressor tRNA molecules to be recognized by a defined human aaRS and aminoacylated by the same. Aminoacylation of a disclosed suppressor tRNA molecule by a human aaRS, such as charging a disclosed chimeric suppressor tRNA molecule with a desired amino acid can be determined according to methods known to one of skill in the art. For example, mass spectrometry may be used to confirm incorporation of a desired amino acid at the position encoded by a PTC. In other aspects, it is advantageous for the disclosed chimeric molecules to efficiently suppress PTCs, like wild-type tRNA^{Pyl}. Suppression of a PTC can be determined according to various methods available to one of skill in the art. For example, suppression of PTCs by disclosed chimeric tRNA molecules can be assessed with use of a reporter, such as a nonsense-suppression reporter, like a GFP or alkaline phosphatase gene modified to include a PTC. Alternatively, PTC-suppressing activity of disclosed chimeric RNA molecules may be evaluated by measuring relative production of a full-length protein, such as expression of a gene harboring a PTC mutation, following exposure to a suppressor tRNA molecule in comparison to baseline expression of the gene. See, e.g., Ko et al., Mol Ther Nucleic Acids. 2022 Jun. 14; 28:685-701.

Herein, the term “suppressor tRNA” refers to a tRNA that alters the translation of a messenger RNA (mRNA) in a protein synthesis process, e.g., by providing a mechanism for incorporating an amino acid into a polypeptide chain in response to a specific codon, such as a stop codon or premature termination codon.

Herein, the terms “suppress,” “suppressor,” “suppression,” and the like refer to reducing, eliminating, or silencing of the effects of a PTC mutation, e.g., by facilitating translation of an mRNA template containing a PTC mutation to a full-length, non-truncated protein, such as by recognition and readthrough of a PTC by a “suppressor” tRNA.

Herein, the terms “readthrough,” “read-through,” “reads through,” and the like refer to a process in which a tRNA molecule continues translating mRNA into a protein upon recognizing a PTC, such as upon base pairing between the tRNA molecule's anticodon loop and a complementary stop codon, by incorporating an amino acid at the site of the PTC. Rather than terminating translation prematurely, the tRNA molecule “reads through” the PTC in the mRNA template, thereby facilitating continued translation of mRNA to protein and suppressing the PTC mutation.

Herein, the terms “charges,” “charging,” and the like refer to aminoacylation of a tRNA molecule, such as the attachment of an amino acid to a tRNA molecule, an enzymatic process which is catalyzed by an aminoacyl-tRNA synthetase, also known as tRNA-ligase. Charging a tRNA molecule includes two steps. First, the tRNA synthetase binds to its cognate amino acid (AA) and a molecule of ATP, which catalyzes the condensation of aminoacyl adenylate (AA-AMP) and releases inorganic pyrophosphate (PPi). Then, the activated amino acid of AA-AMP is transferred to the cognate tRNA with the release of adenosine monophosphate (AMP). The product of this reaction is an aminoacyl-tRNA or an aminoacylated tRNA, which may then be delivered to a ribosome by elongation factors to take part in protein synthesis.

Herein, the term “chimeric,” such as with reference to the term “chimeric tRNA molecule,” refers to a molecule composed of components derived from at least two genetically distinct sources, such as a human source and a non-human source. In some embodiments, disclosed chimeric tRNA molecules comprise genetic elements from human (RNA and archaeal RNA.

Herein, the term “human (RNA identity element” refers to a specific molecular feature a human tRNA molecule, such as a structural motif, nucleotide residue, or sequence of nucleotide residues, which facilitates recognition and interaction of the (RNA molecule with a specific aminoacyl-tRNA synthetase enzyme. Generally, the identity of a tRNA is determined by a small number of nucleosides, and more precisely by chemical groups carried by these nucleosides that can interact with amino acids on tRNA synthetase enzymes. The concept of tRNA identity is discussed, e.g., by Giegé & Frugier, “Transfer RNA Structure and Identity.” In: Madame Curie Bioscience Database [Internet]. Austin (TX): Landes Bioscience; 2000-2013.

The terms “sequence identity” or “identical,” in the context of two or more nucleic acid or polypeptide sequences, refer to two or more sequences or subsequences that are the same or have a specified percentage of nucleotides or amino acid residues that are the same, such as when compared and aligned for maximum correspondence. Such correspondence can be measured according to sequence comparison algorithms and tools available to one of skill in the art, e.g., BLAST, or by visual inspection.

“Therapeutically effective amount” or “therapeutically effective dose” of a composition, e.g. a composition comprising a disclosed tRNA molecule or an expression system encoding a suppressor tRNA molecule for expression in a cell, refers to an amount that is effective to achieve a desired therapeutic result. The desired therapeutic result will vary according to the condition being treated. However, exemplary indications of therapeutic effectiveness include reduced production of truncated and/or non-functional proteins and increased production of full-length and/or functional proteins. Therapeutically effective amounts will typically depend upon the IC₅₀ and safety profile of the specific agent being administered.

Suppressor tRNA Molecules

In some aspects, provided herein are suppressor tRNA molecules, which include microbial tRNA molecules modified to include human identity elements, such as human tRNA identity elements. Herein, “tRNA molecule” and “tRNA molecules” may be referenced to simply as “tRNA or “tRNAs,” respectively. In some embodiments, a disclosed suppressor tRNA molecule may be introduced directly to a cell, such as in the form of a mature tRNA molecule, e.g., without expressing a polynucleotide sequence encoding the sup-tRNA in a host cell.

In some embodiments, disclosed suppressor tRNA molecules comprise a wild-type or mutant microbial tRNA which has been engineered to include human tRNA identity elements. In some embodiments, engineering the wild-type or mutant microbial tRNA molecule to include a human tRNA identity element comprises mutating the acceptor stem of the tRNA molecule. In some embodiments, engineering the wild-type or mutant microbial tRNA molecule to include a human tRNA identity element does not comprise mutating the anticodon loop of the tRNA molecule.

In some embodiments, disclosed suppressor tRNA molecules comprise a wild-type or mutant archaeal tRNA which has been engineered to include a human tRNA identity element. In some embodiments, the wild-type or mutant archaeal tRNA is naturally aminoacylated with a canonical amino acid. In some embodiments, the wild-type or mutant archaeal tRNA is naturally aminoacylated with a non-canonical amino acid. In some embodiments, disclosed suppressor tRNA molecules comprise a wild-type or mutant bacterial tRNA molecule which has been engineered to include a human tRNA identity element. In some embodiments, the wild-type or mutant bacterial tRNA molecule is naturally aminoacylated with a non-canonical amino acid.

In some embodiments, disclosed suppressor tRNA molecules comprise a wild-type or mutant bacterial tRNA that is naturally aminoacylated with a non-canonical amino acid, wherein the non-canonical amino acid is 4-azido-L-phenylalanine, p-acetylphenylalanine, p-azido-L-phenylalanine, p-propargyloxyphenylalanine, p-carboxymethyl-L-phenylalanine, O-methyl-L-tyrosine, pyrrolysine, selenocysteine, selenomethionine, pyrroline-5-carboxylate, seryl tRNA for amber codon (Ser-tRNA\hat{ }Amb), 4-methyl-L-proline.

In some embodiments, disclosed suppressor tRNA molecules comprise a wild-type or mutant archaeal RNA that is naturally aminoacylated with a non-canonical amino acid, wherein the non-canonical amino acid is pyrrolysine, selenocysteine, 4-methyl-L-proline, N-formylmethionine, 3-methylhistidine, norleucine, citrulline, ornithine, homocysteine, or a beta-amino acid, such as an amino acid having an amino group attached to the beta carbon instead of the alpha carbon.

In some embodiments, disclosed suppressor tRNA molecules comprise a wild-type or mutant pyrrolysyl tRNA molecule which has been modified to include a human tRNA identity element, such as a bacterial tRNA molecule or archaeal tRNA molecule, thereby producing a suppressor tRNA molecule. In some embodiments, modifying the wild-type or mutant pyrrolysyl RNA molecule to include a human tRNA identity element comprises mutating the acceptor stem of the tRNA molecule. In some embodiments, modifying the wild-type or mutant pyrrolysyl tRNA molecule to include a human tRNA identity element does not comprise mutating the anticodon loop of the tRNA molecule.

In some embodiments, the wild-type or mutant pyrrolysyl tRNA molecule, or a homolog thereof, is native to a strain of methanogenic archaea. In some embodiments, the mutant pyrrolysyl tRNA molecule is a modified version of the wild-type or homologous pyrrolysyl tRNA molecule derived from a strain of methanogenic archaea. In some embodiments, the strain of methanogenic archaea is a species of Candidatus Methanoplasma, Desulfitobacterium, Methanomethylophilus, Methanosarcina, or Methanosarcinaceae. In some embodiments, the strain of methanogenic archaea is Candidatus Methanoplasma termitum, Desulfitobacterium hafniense, Methanomethylophilus alvus, Methanosarcinaceae archaeon, Methanosarcina barkeri, or Methanosarcina mazeii. In some embodiments, the strain of methanogenic archaea is Methanomethylophilus alvus.

In some embodiments, the mutated acceptor stem comprises a region of unpaired nucleotides comprising four nucleotides on a single strand and a region of base-paired nucleotides comprising 14 paired nucleotides. In some embodiments, the region of unpaired nucleotides comprises at least one but less than four point mutations. In some embodiments, the region of unpaired nucleotides comprises a total of one, two, or three point mutation(s). In some embodiments, the region of unpaired nucleotides comprises a guanine to adenine point mutation. In some embodiments, the region of unpaired nucleotides has a total of one point mutation consisting of a guanine to adenine point mutation. In some embodiments, the region of unpaired nucleotides consists of four nucleotides comprising a total of one guanine to adenine point mutation.

In some embodiments, the region of base-paired nucleotides comprises at least one but less than seven point mutations. In some embodiments, the region of base-paired nucleotides comprises a total of one, two, three, four, five, or six point mutation(s). In some embodiments, the region of base-paired nucleotides comprises at least one but less than four uracil to cytosine point mutations or at least one but less than four cytosine to uracil point mutations. In some embodiments, the region of base-paired nucleotides comprises at least one but less than four uracil to cytosine point mutations and at least one but less than four cytosine to uracil point mutations. In some embodiments, the region of base-paired nucleotides consists of 14 paired nucleotides, such as seven base pairs of nucleotides, comprising one uracil to cytosine point mutation and one cytosine to uracil point mutation. In some embodiments, the region of base-paired nucleotides consists of 14 paired nucleotides comprising two uracil to cytosine point mutations and one cytosine to uracil point mutation.

In some embodiments, the region of unpaired nucleotides consists of four nucleotides comprising a total of one guanine to adenine point mutation; and the region of base-paired nucleotides consists of seven base pairs of nucleotides comprising one uracil to cytosine point mutation and one cytosine to uracil point mutation. In some embodiments, the region of unpaired nucleotides consists of four nucleotides comprising a total of one, such as a single, guanine to adenine point mutation; and the region of base-paired nucleotides consists of seven base pairs of nucleotides comprising two uracil to cytosine point mutations and one cytosine to uracil point mutation.

In some embodiments, a disclosed suppressor tRNA molecule comprises the mutations C66U/U67C/G69A. In some embodiments, a disclosed suppressor tRNA molecule further comprises the mutation U65C. In some embodiments, a disclosed suppressor tRNA molecule further comprises the mutation(s) C29U and/or U30C.

In some embodiments, a disclosed suppressor tRNA molecule comprises the mutations C66U/U67C/G69A, C29U/C66U/U67C/G69A, C29U/U30C/C66U/U67C/G69A, U65C/C66U/U67C/G69A, C29U/U65C/C66U/U67C/G69A, or C29U/U30C/U65C/C66U/U67C/G69A with reference to SEQ ID NO:1 or SEQ ID NO:8. In some embodiments, a disclosed suppressor tRNA molecule has mutations consisting of C66U/U67C/G69A, C29U/C66U/U67C/G69A, C29U/U30C/C66U/U67C/G69A, U65C/C66U/U67C/G69A, C29U/U65C/C66U/U67C/G69A, or C29U/U30C/U65C/C66U/U67C/G69A with reference to SEQ ID NO:1 or SEQ ID NO:8.

In some embodiments, disclosed suppressor tRNA molecules comprise an anticodon loop configured to base pair with a premature termination codon on an mRNA template encoding a polypeptide. In some embodiments, the anticodon loop comprises three nucleotide residues which are complementary to a termination codon, such as a premature termination codon. In some embodiments, the anticodon loop comprises the nucleotide sequence CUA, wherein the three nucleotide residues can base-pair with termination codon UAG. In some embodiments, the anticodon loop comprises the nucleotide sequence UUA, wherein the three nucleotide residues can base-pair with termination codon UAA. In some embodiments, the anticodon loop comprises the nucleotide sequence UCA, wherein the three nucleotide residues can base-pair with termination codon UGA.

In some embodiments, disclosed suppressor tRNA molecules comprise a mutant Methanomethylophilus alvus pyrrolysyl tRNA molecule comprising an anticodon loop configured to base pair with a premature termination codon on an mRNA template encoding a polypeptide.

In some embodiments, disclosed suppressor tRNA molecules are aminoacylated by a human aminoacyl-tRNA synthetase. In some embodiments, the suppressor tRNA molecule is charged, such as aminoacylated, with alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, disclosed suppressor tRNA molecules comprise a mutant Methanomethylophilus alvus pyrrolysyl tRNA molecule, wherein the mutant M. alvus pyrrolysyl tRNA molecule comprises a mutated acceptor stem comprising a human tRNA identity element. In some embodiments, the human tRNA identity element facilitates recognition of the human tRNA molecule by a human aminoacyl-tRNA synthetase.

In some embodiments, the human aminoacyl-tRNA synthetase is alanyl-tRNA synthetase, arginyl-tRNA synthetase, asparaginyl-tRNA synthetase, aspartyl-tRNA synthetase, cysteinyl-tRNA synthetase, glutaminyl-tRNA synthetase, glycyl-tRNA synthetase, histidyl-tRNA synthetase, isoleucyl-tRNA synthetase, leucyl-tRNA synthetase, lysyl-tRNA synthetase, methionyl-tRNA synthetase, phenylalanyl-tRNA synthetase, prolyl-tRNA synthetase, threonyl-tRNA synthetase, tryptophanyl-tRNA synthetase, tyrosyl-tRNA synthetase, or valyl-tRNA synthetase.

In some embodiments, the human aminoacyl-tRNA synthetase aminoacylates a disclosed suppressor tRNA molecule. In some embodiments, the disclosed suppressor tRNA molecule is aminoacylated with alanine, arginine, asparagine, aspartic acid, cysteine, glutamine, glutamic acid, glycine, histidine, isoleucine, leucine, lysine, methionine, phenylalanine, proline, threonine, tryptophan, tyrosine, or valine.

In some embodiments, the disclosed suppressor tRNA molecule translates a premature termination codon by incorporating the amino acid alanine, arginine, asparagine, aspartic acid, cysteine, glutamine, glutamic acid, glycine, histidine, isoleucine, leucine, lysine, methionine, phenylalanine, proline, threonine, tryptophan, tyrosine, or valine into the polypeptide at the site of the premature termination codon.

In preferred embodiments, the human aminoacyl-tRNA synthetase is human alanyl-tRNA synthetase, and the disclosed suppressor tRNA molecule is aminoacylated with alanine. In some embodiments, the disclosed suppressor tRNA molecule translates a premature termination codon by incorporating the amino acid alanine into the polypeptide at the site of the premature termination codon.

In one representative example of identity elements, alanine-tRNA, such as a tRNA molecule that is charged by human alanyl-tRNA synthetase with alanine, has include five nucleotides localized to the tRNA acceptor stem, which have been recognized as human alanine identity elements. See, e.g., Kumar et al., ACS Omega, 2019. 4 (13): p. 15539-15548 and Tamura et al., Mol Recognit, 1991. 4 (4): p. 129-32.

In some embodiments, a disclosed suppressor tRNA molecule comprises an RNA sequence having at least about 85%, 90%, 95%, 98%, or 99% sequence identity to SEQ ID NO:2, SEQ ID NO:3, SEQ ID NO:4, SEQ ID NO:5, SEQ ID NO:6, or SEQ ID NO:7. In some embodiments, a disclosed suppressor tRNA molecule comprises an RNA sequence comprising about 85%, 90%, 95%, 98%, or 99% sequence identity to SEQ ID NO:2, SEQ ID NO:3, SEQ ID NO: 4, SEQ ID NO:5, SEQ ID NO:6, or SEQ ID NO:7.

In some embodiments, a disclosed suppressor tRNA molecule comprises an RNA sequence having 100% sequence identity, such as identical, to SEQ ID NO:2, SEQ ID NO:3, SEQ ID NO:4, SEQ ID NO:5, SEQ ID NO:6, or SEQ ID NO:7. In some embodiments, a disclosed suppressor tRNA molecule consists of an RNA sequence having 100% sequence identity to SEQ ID NO:2, SEQ ID NO:3, SEQ ID NO:4, SEQ ID NO:5, SEQ ID NO:6, or SEQ ID NO: 7.

Herein, reference to SEQ ID NOs is meant to include reference to their full length sequence. For example, reference to a sequence having 100% sequence identity to SEQ ID NO:6 is to be understood as reference to a sequence having 100% sequence identity to the full length of SEQ ID NO:6. Sequences 1-7 omit the 3′ CCA tail of the tRNA molecule. The 3′ CCA tail is added enzymatically after transcription when the tRNAs are expressed in eukaryotes, such as by the enzyme tRNA nucleotidyltransferase, which is also referred to as the CCA enzyme. See, e.g., Hou, IUBMB Life. 2010; 62 (4): 251-260 and Kulandaisamy et al., Biochimie, 2023; 209:95-102. The tRNA genes for expression in bacteria included a sequence encoding the 3′ CCA.

In some embodiments, a disclosed suppressor tRNA molecule can be directly introduced to a cell as RNA. In some embodiments, the cell is a bacterial cell. In some embodiments, the bacterial cell is an E. coli cell. In some embodiments, the cell is a mammalian cell. In some embodiments, the mammalian cell is a human cell.

Exemplary methods of directly introducing a disclosed suppressor tRNA molecule to a cell include but are not limited to electroporation, microinjection, lipofection, transfection, nanoparticle delivery, antibody-conjugate delivery, cell-penetrating peptide delivery, vesicle delivery, and electrospray, including combinations thereof. See, e.g., Zhang & Yu, Curr Opin Biotechnol. 2008 October; 19 (5): 506-10, Kimble et al., Nature. 1982 Sep. 30; 299 (5882): 456-8, Wilson & Dutta, Front Mol Biosci. 2022; 9:888424, and Byun et al., Biochip J. 2022; 16 (2): 128-145.

### Recombinant Expression Systems

In some aspects, provided herein are polynucleotide sequences encoding suppressor tRNAs, such as disclosed suppressor tRNA molecules, including expression cassettes and plasmids comprising the same. In some embodiments, a disclosed expression system comprising a polynucleotide sequence encoding a suppressor tRNA molecule may be expressed in a cell, thereby introducing the suppressor tRNA to the cell. In some embodiments, the polynucleotide sequence encoding the suppressor tRNA molecule is stably integrated into the genome of a cell, such as a bacterial host cell or a mammalian host cell.

In some embodiments, a disclosed expression system comprises an expression cassette comprising a polynucleotide sequence encoding a suppressor tRNA molecule. In some embodiments, a disclosed expression cassette comprises one copy total of the nucleotide sequence encoding the suppressor tRNA molecule, i.e., the nucleotide sequence encoding the suppressor tRNA molecule is included only once and is not repeated. In some embodiments, a disclosed expression cassette comprises at least one copy of the nucleotide sequence encoding the suppressor tRNA molecule. In some embodiments, a disclosed expression cassette comprises at least two copies of the nucleotide sequence encoding the suppressor tRNA molecule. In some embodiments, a disclosed expression cassette comprises at least three copies of the nucleotide sequence encoding the suppressor tRNA molecule.

In some embodiments, a disclosed expression cassette comprises up to and including 10 total copies of the nucleotide sequence encoding the suppressor tRNA molecule. In some embodiments, a disclosed expression cassette comprises up to and including three, five, seven, or nine total copies of the nucleotide sequence encoding the suppressor tRNA molecule. In some embodiments, a disclosed expression cassette comprises one, two, three, four, five, six, or seven total copies of the nucleotide sequence encoding the suppressor tRNA molecule.

In some embodiments, a disclosed expression cassette comprises multiple copies of the polynucleotide sequence encoding the suppressor tRNA molecule, where the multiple copies are arranged in tandem. In some embodiments, the multiple copies are not arranged in tandem. In some embodiments, each of the multiple copies of the nucleotide sequence encoding the suppressor tRNA molecule is operably linked to a promoter. In some embodiments, the copies of polynucleotide sequence encoding the suppressor tRNA molecule are the same.

In some embodiments, a disclosed expression cassette comprises multiple polynucleotide sequences encoding different suppressor tRNA molecules. In some embodiments, the multiple polynucleotide sequences encoding different suppressor tRNA molecules are arranged in tandem. In some embodiments, the multiple polynucleotide sequences are not arranged in tandem. In some embodiments, each of the multiple polynucleotide sequences encoding different suppressor tRNA molecules is operably linked to a promoter.

In some embodiments, a polynucleotide sequence encoding the suppressor tRNA molecule comprises a polynucleotide sequence having at least about 85%, 90%, 95%, 98%, or 99% sequence identity to any one of SEQ ID NO:9, SEQ ID NO:10, SEQ ID NO:11, SEQ ID NO: 12, SEQ ID NO:13, and SEQ ID NO:14, or the complement thereof. In some embodiments, a polynucleotide sequence encoding the suppressor tRNA molecule comprises a polynucleotide sequence having about 85%, 90%, 95%, 98%, or 99% sequence identity to any one of SEQ ID NO: 9, SEQ ID NO:10, SEQ ID NO:11, SEQ ID NO:12, SEQ ID NO:13, and SEQ ID NO:14, or the complement thereof. In some embodiments, a polynucleotide sequence encoding the suppressor tRNA molecule comprises a sequence having 100% sequence identity to any one of SEQ ID NO: 9, SEQ ID NO:10, SEQ ID NO:11, SEQ ID NO:12, SEQ ID NO:13, and SEQ ID NO: 14, or the complement thereof. In some embodiments, a polynucleotide sequence encoding the suppressor tRNA molecule consists of a sequence having 100% sequence identity to any one of SEQ ID NO: 9, SEQ ID NO:10, SEQ ID NO:11, SEQ ID NO:12, SEQ ID NO:13, and SEQ ID NO: 14, or the complement thereof.

In some embodiments, a disclosed expression cassette comprises a nucleotide sequence encoding a suppressor tRNA molecule operably linked to a promoter. In some embodiments, the promoter recruits an RNA polymerase, such as RNA polymerase III. In some embodiments, the promoter is an RNA polymerase III type I, type II, or type III promoter.

In some embodiments, the promoter is an RNA polymerase III type III promoter. In some embodiments, the RNA polymerase III type III promoter is of mammalian origin. In some embodiments, the RNA polymerase III type III promoter is of human origin. In some embodiments, the RNA polymerase III type III promoter comprises a U6 promoter, an H1 promoter, a tRNA promoter, a 7SK promoter, a 7SL promoter, a Y3 promoter, a 5S rRNA promoter, an Ad2 VAI, a VAII promoter, or a derivative thereof. In some embodiments, the RNA polymerase III type III promoter comprises a human U6 promoter, a human H1 promoter, a human tRNA promoter, a human 7SK promoter, a human 7SL promoter, a human Y3 promoter, a human 5S rRNA promoter, a human Ad2 VAI, a human VAII promoter, or a derivative thereof.

RNA polymerase III type I, II, and III promoters recruit Pol III, with all three requiring the binding of TFIIIB, a 3-subunit TF containing the TATA box-binding protein (TBP). Type I and Type II promoters are gene-internal, while type III reside in the 5′ flanking region. See, e.g., Oler et al., Nat. Struct. Mol. Biol. 2010; 17:620-628 and Kulaberoglu et al., Front Genet. 2021 Jul. 6; 12:705122. These gene-external Pol III promoters use a defined +1 transcription start site and recognize T stretches as a termination signal. See, e.g., Schramm & Hernandez, Genes Dev. 2002 Oct. 15; 16 (20): 2593-620.

Nonlimiting examples of RNA polymerase III type III promoters include the U6 promoter, H1 promoter, tRNA promoter, 7SK promoter, 7SL promoter, Y3 promoter, 5S rRNA promoter, Ad2 VAI, and VAII promoter. Transcription elements necessary to transcribe human 7SK and H1 RNA genes have been studied by Murphy et al., Cell. 1987 Oct. 9; 51 (1): 81-7 and Baer et al., Nucleic Acids Res. 1990 Jan. 11; 18 (1): 97-103, respectively. Additionally, RNA polymerase III type III promoters are described, e.g., by Das, G et al., EMBO J. 1988 7:503-512; Hernandez, N., 1992, pp. 281-313, In S. L. McKnight and K. R. Yamamoto (ed.), Transcriptional regulation, vol. 1. Cold Spring Harbor Laboratory, Cold Spring Harbor, N.Y; Kunkel, Biochim. Biophys. Acta 1991 1088:1-9; Lobo & Hernandez, Cell 1989 58:55-67; Mattaj et al., 1988, Cell 55:435-442; Geiduschek & Kassavetis, 1992, pp. 247-280, In Transcriptional regulation. Monograph 22 (ed. S. L. McKnight and K. R. Yamamoto), Cold Spring Harbor Laboratory Press, Cold Spring Harbor, N.Y.).

In some embodiments, the promoter comprises the polynucleotide sequence represented in SEQ ID NO:15, or a polynucleotide sequence having at least about 80%, 85%, 90%, 95%, 98%, or 99% sequence identity to SEQ ID NO:15. In some embodiments, the promoter comprises the polynucleotide sequence represented in SEQ ID NO:15, or a polynucleotide sequence having about 80%, 85%, 90%, 95%, 98%, or 99% sequence identity to SEQ ID NO:15. In some embodiments, the promoter consists of a polynucleotide sequence having 100% sequence identity to SEQ ID NO:15.

In some embodiments, a disclosed expression cassette comprises a polynucleotide sequence encoding a suppressor tRNA molecule comprising the sequence represented in any one of SEQ ID NO: 9, SEQ ID NO:10, SEQ ID NO:11, SEQ ID NO:12, SEQ ID NO:13, and SEQ ID NO: 14, or the complement thereof, or a polynucleotide sequence having at least about 85%, 90%, 95%, 98%, or 99% sequence identity to any one of SEQ ID NO: 9, SEQ ID NO:10, SEQ ID NO: 11, SEQ ID NO:12, SEQ ID NO:13, and SEQ ID NO:14, or the complement thereof, operably linked to a promoter comprising the polynucleotide sequence represented in SEQ ID NO: 15, SEQ ID NO:16, or SEQ ID NO:17, or a polynucleotide sequence having at least about 80%, 85%, 90%, 95%, 98%, or 99% sequence identity to SEQ ID NO:15, SEQ ID NO:16, or SEQ ID NO:17. In some embodiments, a disclosed expression cassette comprises a polynucleotide sequence encoding a suppressor tRNA molecule comprising the sequence represented in any one of SEQ ID NO: 9, SEQ ID NO:10, SEQ ID NO:11, SEQ ID NO:12, SEQ ID NO:13, and SEQ ID NO:14, or the complement thereof, or a polynucleotide sequence having about 85%, 90%, 95%, 98%, 99%, or 100% sequence identity to any one of SEQ ID NO:9, SEQ ID NO:10, SEQ ID NO:11, SEQ ID NO:12, SEQ ID NO:13, and SEQ ID NO:14, or the complement thereof, operably linked to a promoter comprising the polynucleotide sequence represented in SEQ ID NO:15, SEQ ID NO:16, or SEQ ID NO:17, or a polynucleotide sequence having about 80%, 85%, 90%, 95%, 98%, 99%, or 100% sequence identity to SEQ ID NO:15, SEQ ID NO:16, or SEQ ID NO:17.

In some embodiments, a disclosed expression cassette further comprises a terminator sequence. Herein, the term “terminator sequence” may be used interchangeably with a “termination sequence.” In some embodiments, the terminator sequence flanks the 3′ end of the polynucleotide sequence encoding the suppressor tRNA molecule. In some embodiments, the terminator sequence is a poly-T termination signal. In some embodiments, the poly-T termination signal comprises at least 5, 7, 9, 11, 13, or 15 thymine residues. In some embodiments, the poly-T termination signal comprises up to and including 6, 8, 10, 12, 14, or 15 thymine residues. In some embodiments, the terminator sequence comprises the nucleotide sequence represented in any one of SEQ ID NOs: 18, 19, 20, 21, 22, 23, 24, or 25. In some embodiments, the terminator sequence consists of the nucleotide sequence represented in any one of SEQ ID NOs: 18, 19, 20, 21, 22, 23, 24, or 25.

In some embodiments, a disclosed expression cassette comprises SEQ ID NO:30 or the complement thereof, or a polynucleotide sequence having at least about 75%, 80%, 85%, 90%, 95%, 98%, or 99% sequence identity to SEQ ID NO:30 or the complement thereof. In some embodiments, a disclosed expression cassette comprises SEQ ID NO:30 or the complement thereof, or a polynucleotide sequence having about 75%, 80%, 85%, 90%, 95%, 98%, or 99% sequence identity to SEQ ID NO:30 or the complement thereof. In some embodiments, a disclosed expression cassette consists of a polynucleotide sequence having 100% sequence identity to SEQ ID NO: 30 or the complement thereof.

In some examples, disclosed DNA polynucleotides encoding suppressor tRNA molecules can be delivered to a cell by poration methods, e.g., electroporation, transfection, peptide-DNA complex delivery, e.g., cell-penetrating peptide delivery, lipid-mediated delivery, nanoparticle delivery, and delivery by injection, e.g., microinjection.

In other examples, a disclosed expression system includes a vector, such as an expression vector. Nonlimiting examples of the expression vector for inclusion in a disclosed expression system include but are not limited to eukaryotic vectors, prokaryotic vectors, e.g., bacterial vectors, and viral vectors, e.g., including but not limited to retroviral vectors, adenoviral vectors, adeno-associated viral vectors, lentivirus vectors (human and other including porcine), Herpes virus vectors, Epstein-Barr virus vectors, SV40 virus vectors, pox virus vectors, pseudotype virus vectors. Such vectors are further described, e.g., in U.S. Pat. No. 6,309,830B1.

In some embodiments, disclosed expression systems comprise a viral vector. In some embodiments, the viral vector is a DNA viral vector. In some embodiments, the viral vector is an RNA viral vector. In some embodiments, the viral vector is a retroviral vector. In some embodiments, the retroviral vector is derived from Moloney Murine Leukemia Virus, spleen necrosis virus, and Rous Sarcoma Virus, Harvey Sarcoma Virus, avian leukosis virus, human immunodeficiency virus, myeloproliferative sarcoma virus, and mammary tumor virus.

In some embodiments, disclosed expression systems comprise a simian virus 40 (SV40) viral vector. In some embodiments, disclosed expression systems comprise an SV40 promoter. In some embodiments, disclosed expression systems comprise an SV40 terminator sequence, such as an SV40 poly(A) signal.

In some embodiments, a disclosed polynucleotide encoding a suppressor tRNA as described herein and/or an expression cassette comprising the same are integrated, such as stably integrated, into the genome of a host cell. Stable integration refers to sustaining long-term expression of a transgene by integrating foreign DNA into the host nuclear genome. Accordingly, foreign DNA, or the transgene, is integrated into the genome, replicated alongside genomic DNA, and passed down to progeny. In one representative example relating to recombinant SV40-derived gene therapy vectors, durable transgene expression and vector integration is described by Strayer et al., Mol Ther. 2002 August; 6 (2): 227-37. In contrast, nucleic acids are not integrated into the host cell genome in transient transfection. In transient systems, foreign DNA is unable to replicate independently from the host's DNA and may persist only for a few days. See, e.g., Fus-Kajawa, Front Bioeng Biotechnol. 2021 Jul. 20; 9:701031.

### Compositions

In some aspects, provided herein are compositions, such as pharmaceutical compositions comprising disclosed suppressor tRNA molecules, expression systems, or a combination thereof. In some embodiments, a tRNA molecule or expression system is combined with a pharmaceutically acceptable carrier. The term “pharmaceutically acceptable” as used herein refers to those compounds, materials, compositions, and dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein refers to buffers, carriers, and excipients for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions, e.g., emulsions of oil and water, and various types of wetting agents. In some examples, the compositions may also include stabilizers and preservatives. Exemplary carriers, stabilizers, and adjuvants are described, e.g., in Martin, Remington's Pharmaceutical Sciences, 15^{th} Ed., Mack Publ. Co., Easton, Pa. 1975. Pharmaceutically acceptable carriers include buffers, solvents, dispersion media, coatings, isotonic and absorption delaying agents, and the like, that are compatible with pharmaceutical administration.

In some embodiments, a disclosed composition may contain excipients for modifying, maintaining or preserving, for example, the pH, osmolarity, viscosity, clarity, color, isotonicity, odor, sterility, stability, rate of dissolution or release, adsorption or penetration of the composition. In such embodiments, formulation materials include, but are not limited to, amino acids, e.g., glycine, glutamine, asparagine, arginine or lysine, antimicrobials, antioxidants, e.g., ascorbic acid, sodium sulfite or sodium hydrogen-sulfite, buffers, e.g., borate, bicarbonate, Tris-HCl, citrates, phosphates or other organic acids, bulking agents, e.g., mannitol or glycine, chelating agents, e.g., ethylenediamine tetraacetic acid (EDTA), complexing agents, e.g., caffeine, polyvinylpyrrolidone, beta-cyclodextrin or hydroxypropyl-beta-cyclodextrin), fillers, monosaccharides, disaccharides, and other carbohydrates, e.g, glucose, mannose or dextrins, proteins, e.g., serum albumin, gelatin or immunoglobulins, coloring, flavoring, and diluting agents, emulsifying agents, hydrophilic polymers, e.g., polyvinylpyrrolidone, low molecular weight polypeptides, salt-forming counterions, e.g., sodium, preservatives, e.g., benzalkonium chloride, benzoic acid, salicylic acid, thimerosal, phenethyl alcohol, methylparaben, propylparaben, chlorhexidine, sorbic acid or hydrogen peroxide, solvents, e.g., glycerin, propylene glycol or polyethylene glycol, sugar alcohols, e.g., mannitol or sorbitol, suspending agents, surfactants or wetting agents, e.g., pluronics, PEG, sorbitan esters, polysorbates such as polysorbate 20, polysorbate, triton, tromethamine, lecithin, cholesterol, tyloxapal, stability enhancing agents, e.g., sucrose or sorbitol, tonicity enhancing agents, e.g., alkali metal halides, preferably sodium or potassium chloride, mannitol sorbitol, delivery vehicles, diluents, excipients, and pharmaceutical adjuvants. See, e.g., Remington's Pharmaceutical Sciences, 18^{th} ed. Mack Publishing Company, 1990.

In some embodiments, a disclosed composition may contain nanoparticles, e.g., polymeric nanoparticles, liposomes, or micelles. See, e.g., Anselmo et al. Bioeng. Transl. Med. 2016; 1:10-29. In other embodiments, the composition does not comprise a nanoparticle or an aminolipid delivery compound, e.g., as described in U.S. Patent Publication No. 2017/0354672.

In some embodiments, the tRNA or expression vector introduced into the cell or administered to the subject is not conjugated to or associated with another moiety, e.g., a carrier particle, e.g., an aminolipid particle. In other embodiments, the tRNA or expression vector introduced into the cell or administered to the subject is not conjugated to or associated with another moiety, e.g., a carrier particle, e.g., an aminolipid particle. As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically, the moieties are attached either by one or more covalent bonds or by a mechanism that involves specific binding. Alternatively, a sufficient number of weaker interactions can provide sufficient stability for moieties to remain physically associated.

In some embodiments, a pharmaceutical composition may contain a sustained- or controlled-delivery formulation. Techniques for formulating sustained- or controlled-delivery means, such as liposome carriers, bio-erodible microparticles or porous beads and depot injections, are known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D (−)-3-hydroxybutyric acid. Sustained release compositions may also include liposomes that can be prepared by any of several methods known in the art.

In some examples, a method of delivering a nucleic acid molecule may be adapted for use with a tRNA. See e.g., Akhtar & Juliano, Trends Cell. Biol. 1992; 2 (5): 139-144 and PCT Publication No. WO94/02595. For example, the tRNA molecule can be modified or alternatively delivered using a drug delivery system to prevent the rapid degradation of the tRNA by endo- and exo-nucleases in vivo. tRNA molecules can be modified by chemical conjugation to lipophilic groups such as cholesterol to enhance cellular uptake and prevent degradation. tRNA molecules can also be conjugated to or otherwise associated with an aptamer. A tRNA can also be delivered using drug delivery systems, such as a nanoparticle, a dendrimer, a polymer, liposomes, or a cationic delivery system. Positively charged cationic delivery systems may facilitate binding of a tRNA molecule, which is negatively charged, and also enhance interactions at the negatively charged cell membrane to permit efficient uptake of a tRNA by the cell. Cationic lipids, dendrimers, or polymers can either be bound to the RNA, e.g., tRNA, or induced to form a vesicle or micelle (see e.g., Kim et al. Journal of Controlled Release 2008; 129 (2): 107-116) that encases the RNA. Methods of preparing and administering cationic-RNA complexes are described, e.g., by Sorensen et al. J. Mol Biol 2003; 327:761-766; Verma et al., Clin. Cancer Res. 2003; 9:1291-1300; Arnold et al. J. Hypertens. 2007; 25:197-205. Some non-limiting examples of drug delivery systems for systemic delivery of RNA, e.g., tRNAs including DOTAP (Sorensen et al. (2003) supra; Verma et al. (2003), supra), Oligofectamine, solid nucleic acid lipid particles (Zimmermann et al., Nature 2006; 441:111-114), cardiolipin (Chien et al., Cancer Gene Ther. 2005; 12:321-328; Pal et al., Int J. Oncol. 2005; 26:1087-1091), polyethyleneimine (Bonnet et al. Pharm. Res. 2008; 25 (12): 2972-82; Aigner, J. Biomed. Biotechol. 2006; 71659), Arg-Gly-Asp (RGD) peptides (Liu, Mol Pharm 2006; 3:472-487), and polyamidoamines (Tomalia et al., Biochem. Soc. Trans. 2007; 35:61-67; Yoo et al., Pharm. Res. 1999; 16:1799-1804). In some embodiments, a tRNA forms a complex with cyclodextrin for systemic administration. Methods for administration and pharmaceutical compositions of RNAs and cyclodextrins can be found in U.S. Pat. No. 7,427,605.

In some embodiments, disclosed compositions are provided in a dosage unit form. Disclosed compositions are formulated to be compatible with the intended route of administration. Exemplary of routes of administration include intravenous (IV), intradermal, inhalation, transdermal, topical, transmucosal, intrathecal and rectal administration. In some embodiments, a tRNA and/or expression vector is administered by injection. Formulations may be prepared by methods known in the pharmaceutical art. For example, see Remington's Pharmaceutical Sciences, 18th ed. (Mack Publishing Company, 1990). Formulation components suitable for parenteral administration include a sterile diluent, e.g., water for injection, saline solution, fixed oils, polyethylene glycols, glycerin, propylene glycol or other synthetic solvents; antibacterial agents, e.g., benzyl alcohol or methyl parabens; antioxidants, e.g., ascorbic acid or sodium bisulfite; chelating agents such as EDTA; buffers such as acetates, citrates or phosphates; and agents for the adjustment of tonicity, e.g., sodium chloride or dextrose.

For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol, e.g., for example, glycerol, propylene glycol, and liquid polyethylene glycol, and suitable mixtures thereof.

Disclosed compositions can be sterile, such as for local or systemic administration. In some embodiments, disclosed compositions are administered to a subject parenterally. Preparations for parenteral administration include sterile aqueous or non-aqueous solutions, suspensions, and emulsions. In some embodiments, disclosed compositions are administered to a subject subcutaneously or intravenously. Sterilization can be accomplished by any suitable method, e.g., filtration through sterile filtration membranes. Where the composition is lyophilized, filter sterilization can be conducted prior to or following lyophilization and reconstitution.

### Method of Use

In some aspects, provided herein are methods of using disclosed suppressor tRNA molecules, disclosed expression systems encoding suppressor tRNA molecules, disclosed compositions, and combinations thereof, such as in a method of treating a disease or disorder in a subject. In some embodiments, disclosed methods comprise administering a disclosed composition comprising a suppressor tRNA molecule and/or an expression system encoding the same to a subject in need thereof, such as a subject having a disease or disorder. In some embodiments, the disease or disorder is characterized by the presence of a point mutation. In some embodiments, the disease or disorder is characterized by a G to A point mutation which results in a premature termination codon. In some embodiments, the disease or disorder is characterized by a T to C point mutation which results in a premature termination codon.

In some embodiments, the disease or disorder is a genetic disease. In some embodiments, the disease is characterized by the presence of the premature termination codon in the mRNA template. In some embodiments, the premature termination codon replaces a sense codon in the mRNA template in the subject, thereby inhibiting protein production. In some embodiments, the disease or disorder is associated with production of at least one truncated protein, production of at least one non-functional protein, deficiency of at least one protein, such as protein deficiency, or a combination thereof.

In some embodiments, the disease or disorder is a cancer. In some embodiments, the cancer is colorectal cancer, breast cancer, ovarian cancer, or a combination thereof. In some embodiments, the cancer is hereditary. In some embodiments, the cancer is mediated at least in part by hereditary cancer syndrome. In some embodiments, the cancer is familial breast-ovarian cancer-1. In some embodiments, the cancer is familial breast-ovarian cancer-2.

In some embodiments, the disease or disorder is any of adenomatous polyposis, Alport syndrome, amyotrophic lateral sclerosis, Angelman syndrome, anemia, e.g., mediated by GFPD deficiency, AVPR2 nephrogenic diabetes insipidus, X-linked, beta-thalassemia, e.g., mediated by HBB deficiency, cardiac arrhythmia, Charcot-Marie-Tooth disease, congenital muscular dystrophy, homocystinuria, e.g., mediated by CBS deficiency, cystic fibrosis, dihydropyrimidine dehydrogenase deficiency, dilated cardiomyopathy, Duchenne muscular dystrophy, Ehlers-Danlos syndrome, epilepsy, episodic pain syndrome, Fanconi anemia, Gaucher disease, hereditary factor VIII deficiency disease, hereditary factor IX deficiency disease, hereditary fructosuria hemophilia A, hemophilia B, hypertrophic cardiomyopathy, e.g., primary familial hypertrophic cardiomyopathy, X-linked severe combined immunodeficiency, familial hypercholesterolemia, indifference to pain, congenital, autosomal recessive, insulin-dependent diabetes mellitus secretory diarrhea syndrome Joubert syndrome, juvenile polyposis syndrome, Kugelberg-Welander disease, Leigh disease, leukodystrophy, limb-girdle muscular dystrophy, Lynch syndrome, macular dystrophy, Marfan syndrome, Menkes' disease, mental retardation, e.g., X-linked, mucopolysaccharidosis type I-Hurler, Navajo neurohepatopathy, neurofibromatosis, e.g., neurofibromatosis type 1, Niemann-Pick disease, Noonan syndrome, osteogenesis imperfecta, Parkinson disease, e.g., Parkinson disease type II, primary ciliary dyskinesia, pulmonary hypertension, renal dysplasia, retinitis pigmentosa, Rett syndrome, spinal muscular atrophy, Tangier disease, tuberous sclerosis, UDP glucose-hexose-1-phosphate uridylyltransferase deficiency, Usher syndrome, Wilson's disease, Point mutations associated with the preceding diseases or disorders, which in no way should be perceived as limiting are available, e.g., in US20210163948A1, which is incorporated by reference.

Therapeutic effectiveness of a disclosed method of treatment may be evident to one of skill in the art and is variable dependent on the disease or disorder being treated. For example, subject criteria and therapeutic endpoints may be referenced from clinical literature or a clinical database, such as ClinicalTrials.gov. In one example, therapeutic efficacy may be evaluated by a reduction in the production truncated and/or non-functional proteins, an increase in the production of functional and/or full-length proteins, or a combination thereof, which may be detected according to methods known to one of skill in the art.

In some embodiments, disclosed methods comprise administering a composition comprising a therapeutically effective amount of a disclosed suppressor tRNA molecule to a subject in need thereof. In some embodiments, disclosed methods comprise administering a composition comprising a therapeutically effective amount of a disclosed suppressor tRNA molecule, such as a tRNA molecule comprising or consisting of SEQ ID NO:2, SEQ ID NO:3, SEQ ID NO:4, SEQ ID NO:5, SEQ ID NO:6, or SEQ ID NO:7, or an RNA sequence having at least about 85%, 90%, 95%, 98%, or 99% sequence identity to SEQ ID NO:2, SEQ ID NO:3, SEQ ID NO:4, SEQ ID NO:5, SEQ ID NO:6, or SEQ ID NO:7 to a subject in need thereof.

In some embodiments, disclosed methods comprise administering a composition comprising a therapeutically effective amount of a disclosed expression system to a subject in need thereof, such as an expression system comprising polynucleotides encoding a suppressor tRNA molecule for expression in a cell. In some embodiments, disclosed methods comprise administering a composition comprising a therapeutically effective amount of a disclosed expression system, such as a polynucleotide comprising or consisting of any one of SEQ ID NO: 9, SEQ ID NO:10, SEQ ID NO:11, SEQ ID NO:12, SEQ ID NO:13, SEQ ID NO:14, and SEQ ID NO: 30, or the complement thereof, or a polynucleotide sequence having at least about 75%, 80%, 85%, 90%, 95%, 98%, or 99% sequence identity to any one of SEQ ID NO: 9, SEQ ID NO: 10, SEQ ID NO:11, SEQ ID NO: 12, SEQ ID NO:13, SEQ ID NO:14, and SEQ ID NO:30, or the complement thereof.

In some embodiments, disclosed methods comprise administering a composition comprising a combination of a therapeutically effective amount of a disclosed suppressor tRNA molecule and a disclosed expression system comprising polynucleotides encoding at least one suppressor tRNA molecule to a subject in need thereof.

### Kits

In some aspects, provided herein a kits for performing any of the disclosed methods, in addition to instructions for carrying out the methods of the present disclosure and/or administering the vectors, recombinant expression systems, and compositions disclosed herein.

In some embodiments, a disclosed kit comprises agents necessary for the preservation of those components comprised therein, e.g., a buffering agent or a preservative. In some embodiments, each component of the kit is enclosed within an individual container. In some embodiments, the plurality of individual containers are packaged within a single package, along with instructions for interpreting the results of the assays performed using the kit. In some embodiments, the kits of the present disclosure may contain a written product on or in the kit container.

### EXAMPLES

### Example 1: Design of Suppressor tRNAs and Premature Termination Codon Readthrough Activity Thereof

Engineered suppressor tRNAs (sup-tRNA) can facilitate continued translation of an amino acid from an mRNA transcript where a stop codon, such as a premature termination codon, would typically be recognized. In contrast to previous approaches, which involve modification of tRNAs that do not naturally suppress stop codons, experimental sup-tRNAs were created by introducing alanine-tRNA identity elements into tRNA charged with pyrrolysine (tRNA^{Pyl}).

It was hypothesized that such a chimeric tRNA would (1) be aminoacylated by the human alanyl-tRNA synthetase (AlaRS) and (2) efficiently suppress UAG stop codons with the amino acid alanine, as depicted by FIG. 1. Alanine-tRNA identity elements include five nucleotides which are localized to the tRNA acceptor stem. See, e.g., Kumar et al., ACS Omega, 2019. 4 (13): p. 15539-15548 and Tamura et al., Mol Recognit, 1991. 4 (4): p. 129-32. Accordingly, introducing mutations to convert a tRNA^{Pyl} molecule into a substrate for AlaRS should not disrupt the tRNA molecule's anticodon. Additionally, the side chain of the amino acid alanine consists of only a methyl (CH₃) group, and it was hypothesized that the size of the side chain could minimize disruptions to the structure and function of the protein encoded by the PTC-containing gene. Furthermore, the alanine-tRNA identity elements are conserved in bacteria and eukaryotes, which could allow for aminoacylation of the engineered tRNAs by, e.g., Escherichia coli AlaRS and human AlaRS.

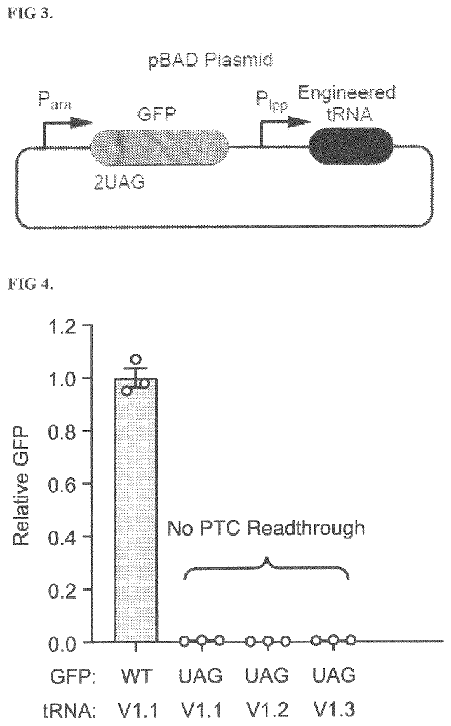

First-Generation sup-tRNAs: A series of chimeric sup-tRNAs was generated by transplanting different combinations of the human alanine identity elements into the tRNA^{Pyl} from M. mazei. Previously, tRNA^{Pyl} homologs from the archaeal species Methanosarcina mazei and Methanosarcina barkeri have been shown to suppress PTCs (Shi et al., Nat Biomed Eng, 2022. 6 (2): p. 195-206 and Katrekar & Mali, Molecular Therapy, 2018. 26 (5): p. 86-86. FIG. 2 shows the acceptor stem of three engineered tRNAs (V1.1, V1.2, and V1.3), with arrows indicating the point mutations. It was hypothesized that these mutations would enable aminoacylation of the chimeric tRNA^{Pyl} variants by AlaRS. A fluorescence-based assay was used to test whether the chimeric M. mazei sup-tRNAs could suppress a PTC mutation in the model gene green fluorescent protein (GFP). Specifically, the GFP mutant contained an in-frame UAG stop codon, as depicted in FIG. 3. In this assay, GFP should only be produced if the engineered sup-tRNAs readthrough the PTC, thereby suppressing the mutation. Alanine identity elements are identical in humans and E. coli. Accordingly, the PTC-suppressing activity of chimeric M. mazei tRNAs was assessed in E. coli. Surprisingly, the activity screen showed that none of the first-generation chimeric M. mazei sup-tRNAs were capable of suppressing or reading through the UAG PTC in the GFP gene, as shown in FIG. 4.

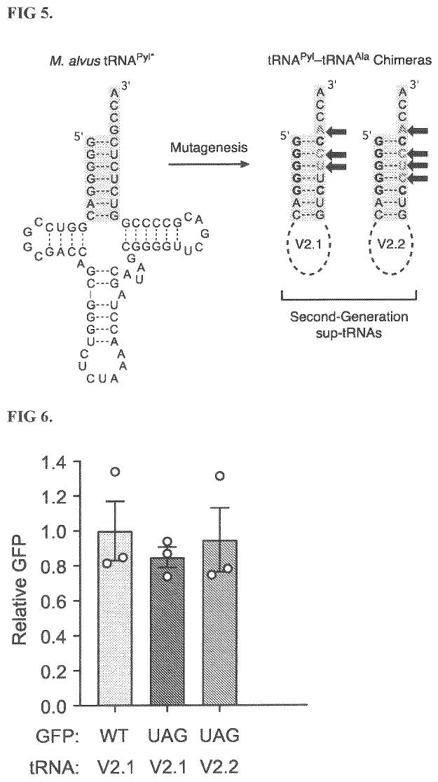

Second-Generation sup-tRNAs: A tRNA^{Pyl} homolog from a distantly related species known as Methanomethylophilus alvus was used in the design of a chimeric sup-tRNA molecule. M. alvus tRNA^{Pyl} has several unique structural features, including the lack of a base at the intersection of the acceptor and D arms, shortened D and T loops, and a bulge in the anticodon stem. See, e.g., Tharp et al., RNA Biol, 15, 441-452 and Borrel et al., Archaea, 2014, 374146. The structure of an engineered M. alvus tRNA^{Pyl} molecule, which was previously described by Willis & Chin, Nature Chemistry, 2018; 10 (8): 831-837 from is shown in FIG. 5.

Two second-generation chimeric sup-tRNAs (V2.1 and V2.2) were produced by inserting alanine identity elements into the engineered M. alvus tRNA^{Pyl} molecule, as depicted in FIG. 5. One such expression cassette containing the sup-tRNA V2.1 is set out in SEQ ID NO:30. These sup-tRNAs were evaluated in E. coli using the above-described GFP-based assay. To the knowledge of the inventors, this is the first study using M. alvus tRNA^{Pyl} to suppress PTCs for therapeutic applications.

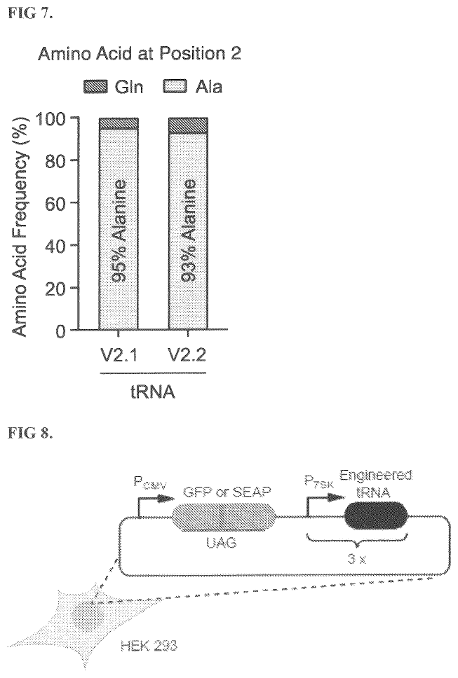

FIG. 6 shows suppression of the UAG PTC in the GFP gene by both chimeric M. alvus sup-tRNAs. Notably, these tRNAs showed exceptionally high efficiency in suppressing the PTC, restoring GFP expression to wild-type levels (GFP without a PTC mutation). Importantly, as shown in FIG. 7, mass spectrometry analysis of GFP expressed in these cells confirmed that alanine was the primary amino acid inserted at the position encoded by UAG. The presence of glutamine at the PTC-encoded position is likely a result of misincorporation at UAG codons due to near-cognate suppression in E. coli by endogenous tRNA^{Gln}, as observed, e.g., by Aerni et al., Nucleic Acids Res. 2015 January; 43 (2): e8 and Seki et al., bioRxiv, 2022.2010.2004.510903. Together, these results indicate that the engineered sup-tRNAs are recognized by the E. coli AlaRS and that the sup-tRNAs effectively suppress the PTC mutation by promoting continued translation upon recognition of the stop codon.

### Example 2: Suppression of the UAG Premature Termination Codon by Chimeric Suppressor M. Alvus tRNA^{Pyl} Molecules in Human Cells

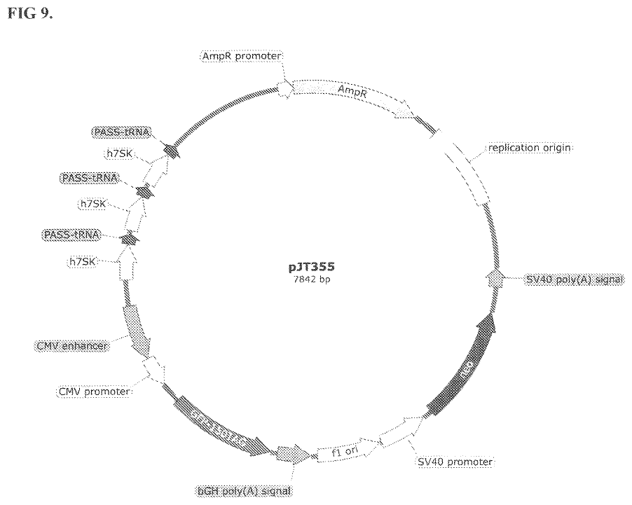

PTC Suppression in GFP: HEK 293 cells were used to determine whether the second-generation sup-tRNAs, i.e., the chimeric M. alvus tRNA^{Pyl} molecules described in Example 1, could suppress PTCs in human cells. FIGS. 8 and 9 show an expression cassette and a plasmid, respectively, engineered to include a sup-tRNA gene (M. alvus tRNAV2.1 or tRNAV2.2) and a GFP reporter gene containing a UAG PTC at position 150. HEK 293 cells were transiently transfected with the plasmid, and the resultant GFP expression was measured. Under these conditions, GFP expression should only occur if the sup-tRNAs can suppress the PTC in the GFP gene. (SEQ ID NOs: 26 and 27 show the CMV promoter and CMV enhancer for the expression cassette of FIG. 9)

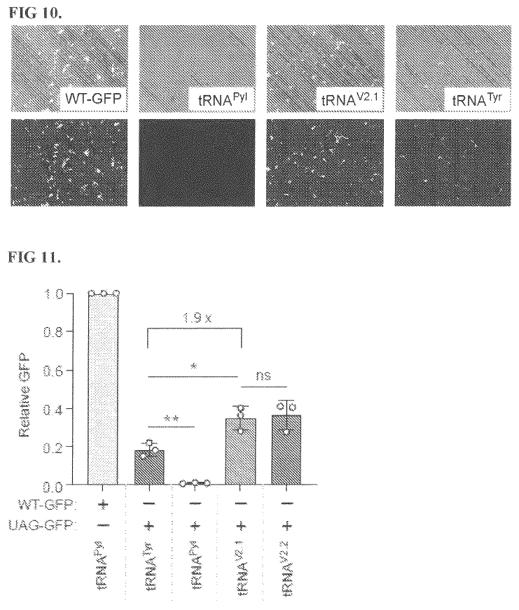

FIG. 10 shows fluorescence imaging of cells expressing WT-GFP or a UAG PTC mutant gene in the presence of an engineered tRNA^{Pyl} comparator, tRNAV2.1, or tRNAV2.2. While GFP expression was observed for each group treated with a chimeric tRNA, indicating UAG codon readthrough and facilitation of GFP protein synthesis, GFP expression was absent in cells treated with the previously described M. alvus tRNA^{Pyl} molecule. These data indicate that the acceptor stem mutations introduced into M. alvus tRNA^{Pyl} were essential for recognition of the engineered sup-tRNAs by the human AlaRS, which thereby enables PTC suppression in human cells.

For comparative purposes, a previously reported sup-tRNA based on human tyrosine tRNA (sup-tRNA^{Tyr}) was also evaluated in the GFP assay. Quantifying GFP expression confirmed that the two exemplary suppressor tRNAs, tRNAV2.1 and tRNAV2.2, restored GFP expression levels to ˜35% of levels observed from cells expressing the wild-type GFP gene, as shown in FIG. 10. Notably, the disclosed tRNAs were superior to sup-tRNA^{Tyr}. Although sup-tRNA^{Tyr} was previously shown to rescue PTCs in a mouse model of a human disease (Wang et al., Nature, 2022. 604 (7905): p. 343-348). FIG. 11 shows that exemplary disclosed tRNAs tRNAV2.1 and tRNAV2.2 displayed nearly twice the suppression efficiency of sup-tRNA^{Tyr}. Additionally, tandem mass spectrometry of GFP produced using tRNAV2.1 confirmed that alanine was incorporated at the position encoded by the UAG PTC (data not shown).

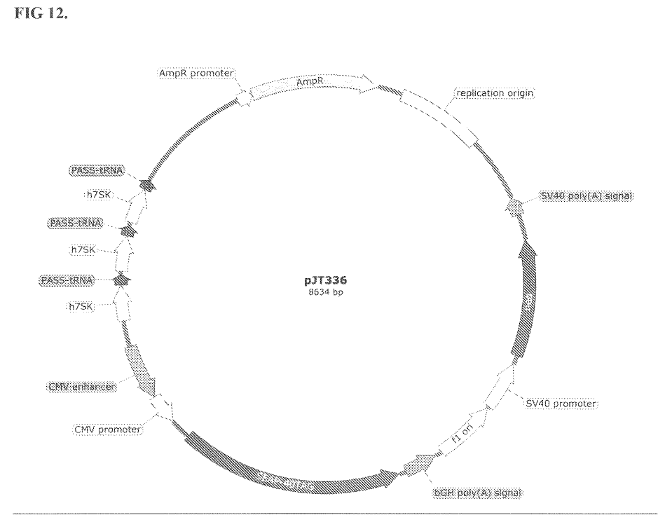

PTC Suppression in SEAP: In addition to GFP, the gene encoding secreted embryonic alkaline phosphatase (SEAP) was used as an alternative reporter to further establish the suppression capacity of tRNAV2.1 and tRNAV2.2. The SEAP gene was altered to include a UAG PTC mutation at position 40. An exemplary plasmid used to co-express SEAP and a disclosed sup-tRNA (PASS-tRNA) is shown in FIG. 12. (SEQ ID NOs: 26 and 27 show the CMV promoter and CMV enhancer; SEQ ID NO:29 show the BGH Poly-A signal for the expression cassette of FIG. 12). In the assay, SEAP expression was indicative of PTC suppression by the engineered tRNAs, such as by reading through the UAG stop codon and incorporating alanine into the growing amino acid chain.

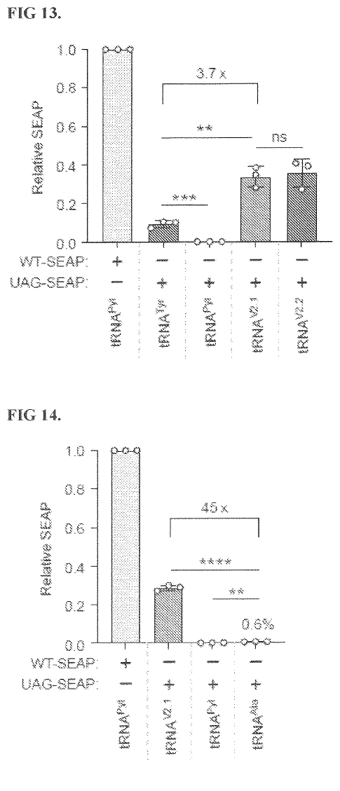

Consistent with the GFP-based results, FIG. 13 shows that tRNAV2.1 and tRNAV2.2, but not the previously described mutant tRNA^{Pyl} molecule, facilitated robust SEAP production. These data indicate that the chimeric M. alvus tRNAs suppress the UAG PTC in the SEAP gene. The disclosed chimeric tRNA^{Pyl} molecules restored SEAP expression to ˜34% of the expression observed from wild-type SEAP, which lacks a PTC. In these experiments, PTC suppression of disclosed chimeric tRNA^{Pyl} molecules was approximately 4-fold greater than the previously reported benchmark sup-tRNA^{Tyr}. These results, together with those shown in FIG. 11, confirm that the engineered M. alvus tRNAs are more efficient at suppressing PTCs than the previously reported sup-tRNA^{Tyr} molecule. Both exemplary tRNA^{Pyl} molecules were nearly 2- and 4-fold more efficient than sup-tRNA^{Tyr} in the GFP and SEAP reporters, respectively. Moreover, sup-tRNA^{Tyr} showed different readthrough efficiency in each reporter, which indicates that its suppression efficiency may be more influenced by the mRNA context in which the PTC is present.

To explore whether the suppression efficiency of the exemplary chimeric tRNA^{Pyl} molecules was due to the tRNA^{Pyl} scaffold, PTC suppression of a human alanine tRNA variant mutated to translate the UAG stop codon (sup-tRNA^{Ala}) was measured. Interestingly, sup-tRNA^{Ala} showed very weak UAG suppression, restoring less than 1% of WT SEAP expression. Although sup-tRNA^{Ala} and disclosed chimeric M. alvus tRNA^{Pyl} molecules are both aminoacylated by the human AlaRS enzyme, the chimeric tRNA^{Pyl} molecules were able to suppress a PTC in the SEAP reporter gene 45-fold more efficiently than sup-tRNA^{Ala}, such as V2.1, as shown in FIG. 14. Surprisingly, the suppression efficiency of the chimeric M. alvus tRNA^{Pyl} molecules described herein was comparable in both GFP and SEAP reporters, indicating that the engineered tRNAs can translate UAG codons with similar efficiency in different mRNA coding contexts.

### Example 3: Codon Specificity of Chimeric Suppressor M. Alvus tRNA^{Pyl} Molecules

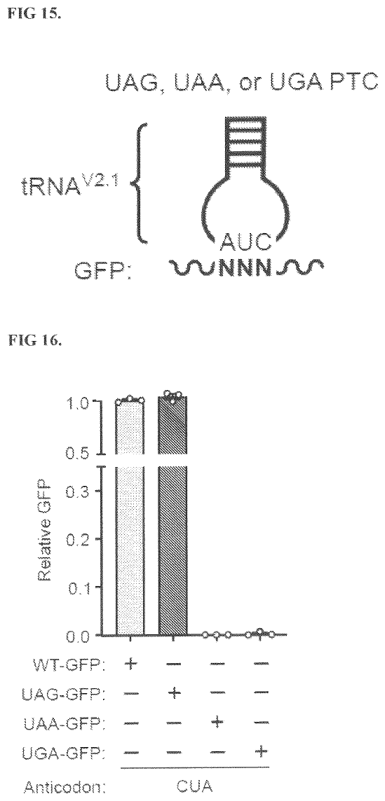

Near-cognate stop codon readthrough has been observed for suppressor tRNAs. See, e.g, Tharp et al., ACS Chem Biol, 2021; 1:766-77, Morosky et al., Front Mol Biosci, 2023; 10:1096261, and Brenner & Beckwith, Journal of Molecular Biology, 1965; 13:629-637. Such readthrough can have undesirable side effects. To evaluate whether the suppressor M. alvus tRNA^{Pyl} molecules described herein have off-target decoding activity, the ability of tRNA V2.1 to decode near-cognate stop codons was tested. This was achieved by measuring sfGFP expression in cells co-expressing tRNA V2.1 and a GFP mutant gene with UAA or UGA PTC at position 2, as depicted in FIG. 15. (SEQ ID NO: 31). Notably, co-expression of tRNA V2.1 with the GFP reporters containing UAA and UGA codons in E. coli cells did not yield GFP synthesis, as shown in FIG. 16. These results demonstrate that M. alvus tRNA^{Pyl} molecules engineered to base pair with the UAG stop codon, such as having a CUA anticodon, translate the UAG stop codon with high specificity.

### Example 4: Suppression of the UAA and UGA Premature Termination Codons by Chimeric Suppressor M. Alvus tRNA^{Pyl} Molecules in Human Cells

While the studies presented in Example 2 show effective suppression of UAG PTC in human cells by disclosed tRNA^{Pyl} molecules, the PTCs UGA and UAA are also associated with human genetic diseases. Therefore, sup-tRNAs that suppress UGA and UAA PTCs are also critically needed.

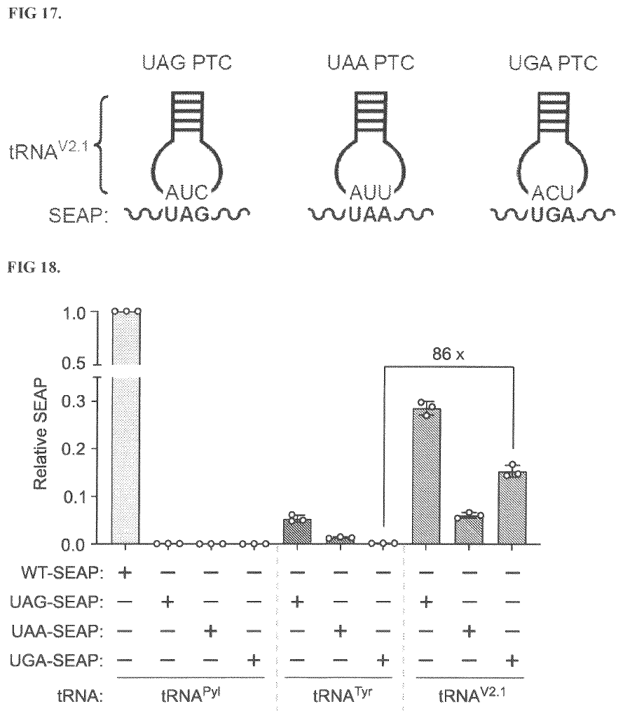

To test whether the engineered M. alvus sup-tRNAs could also be used to suppress UGA and UAA PTCs, the anticodon of tRNA V2.1 was mutated to translate UGA and UAA codons, as depicted by FIG. 17. Chimeric tRNAV2.1 molecules engineered to recognize the different stop codons were then co-expressed in HEK 293 cells along with a WT-SEAP reporter gene or a SEAP reporter gene having a UAG, UAA, or UGA PTC at position 40 (SEQ ID NO: 33). For comparison, the anticodons of the previously reported tRNA^{Pyl} and sup-tRNA^{Tyr} molecules were also mutated to facilitate recognitions of the various PTCs.

FIG. 18 shows SEAP expression in cells expressing anticodon mutants of tRNA^{Pyl}, tRNA^{Tyr}, or tRNA V2.1 relative to WT SEAP. While PTC readthrough was most efficient with the tRNA V2.1 molecule engineered to recognize the UAG stop codon, the other anticodon variants of tRNA V2.1 also afforded significant readthrough of both UAA and UGA. For example, tRNA V2.1 mutant bearing a UUA anticodon restored GFP expression to ˜6% of WT GFP, while tRNA V2.1 mutant harboring a UCA anticodon restored GFP expression to >15% that of the wild-type gene. The previously described tRNA^{Pyl} molecule bearing the corresponding anticodon mutations was unable to suppress either UGA or UAA PTCs in HEK 293.

The results demonstrate that the chimeric tRNA V2.1 anticodon variants robustly suppress all three stop codons in the SEAP reporter gene. In contrast, the anticodon mutants of the comparator tRNA^{Pyl} molecule did not suppress any of the stop codons in HEK cells, demonstrating the importance of the mutations introduced into exemplary disclosed M. alvus tRNA^{Pyl} molecules, with respect to their PTC suppression activity.

Furthermore, FIG. 18 shows that the tRNAV2.1 anticodon variants were more efficient than the benchmark sup-tRNA^{Tyr} anticodon variants at suppressing all three types of PTCs. While mutant sup-tRNA^{Tyr} was able to suppress both UGA and UAA, the suppression efficiency was low. Anticodon mutants of tRNA V2.1 suppressed UGA and UAA PTCs 86- and 4.6-fold more efficiently than sup-tRNA^{Tyr} mutants, respectively. The poor suppression capabilities of tRNA^{Tyr} anticodon mutants could be due to reduced aminoacylation efficiency since the anticodon is a recognition element for TyrRS. See, e.g., Giege & Eriani, Nucleic Acids Res, 51, 1528-1570. However, the superior suppression efficiency of tRNA V2.1 might also be attributable to the unique features within the tRNA^{Pyl} scaffold Together, these data demonstrate that exemplary disclosed suppressor tRNA molecules have the capacity to efficiently suppress all three PTCs in human cells.

### Example 5: Chimeric Suppressor M. Alvus tRNA^{Pyl} Molecules do not Significantly Decrease Cell Viability

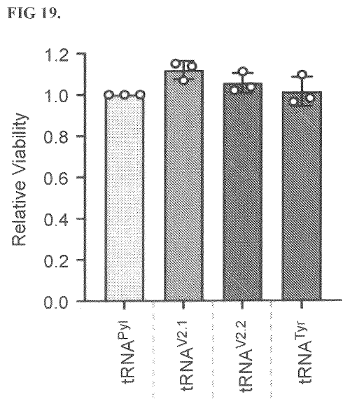

The expression of highly efficient sup-tRNAs may be toxic to cells due to readthrough of native termination codons. In order to assess the impact of suppressor M. alvus tRNA^{Pyl} molecules on cell viability, an MTT assay was used to evaluate cellular metabolic activity upon sup-tRNA expression. As shown in FIG. 19, HEK 293 cells expressing either exemplary disclosed chimeric tRNA^{Pyl} molecules tRNAV2.1 or tRNAV2.2, showed no significant decrease in viability compared to cells expressing a previously reported engineered tRNA molecule, either tRNA^{Pyl} or tRNA^{Tyr}, over 48 h. The previously described M. alvus tRNA^{Pyl} molecule was used as a negative control for this assay because experiments with the GFP and SEAP reporters demonstrated that this tRNA has no detectable UAG suppression capability in HEK 293. These data indicate that expressing the disclosed chimeric sup-tRNA molecules in human cells is not associated with reduced viability.

### Example 6: Sup-tRNAs Rescue Stop Codon Mutations in Tumor Suppressor p53

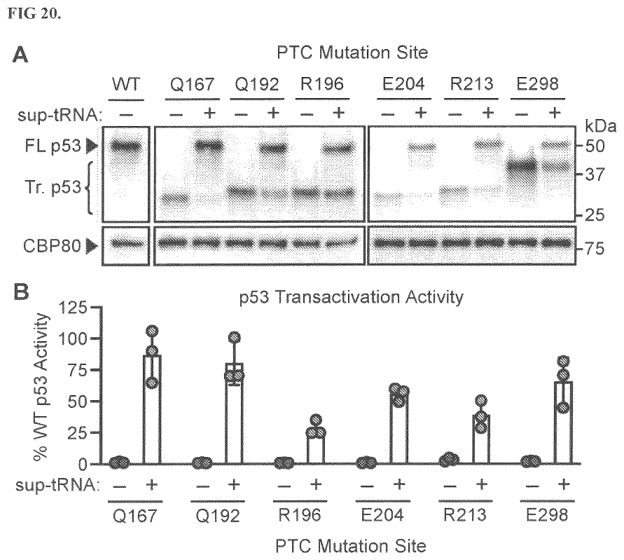

Sup-tRNAs translate PTCs and restore p53 synthesis from cDNA: To investigate whether sup-tRNAs can rescue p53 protein from genes harboring PTCs, from SEQ ID NO: 34, we generated TP53 cDNA harboring six cancer-associated PTC mutations. Our exciting results show that co-expression of sup-tRNA (SEQ ID NOs: 1, 2, 3 and 4) in p53-null NCI-H1299 cells transfected with cDNA plasmids restored the synthesis of full-length p53 from six PTCs (FIG. 20A). Importantly, the sup-tRNA-rescued p53 proteins are functionally active in transcription regulation, as demonstrated by a common luciferase-based transactivation assay (FIG. 20B).

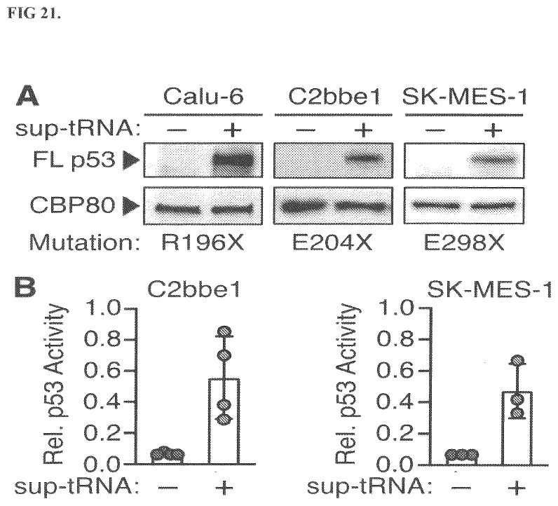

Sup-tRNAs rescue functional p53 in cells harboring endogenous PTCs: While experiments with TP53 cDNA are convenient for measuring PTC translation, they lack the genomic context and native TP53 promoter, limiting their biological relevance. To assess whether sup-tRNAs can suppress PTCs in native TP53, we utilized a colorectal cancer line, C2bbel, carrying homozygous E204 (TAG) mutations and two lung cancer lines, Calu-6 and SK-MES-1, carrying homozygous R196 (TGA) and E298 (TAG) mutations, respectively. Cells were transfected with a sup-tRNA-encoding plasmid and then p53 protein levels and luciferase transactivation were quantified as described above. Our data show that sup-tRNAs efficiently rescued functional p53 in all three cell lines (FIG. 21A-B)

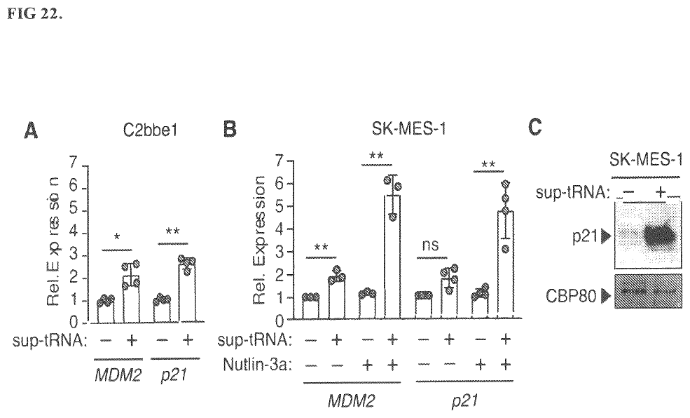

Sup-tRNAs synergize with Nutlin-3a to activate the p53 pathway: To further assess the activity of sup-tRNA-rescued p53, we used qRT-PCR to quantify expression of downstream genes in the p53 pathway, namely MDM2 and CDKN1A (p21), in C2bbel and SK-MES1 cells. In both cell lines, sup-tRNA transfection yielded significant increases in MDM2 mRNA, whereas a significant increase in p21 was only observed for C2bbel (FIG. 22A-B). Notably, sup-tRNA transfection had no impact on gene expression in p53-null NCI-H1299 cells (data not shown). To evaluate whether sup-tRNAs can synergize with small-molecule activators of p53, we treated SK-MES-1 cells with Nutlin-3a following sup-tRNA transfection, and then quantified MDM2 and p21 expression. Nutlin-3a is a small molecule and MDM2 (murine double minute 2) inhibitor, with potential antineoplastic activity. In cancer cells, Nutlin-3a antagonizes the binding of MDM2 to p53, thereby preventing MDM2-mediated p53 degradation. This results in stabilizing and activating p53-dependent cell cycle arrest and apoptosis. The protein MDM2, a negative regulator of p53 activity, is overexpressed in many cancer cell types. The combined treatment of sup-tRNA and Nutlin-3a led to greater increases MDM2 and p21 mRNA compared to either treatment alone (FIG. 22B). Increased p21 protein was further confirmed by immunoblotting (FIG. 22C). Collectively, these data demonstrate that sup-tRNAs can synergize with small molecules to reactivate the p53 pathway in cells harboring endogenous PTC mutations.

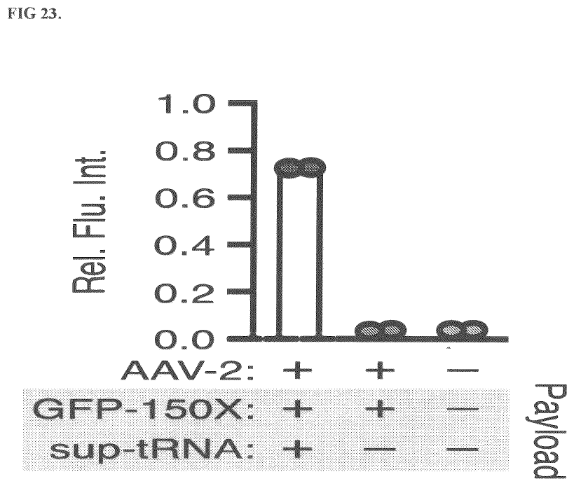

Sup-tRNAs can be delivered to cancer cells using adeno-associated viral vectors (AAVs): To determine if AAVs serve as efficient delivery vehicles, we cloned three copies of the sup-tRNA encoding gene, under control of a h7SK promoter, into an AAV-2 vector. Viral particles were produced and purified and then used to transduce NCI-H1299 lung cancer cells that were co-transfected with a reporter plasmid harboring GFP gene with a PTC at position 150. Thus, GFP fluorescence should only result from suppression of the PTC by our engineered sup-tRNA. We used flow cytometry to quantify GFP fluorescence in cells treated with an empty AAV or a sup-tRNA AAV. The data revealed GFP expression only in cells treated with a sup-tRNA that expresses the engineered sup-tRNA (FIG. 23), demonstrating activity of the AAV-delivered tRNA.

### Example 7: Sup-tRNAs Rescue Stop Codon Mutations in the Tumor Suppressor BRCA1

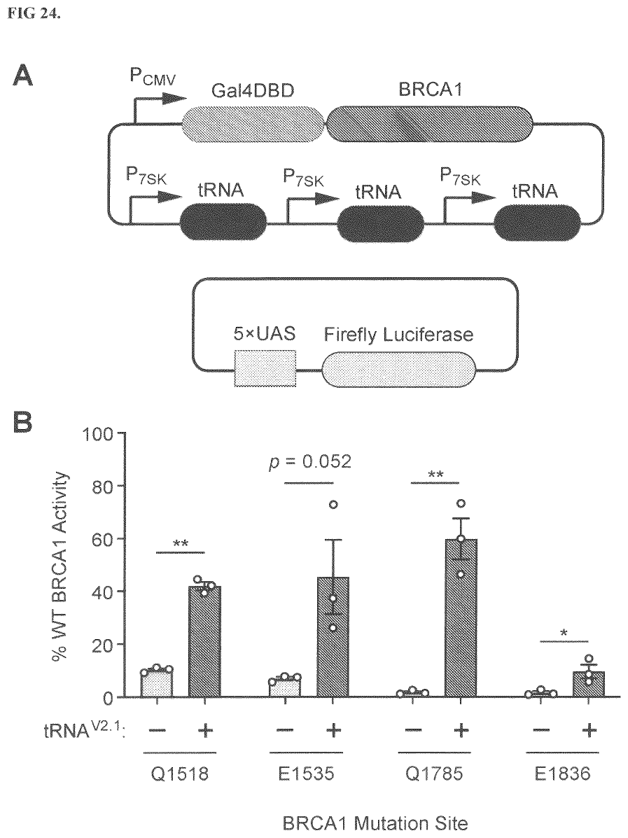

Next, we investigated whether sup-tRNAs could suppress pathogenic PTCs to restore synthesis of functional BRCA1 protein. Nonsense mutations within the BRCA1 gene significantly increase the risk of developing several types of cancer, principally breast and ovarian cancer (2). We measured suppression by tRNA^{V2.1} (SEQ ID NOs: 1 and 2) of four clinically relevant PTCs occurring at codons Q1518, E1535, Q1785, and E1836. These mutations were chosen, in part, because they were previously shown to tolerate amino acid substitutions without substantially impacting BRCA1 activity (3). The Gal4 DNA-Binding Domain (1-100)-BRCA1 (1396-1863) SEQ. ID NO: 35 was utilized in these experiments. As shown in FIG. 24A, Human BRCA1 (residues 1396-1863) containing an in-frame UAG PTC was expressed as a genetic fusion to the Gal4 DNA-binding domain (Gal4DBD). Three copies of tRNA V2.1 were expressed from the same plasmid. The Gal4DBD-BRCA1 plasmid was co-transfected with a plasmid containing the firefly luciferase (Fluc) gene and five copies of the Gal4 upstream activation sequence (5×UAS). We utilized a previously reported assay for measuring BRCA1-mediated transactivation of a luciferase reporter gene in HEK 293 cells with and without co-expression of tRNA^{V2.1} and compared the activity to that of WT BRCA1 (without a PTC).

With each nonsense variant, cells expressing tRNA^{V2.1} displayed higher BRCA1 activity than cells not expressing a sup-tRNA (FIG. 24B); however, the level of BRCA1 activity varied. For the Q1518, E1535, and Q1785 mutants, BRCA1 activity was restored to between 42 and 60% of that of cells expressing WT BRCA1, whereas with the E1836 mutant, BRCA1 activity was restored to less than 10% of WT. This discrepancy may be due to differences in suppression efficiency of PTCs in different sequence contexts. The background activity of each of the PTC mutants also varied significantly, suggesting that sequence context contributes to background suppression. An alternative explanation is that the E1836 variant does not tolerate alanine substitution as well as the other mutants. Nonetheless, these data demonstrate that tRNA V2.1 can suppress pathogenic PTCs within BRCA1 to restore functional BRCA1 protein.

### Example 8: Sup-tRNA Safety

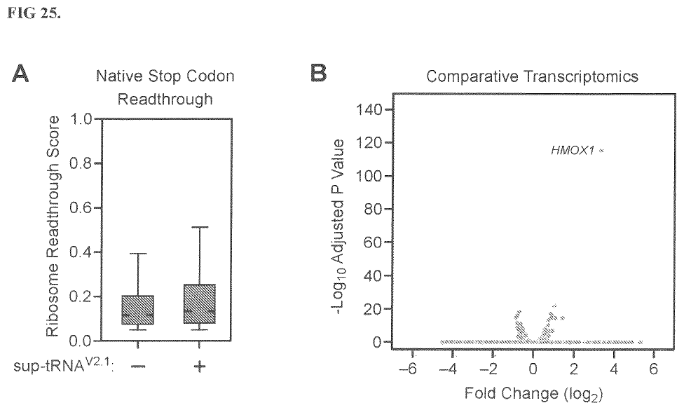

PASS-tRNA V2.1 Minimally Suppresses Native Stop Codons: A key safety consideration with respect to the therapeutic use of sup-tRNAs is their potential for translating natural termination codons. Off-target translation of native stop codons would cause the synthesis of proteins with extended C-termini that could dysregulate the proteome. To determine the extent to which PASS-tRNAs disrupt translation termination of native genes, we used ribosome profiling to quantify global stop codon readthrough in HEK 293 cells transfected with a plasmid expressing tRNA^{V2.1} and compared the level of stop codon readthrough to cells transfected with an empty vector. Our calculated ribosome readthrough scores (RRTS), which measure the density of ribosomes in the 3′-UTR, show only a minor increase in readthrough of native stop codons relative to control cells not expressing tRNA^{V2.1} (RRTS=0.07 and 0.09 for control and treated cells, respectively; FIG. 25A). These results indicate that tRNA^{V2.1} does not significantly increase readthrough of native termination codons, in agreement with several recent studies showing minimal native stop codon readthrough elicited by sup-tRNAs.

PASS-tRNA^{V2.1} Does Not Alter Global Gene Transcription: To further investigate potential undesired effects of PASS-tRNAs, we performed a comparative transcriptomic analysis using total mRNA from HEK 293 cells harboring an empty vector or a vector expressing tRNA V2.1. We found that expressing PASS-tRNA^{V2.1} in HEK 293 cells does not elicit significant changes in the cellular transcriptome (FIG. 25B), suggesting that the tRNA does not interfere with transcriptional homeostasis or detectably trigger off-target pathways. The only significantly upregulated gene in PASS-tRNA-expressing cells relative to control cells was HMOX1, encoding Heme Oxygenase 1, an enzyme involved in iron metabolism. Further studies are required to better understand the mechanistic rationale for HMOX1 upregulation and its potential biological significance. However, the cell viability and gene expression data collectively support recent studies demonstrating low toxicity of sup-tRNAs in human cells.

### Materials & Methods

Cloning and Sequencing: Molecular cloning was performed using HiFi DNA Assembly (New England Biolabs or In-Fusion Cloning (Takara) and E. coli Stellar cells (Takara). Synthetic oligonucleotides and gene fragments were purchased from Integrated DNA Technologies (IDT), the Keck Oligonucleotide Synthesis Facility at Yale University, and Sigma. DNA sequencing services were provided by GeneWiz, Quintarabio, and Keck DNA Sequencing Facility at Yale University. Mass spectrometry analysis of GFP expressed in HEK 293 was provided by the Indiana University School of Medicine Center for Proteome Analysis.

Plasmid Construction: Plasmids expressing Methanomethylophilus alvus and Methanosarcina mazei tRNA molecules and super-folder green fluorescent protein (sfGFP) (WT or with a termination codon at position 2) genes have been reported previously (Tharp et al., ACS Chem Biol, 16, 766-774.). The tRNAs are constitutively expressed under an lpp promoter, while sfGFP is controlled by an arabinose-inducible promoter. tRNA anticodon variants and sfGFP variants containing different stop codons were generated by site-directed mutagenesis. For experiments in HEK 293 cells, the previously reported M. alvus tRNA^{Pyl} gene with an h7SK promoter was synthesized by IDT and cloned into an empty pBAD plasmid. To generate the tRNA variants, this template plasmid was modified by site-directed mutagenesis. To clone expression vectors with three copies of the tRNAs, h7SK-tRNA fragments were amplified with three primer sets having unique adapter sequences. The products of these PCRs were then simultaneously cloned into pCDNA3.1 receptor plasmids, encoding SEAP-40TAG (Jiang et al., Proc Natl Acad Sci USA, 120, e2219758120.) (SEQ ID NO:32) or GFP-150TAG (Meineke et al., ACS Chem Biol, 13, 3087-3096) (SEQ ID NO:28), using NEBuilder® HiFi DNA Assembly. All plasmid constructs were confirmed by Sanger sequencing.

Fluorescence-based Assay in E. coli: E. coli BW25113 or MG1655 cells were transformed with pBAD plasmids encoding a PTC-containing sfGFP and one of the tRNA variants. Fresh colonies were used to inoculate 1 mL of LB media supplemented with ampicillin (100 μg/mL), and cells were grown overnight with continuous shaking at 37° C. 0.5 μL of the overnight culture was used to inoculate 99.5 μL LB media containing ampicillin (100 μg/mL) in the absence or presence of 0.15% arabinose in a 96-well black, clear-bottom plate. Cell density (OD600) and sfGFP fluorescence intensity (lex=485 nm, lem=510 nm) were monitored for 24 h with constant shaking at 37° C. using a BioTek microplate reader (HTX or synergy H1). Data from the 24 h time point are reported. To remove the background signal, the OD600 and fluorescence intensity values from uninduced cells were subtracted from the same values from cells induced with arabinose. Readthrough efficiency was calculated by dividing sfGFP fluorescence by OD600, and data were plotted using Prism 9 (GraphPad).

Purification and Mass Spectrometry Analysis of sfGFP from E. coli: Chemically competent E. coli MG1655 cells were transformed with a pBAD plasmid encoding either WT sfGFP with M. alvus tRNA V2.1, sfGFP-S2TAG with tRNA V2.1, or sfGFP-S2TAG with tRNA V2.2. Fresh colonies were used to inoculate 2 mL of LB media with ampicillin (100 μg/mL). Cells were grown overnight under continuous shaking at 37° C. The starter cultures were used to inoculate 50 mL of LB media with ampicillin, and the cell cultures were grown with continuous shaking at 37° C. to an OD600 of 0.7-1. sfGFP expression was induced with 0.2% arabinose overnight. The cultures were centrifuged at 6000 rpm for 15 min at 4° C. Cell pellets were resuspended with 750 μL lysis buffer (50 mM Tris pH 8, 300 mM NaCl, 10 mM imidazole, and 1× BugBuster protein extraction reagent, Sigma). The lysed cells were centrifuged at 13,000 rpm for 35 min at 4° C., and the lysate was loaded on a column containing 0.6 mL TALON resin, prewashed with 5 mL of deionized water and 3 mL of buffer containing 50 mM Tris pH 8, 150 mM NaCl, and 10 mM imidazole. sfGFP was eluted using 1.5 mL of elution buffer (50 mM Tris pH 8, 300 mM NaCl, and 300 mM imidazole). Eluted protein was concentrated using a 10 kDa centrifugal filter Unit (Millipore), and the proteins were stored in 50 mM Tris pH 8, 150 mM NaCl. An aliquot containing 25 μg of purified sfGFP was diluted with 0.1 M ammonium bicarbonate in water (pH 8) and reduced and alkylated at room temperature using 5 mM dithiothreitol for 1.5 h, followed by 10 mM iodoacetamide for 45 min in the dark. Prior to the addition of protease, 100 mM CaCl2 was added to yield a final concentration of 10 mM. Proteolysis was achieved using sequencing grade chymotrypsin (Promega Corporation P/N V1062) at an enzyme: protein ratio of 1:20 (w/w) and proceeded for 6 hr at 37° C. Digestion was quenched using formic acid to yield a final pH of 2.5. Chymotryptic peptides were desalted using reversed-phase spin columns (Pierce™ P/N 89873) following the manufacturer's instructions. Desalted peptides were dried to completion, resuspended in 0.1% formic acid in water, and quantified by measuring the absorbance at 280 nm on a NanoDrop spectrophotometer (Thermo Scientific). An aliquot containing 160 ng of peptides from each digestion was subjected to a 1 hr reversed phase Ultra-high Performance Liquid Chromatography (UPLC) gradient using a Thermo Scientific Ultimate 3000 RSLCnano UPLC instrument. Peptides were eluted directly from the Waters nanoEase m/z Peptide BEH C18 analytical column (Waters Corporation P/N 186008795) into an Orbitrap Eclipse Tribrid mass spectrometer (Thermo Scientific) operated in positive electrospray mode using a 3 sec maximum cycle time, TopN data-dependent acquisition method for tandem mass spectrometry (MS/MS) acquisition. MS and MS/MS scans were acquired at high resolution in the Orbitrap mass analyzer. Peptide and protein identifications were generated by searching all raw UPLC-MS/MS raw files against the E. coli strain BL21 (DE3) Uniprot annotated proteome database (identifier UP000002032, accessed Jan. 23, 2023) plus a custom sfGFP database including WT sfGFP with all 20 standard amino acids in position 2, using the Andromeda search engine embedded in Max Quant v1.6.10.43 (Cox & Mann, Nat Biotechnol, 26, 1367-1372.). Variable modifications included oxidation of Met, acetylation of the protein N-terminus, deamidation of Asn/Gln, and peptide N-terminal Gln to pyro-Glu conversion. Carbamidomethylation of Cys was set as a fixed modification, and enzyme specificity was set to “unspecific.” A 1% false discovery rate was used at the peptide-spectrum match (PSM), and protein levels and a minimum number of amino acids for an unspecific search were set to 6. All other parameters were left at default values. Search results were uploaded into Scaffold v5 (Proteome Software, Inc.) for data visualization and additional analysis. For label-free quantitative analysis of amino acids incorporated at residue 2, a 5 ppm mass window was used to generate extracted ion chromatograms in Xcalibur (Thermo Scientific) using theoretical monoisotopic masses for peptide sequences identified by MaxQuant. All charge states within the MS mass range were used for peak integrations. Stoichiometry estimates for residue 2 amino acids were not corrected to account for differences in ionization efficiency.

HEK 293 Cell Culture: HEK 293 cells were purchased from ATCC (CRL-3216). Cells were cultured in Dulbecco's Modified Eagle Medium (DMEM) with glutamine XL (Neta Scientific) supplemented with 10% fetal bovine serum (FBS; Gibco) and penicillin-streptomycin (Gibco). Cells were maintained in a humidified incubator at 37° C. with a 5% CO2 atmosphere.

Suppression Assay with SEAP Reporter in HEK 293 Cells: HEK 293 cells were seeded in a 24-well plate and transfected with 0.5 ug of plasmid DNA using 3 μL FuGene6 (Promega) according to the manufacturer's protocol. Approximately 24 and 48 h after transfection, 25 μL of media was removed from each well, and the secreted embryonic alkaline phosphatase SEAP) activity was quantified using the Phospha-Light™ SEAP Reporter Gene Assay System (Applied Biosystems) according to the manufacturer's protocol. Luminescence was measured in white, flat-bottom, 96-well plates using a BioTek Synergy H1 microplate reader (0.02 s/well). Data were normalized by dividing the luminescence signal of each sample by the signal of the positive control (SEAP with no PTC) and displayed as the mean±SEM of three biological replicates.

Suppression Assay with sfGFP Reporter in HEK 293 Cells: For sfGFP quantification, HEK 293 cells were seeded in a 6-well plate and transfected with 2 ug plasmid DNA using 12 μL FuGene6 (Promega) according to the manufacturer's protocol. Approximately 48 h after transfection, the media was removed, and the cells were washed twice with PBS (Gibco). Cells were lysed for 20 min on ice using 150 μL of RIPA Lysis and Extraction Buffer (Thermo Scientific) supplemented with protease inhibitor cocktail (Thermo Scientific). Following lysis, the insoluble fraction was removed by centrifugation, and the supernatant was transferred to a new tube. Total protein content was measured using the Pierce™ BCA Protein Assay Kit according to the manufacturer's protocol. sfGFP fluorescence (λex=485 nm, λem=528 nm) was quantified using 100 μL of cell lysate in black, clear-bottom 96-well plates in a BioTek Synergy H1 microplate reader. Relative sfGFP expression values were calculated by dividing the sfGFP fluorescence signal by the total protein content for each sample, as described previously (Meineke et al., ACS Chem Biol, 13, 3087-3096). Data were normalized by dividing each sample's relative GFP expression fluorescence by the positive control fluorescence (GFP without a PTC mutation). Data are displayed as the mean±SEM of three biological replicates. For fluorescence imaging, cells were seeded in a 96-well plate and transfected with 0.1 ug of plasmid DNA using polyethyleneimine (PEI) with a DNA: PEI ratio of 1:4. Cell images were collected approximately 48 h after transfection using an EVOS M5000 microscope.

Cell Viability Assay: The viability of HEK 293 cells expressing various sup-tRNAs was measured using the MTT assay (Cayman Chemical) according to the manufacturer's protocol. Briefly, ˜20,000 viable cells (in 100 μL of media) were seeded in each well of a 96-well plate. The following day, cells were transfected with the appropriate plasmids using FuGene6 (Promega) according to the manufacturer's protocol. Approximately 40 h after transfection, 10 μL MTT reagent was added to each well, and the media was mixed. After an additional 3 h, 100 μL of solubilization buffer was added, and each well was vigorously mixed. Absorbance at 570 nm was measured using a BioTek Synergy H1 microplate reader after incubating the plates for an additional 2 h at 37° C. Data are displayed as the mean±SEM of three biological replicates.

sfGFP Mass Spectrometry: HEK 293 cells were seeded in a 60 mm dish and transfected with 5 μg of plasmid DNA using 30 μL of FuGene6 (Promega) according to the manufacturer's protocol. Approximately 48 h after transfection, the media was removed, and the cells were washed twice with PBS (Gibco) before being lysed on ice for 20 min using 0.5 mL RIPA Lysis and Extraction Buffer (Thermo Scientific) supplemented with protease inhibitor cocktail (Thermo Scientific). Following lysis, the insoluble fraction was removed, and the soluble lysate (500 μL) was diluted with 200 μL of dilution buffer (10 mM Tris pH 7.5, 150 mM NaCl, 0.5 mM EDTA) supplemented with protease inhibitor cocktail. The diluted lysate was mixed with 25 μL of ChromoTek GFP-Trap® Magnetic Agarose beads prewashed with dilution buffer. The beads-lysate mixture was incubated at 4° C. for one hour with end-over-end rotation. The protein-bound beads were washed three times with 0.5 mL wash buffer (10 mM Tris pH 7.5, 150 mM NaCl, 0.05% NP-40, 0.5 mM EDTA) and once with dilution buffer. After the final wash, the beads were resuspended in 20 μL dilution buffer and stored at −80° C. prior to mass spectrometry.

For mass spectrometry analysis, protein-bound beads were resuspended in 8 M urea and reduced with 5 mM TCEP at 35° C. for 30 min. Following TCEP reduction, the sample was alkylated with 10 mM chloroacetylchloride for 30 min at room temperature. The alkylated sample was digested with 0.5 ug trypsin/LysC at 35° C. overnight, and the digested sample was desalted using a C18 spin column. One half of the sample was injected using an EasyNano 1200 (Thermo Fisher Scientific) with a 25 cm EasySpray column (Cat. No. ES902, Thermo Fisher Scientific) and a 90 min LC gradient (hold at 8% B for 5 min, 8-35% B over 70 min, 35-80% B over 9 min, hold at 80% B for 1 minute, 80-4% B over 5 min; Buffer A: 0.1% formic acid in water, Buffer B: 0.1% formic acid, 80% acetonitrile, 20% water). An Exploris 480 with FAIMS pro (Thermo Fisher Scientific) was used for the MS/MS analysis with FAIMS CVs of −40, −55, and −70 V, each for 1.3 s cycle times. MS1 settings: orbitrap resolution of 120000, RF lens 40%, standard AGC target, auto max IT, charge states of 2-7, dynamic exclusion 30 sec. MS2 settings of 1.6 m/z isolation window, 30% HCD collision energy, orbitrap resolution of 15000, fixed first mass of 110 m/z, standard AGC target, and auto max IT. Data analysis was performed using PEAKS 11 (Bioinformatics Solutions) using the Homo sapiens Uniprot reference proteome (downloaded May 2022) supplemented with the GFP sequence and common laboratory contaminants. Following de novo peptide sequencing, a database search was performed with precursor mass tolerance of 10 ppm, fragment mass tolerance of 0.02 Da, semi-specific trypsin digest with a max of 3 missed cleavages, fixed modification of carbamidomethyl C, and variable modifications of deamidation on N and Q and oxidation on M. Database search was followed by PEAKS PTM and PEAKS Spider searches to look for over 300 PTMs and any potential mutations. Data were filtered for a 1% PSM FDR.

### BRCA1 Methods.

For measuring PTC readthrough in BRCA1, we employed a fluorescence-based transactivation assay described previously (1,2). Briefly, the yeast Gal4 DNA-binding domain (Gal4; residues 1-100) was cloned from plasmid Gal4-VP16 (a gift from Lea Sistonen; Addgene plasmid #71728; http//n2t.net/addgene: 71728; RRID: Addgene_71728) as a genetic fusion to human BRCA1 (residues 1396-1863) (a gift from Stephen Elledge; Addgene plasmid #14999; http://n2t.net/addgene: 14999; RRID: Addgene_14999) in a pCDNA3.1 plasmid harboring three copies of the gene encoding tRNA V2.1 under control of a 7SK promoter. PTCs were then introduced into the BRCA1 gene by site-directed mutagenesis. For measuring BRCA1 activity, HEK 293 cells were seeded in a white, clear-bottom 96-well plate and co-transfected with the Gal4-BRCA1 plasmid (22 ng), 5×Gal4-TATA-luciferase (87 ng, a gift from Richard Maurer; Addgene Plasmid #46756; http://n2t.net/addgene: 46756; RRID: Addgene_46756), and pLX313-Renilla (8.7 ng, a gift from William Hahn and David Root; Addgene plasmid #118016; http://n2t.net/addgene: 118016; RRID: Addgene_118016) using Lipofectamine™ 3000 according to the manufacturer's protocol. Fluc and Rluc activity were quantified 24 hours after transfection using the Dual-Glo® Luciferase Assay Kit (Promega) in a BioTek Synergy H1 microplate reader.

### Ribosome Profiling Library Preparation.

HEK 293 cells were seeded in a 100 mm plate and transfected with 10 μg of an empty pCDNA3.1 plasmid (negative control) or a pCDNA3.1 plasmid containing three copies of tRNA V2.1 using FuGene6 (Promega). All samples were prepared in biological duplicate. 48 h after transfection, total RNA and ribosome-protected fragments (RPFs) were prepared as described previously (3) with a few modifications. Briefly, following digestion with RNase I, samples were placed on 1.4 mL of a sucrose cushion (1 M sucrose, 20 mM Tris pH 7.4, 150 mM NaCl, 5 mM MgCl₂, 100 μg/mL cycloheximide, 20 U/mL SUPERase·INTM RNase inhibitor) and centrifuged at 4° C. for 2 hours at 60,000 rpm. The supernatant was discarded and RPFs were purified from ribosome pellets using the miRNeasy Mini Kit (Qiagen). Size-selection of RPFs and total RNA by gel electrophoresis, dephosphorylation, linker ligation, and subsequent purification were performed as described (4). Following purification, samples were pooled, and ribosomal RNA was depleted using the species-specific riboPOOL rRNA Depletion Kit (siTOOLS Biotech) according to manufacturer's protocol. 5′ adapter ligation, cDNA synthesis, and library amplification were performed as described previously (5). SuperScrip™ IV Reverse Transcriptase (Invitrogen) was used for reverse transcription. NEBNext Multiplexed Oligos (NEB) and Phusion™ High-Fidelity DNA Polymerase (Thermo Scientific) were used for library amplification. Following amplification, libraries were purified on an 8% TBE gel and sequenced by Azenta Life Sciences.

### Ribosome Profiling Analysis.

Paired-end sequencing (150-nt) was performed on an Illumina NovaSeq X Plus platform. Samtools was used to merge sequencing files for read 1 to produce one Fastq file per replicate. The Illumina TruSeq adapter sequence was trimmed from read 1 after aligning to the 3′-end, requiring a minimum global alignment score of 40 using ReadKnead v0.1.2 (6). After trimming adapters, the unique molecular identifier (UMI) was trimmed from the 3′ end and kept within the read name to identify PCR duplicates. Reads (only read 1) were aligned to the Ensembl 108 GRCh38 genome assembly using STAR v2.7.1a with the following nondefault parameters: --alignEndsType EndToEnd, --sjdbScore 2, --outFilterMultimapNmax 1000, outMultimapperOrder Random, --imitOutSAMoneReadBytes 600000. SAM files produced were converted to BAM files with samtools. Duplicates were removed with rmdup v1.3. Profile counts were generated with GeneAbacus v0.2.2 with a minimum read overlap of 10 with transcript (7).

Ribosome Readthrough Scores (RRTS) were calculated as described previously (8). One transcript was selected per protein-coding gene. These transcripts were selected based on i) complete coding sequences and UTRs, ii) with the most 3′ stop codon, and iii) sequentially preferring primary APPRIS transcripts (9), iv) inclusion in the consensus CDS gene set (CCDS) (10), and v) those with the longest coding sequence. If more filtering of transcripts per gene was required, transcripts with the shortest 3′-UTR and then shortest 5′-UTR were selected. These transcripts were further filtered by those with more than 200 reads in all samples. Ribosome footprint reads profiles were shifted by 13 nt to align the read start position to the ribosome P-site. The RRTS per transcript was calculated from the 3′-UTR ribosome density to the first termination codon in the 3′-UTR (3′TC) divided by the CDS ribosome density. The 3′-UTR ribosome density was calculated by summing the reads in the region 7nt after the CDS to the first 3′TC and dividing by the length of this region. Transcripts with less than 5 codons between the normal termination codon (NTC) and the first 3′TC were discarded. CDS densities were calculated by reads per length of CDS, excluding the first 18 nts and last 15 nts.

### RNA-Sequencing Analysis.

Paired-end sequencing 150-nt was performed on an Illumina NovaSeq X Plus platform. Samtools was used to merge sequencing files for read 1 to produce one Fastq file per replicate. The Illumina TruSeq adapter sequence was trimmed from read 1 after aligning to the 3′ end, requiring a minimum global alignment score of 40 using ReadKnead v0.1.2 (6). After trimming adapters, the unique molecular identifier (UMI) was trimmed from the 3′ end and kept within the read name to identify PCR duplicates. Reads (only read 1) were aligned to the Ensembl 108 GRCh38 genome assembly using STAR v2.7.1a with the following nondefault parameters: --alignEndsType EndToEnd, --sjdbScore 2, --outFilterMultimapNmax 1000, outMultimapperOrder Random, --imitOutSAMoneReadBytes 600000. SAM files produced were converted to BAM files with samtools v1.16.1. Duplicates were removed with rmdup v1.3. Counting was performed with GeneAbacus v0.2.2 with a minimum read overlap of 10 with transcript (7). Differential expression analysis was performed using DEseq2 v1.40.1 (11) with a parametric fit and custom R scripts.

### p53 Western Blot.

For measuring p53 protein expression by Western blot using TP53 cDNA, NCI-H1299 cells were grown in a 6-well plate in RPMI media supplemented with 10% FBS. Cells were transfected, using Fugene 6 transfection reagent, with 2 μg of a plasmid containing the gene encoding p53 with the indicated nonsense mutation. The plasmid also harbored three copies of a sup-tRNA as indicated. 24 hours after transfection, cells were lysed in RIPA lysis buffer for 30 minutes on ice and then clarified by centrifugation at 13,000 rpm for 15 minutes. Cell lysates (25 ug) were resolved by SDS-PAGE and then blotted onto a PVDF membrane. The membrane was blocked with 4% bovine-serum albumin. p53 protein was detected using anti-p53 antibody (sc-126; Santa Cruz Biotechnology, 1:500 dilution) and CBP80 protein was detected using anti-CBP80 antibody (sc-271304; Santa Cruz Biotechnology, 1:1000 dilution). Primary antibodies were probed with goat-anti-mouse-HRP conjugated antibody (31430; Thermo Scientific).

For measuring p53 protein expression by Western blot from genomically encoded TP53, SK-MES-1, Calu-6, or C2bbel cells were grown in a 6-well plate and then transfected with 2 μg of a plasmid encoding three copies of the indicated sup-tRNA gene. Cells were cultured an additional 48 hours. Cell lysates were prepared and analyzed by Western blot as described above. p53 Activity Assay.

For measuring p53 transactivation activity in NCI-H1299 cells, cells were transfected with a plasmid expressing TP53 cDNA, harboring the indicated nonsense mutations, with and without three copies of the indicated sup-tRNA encoding genes. Cells were co-transfected with pG13-Luc plasmid and a Renilla luciferase plasmid. Firefly and Renilla luciferase activity were quantified 24 hours after transfection using the Dual-Glo luciferase assay system (E2920; VWR). The same procedure was used for measuring p53 transactivation activity in established cell lines.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Sequence Table

| SEQ ID | | |
|---|---|---|
| NO. | Sequence | Description |
| 1 | GGGGGACGGUCCGGCGACCAGCGGGUCUCUAAAACCUA | Mutant M. |
| | GCAUAGCGGGGUUCGACACCCCGGUCUCUCG | alvus |
| | | tRNAPyl |
| 2 | GGGGGACGGUCCGGCGACCAGCGGGUCUCUAAAACCUA | M. alvus Sup |
| | GCAUAGCGGGGUUCGACACCCCGGUCUUCCA | tRNA V2.1- |
| | | CUA |
| | | anticodon |
| 3 | GGGGGACGGUCCGGCGACCAGCGGGUCUUUAAAACCUA | M. alvus Sup |
| | GCAUAGCGGGGUUCGACACCCCGGUCUUCCA | tRNA V2.1- |
| | | UUA |
| | | anticodon |
| 4 | GGGGGACGGUCCGGCGACCAGCGGGUCUUCAAAACCUA | Sup |
| | GCAUAGCGGGGUUCGACACCCCGGUCUUCCA | tRNA V2.1- |
| | | UCA |
| | | anticodon |
| 5 | GGGGGACGGUCCGGCGACCAGCGGGUCUCUAAAACCUA | M. alvus Sup |
| | GCAUAGCGGGGUUCGACACCCCGGUCCUCCA | tRNA V2.2- |
| | | CUA |
| | | anticodon |
| 6 | GGGGGACGGUCCGGCGACCAGCGGGUCUUUAAAACCUA | M. alvus Sup |
| | GCAUAGCGGGGUUCGACACCCCGGUCCUCCA | tRNA V2.2- |
| | | UUA |
| | | anticodon |
| 7 | GGGGGACGGUCCGGCGACCAGCGGGUCUUCAAAACCUA | M. alvus Sup |
| | GCAUAGCGGGGUUCGACACCCCGGUCCUCCA | tRNA V2.2- |
| | | UCA |
| | | anticodon |
| 8 | GGGGGACGGTCCGGCGACCAGCGGGTCTCTAAAACCTAG | Mutant M. |
| | CATAGCGGGGTTCGACACCCCGGTCTCTCG | alvus |
| | | tRNAPyl |
| 9 | GGGGGACGGTCCGGCGACCAGCGGGTCTCTAAAACCTAG | M. alvus Sup |
| | CATAGCGGGGTTCGACACCCCGGTCTTCCA | tRNA V2.1- |
| | | CUA |
| | | anticodon |
| 10 | GGGGGACGGTCCGGCGACCAGCGGGTCTTTAAAACCTAG | M. alvus Sup |
| | CATAGCGGGGTTCGACACCCCGGTCTTCCA | tRNA V2.1- |
| | | UUA |
| | | anticodon |
| 11 | GGGGGACGGTCCGGCGACCAGCGGGTCTTCAAAACCTAG | M. alvus Sup |
| | CATAGCGGGGTTCGACACCCCGGTCTTCCA | tRNA V2.1- |
| | | UCA |
| | | anticodon |
| 12 | GGGGGACGGTCCGGCGACCAGCGGGTCTCTAAAACCTAG | M. alvus Sup |
| | CATAGCGGGGTTCGACACCCCGGTCCTCCA | tRNA V2.2- |
| | | CUA |
| | | anticodon |
| 13 | GGGGGACGGTCCGGCGACCAGCGGGTCTTTAAAACCTAG | M. alvus Sup |
| | CATAGCGGGGTTCGACACCCCGGTCCTCCA | tRNA V2.2- |
| | | UUA |
| | | anticodon |
| 14 | GGGGGACGGTCCGGCGACCAGCGGGTCTTCAAAACCTAG | M. alvus Sup |
| | CATAGCGGGGTTCGACACCCCGGTCCTCCA | tRNA V2.2- |
| | | UCA |
| | | anticodon |
| 15 | CTGCAGTATTTAGCATGCCCCACCCATCTGCAAGGCATTC | h7SK |
| | TGGATAGTGTCAAAACAGCCGGAAATCAAGTCCGTTTAT | Promoter |
| | CTCAAACTTTAGCATTTTGGGAATAAATGATATTTGCTAT | |
| | GCTGGTTAAATTAGATTTTAGTTAAATTTCCTGCTGAAGC | |
| | TCTAGTACGATAAGTAACTTGACCTAAGTGTAAAGTTGA | |
| | GATTTCCTTCAGGTTTATATAGCTTGTGCGCCGCCTGGGT | |
| | ACCTC | |
| 16 | GAGGGCCTATTTCCCATGATTCCTTCATATTTGCATATAC | U6 Promoter |
| | GATACAAGGCTGTTAGAGAGATAATTAGAATTAATTTGA | |
| | CTGTAAACACAAAGATATTAGTACAAAATACGTGACGTA | |
| | GAAAGTAATAATTTCTTGGGTAGTTTGCAGTTTTAAAATT | |
| | ATGTTTTAAAATGGACTATCATATGCTTACCGTAACTTGA | |
| | AAGTATTTCGATTTCTTGGCTTTATATATCTTGTGGAAAG | |
| | GACGAAACACC | |
| 17 | GAACGCTGACGTCATCAACCCGCTCCAAGGAATCGCGGG | H1 Promoter |
| | CCCAGTGTCACTAGGCGGGAACACCCAGCGCGCGTGCGC | |
| | CCTGGCAGGAAGATGGCTGTGAGGGACAGGGGAGTGGC | |
| | GCCCTGCAATATTTGCATGTCGCTATGTGTTCTGGGAAAT | |
| | CACCATAAACGTGAAATGTCTTTGGATTTGGGAATCTTAT | |
| | AAGTTCTGTATGAGACCACTCTTTCCC | |
| 18 | TTTTT | Termination |
| | | Sequence |
| 19 | TTTTTT | Termination |
| | | Sequence |
| 20 | TTTTTTT | Termination |
| | | Sequence |
| 21 | TTTTTTTT | Termination |
| | | Sequence |
| 22 | TTTTTTTTT | Termination |
| | | Sequence |
| 23 | TTTTTTTTTT | Termination |
| | | Sequence |
| 24 | TTTTTTTTTTT | Termination |
| | | Sequence |
| 25 | TTTTTTTTTTTT | Termination |
| | | Sequence |
| 26 | AGCTCTGCTTATATAGACCTCCCACCGTACACGCCTACCG | CMV |
| | CCCATTTGCGTCAATGGGGCGGAGTTGTTACGACATTTTG | Promoter |
| | GAAAGTCCCGTTGATTTTGGTGCCAAAACAAACTCCCATT | |
| | GACGTCAATGGGGTGGAGACTTGGAAATCCCCGTGAGTC | |
| | AAACCGCTATCCACGCCCATTGATGTACTGCCAAAACCG | |
| | CATCAC | |
| 27 | CATGGTAATAGCGATGACTAATACGTAGATGTACTGCCA | CMV |
| | AGTAGGAAAGTCCCATAAGGTCATGTACTGGGCATAATG | Enhancer |
| | CCAGGCGGGCCATTTACCGTCATTGACGTCAATAGGGGG | |
| | CGTACTTGGCATATGATACACTTGATGTACTGCCAAGTGG | |
| | GCAGTTTACCGTAAATACTCCACCCATTGACGTCAATGGA | |
| | AAGTCCCTATTGGCGTTACTATGGGAACATACGTCATTAT | |
| | TGACGTCAATGGGGGGGGTCGTTGGGCGGTCAGCCAGG | |
| | CGGGCCATTTACCGTAAGTTATGTAACGCGGAACTCCATA | |
| | TATGGGCTATGAACTAATGACCCCGTAATTGATTACTATT | |
| | AATAACTAGTCAATAATCAATGTC | |
| 28 | TCAGCTGCCCTTGTACAGCTCGTCCATGCCGTGGGTGATG | GFP- |
| | CCAGCGGCGGTCACGAATTCCAGCAGCACCATGTGGTCC | 150TAG |
| | CGCTTCTCGTTGGGGTCCTTGCTCAGCACGCTCTGGGTGC | |
| | TCAGGTAGTGGTTGTCGGGCAGCAGCACGGGGCCATCTC | |
| | CGATGGGGGTGTTCTGCTGGTAGTGGTCGGCCAGCTGCA | |
| | CGCTGCCATCTTCCACGTTGTGCCGGATCTTGAAGTTGGC | |
| | CTTGATGCCGTTCTTCTGCTTGTCGGCGGTGATGTACACC | |
| | TAGTGGCTGTTGAAGTTGTACTCCAGCTTGTGGCCCAGGA | |
| | TGTTGCCGTCCTCTTTGAAATCGATGCCCTTCAGCTCGAT | |
| | CCGGTTCACGAGGGTGTCGCCCTCGAACTTCACTTCGGCT | |
| | CTGGTCTTGTAGGTGCCGTCGTCCTTGAAGCTGATGGTCC | |
| | GTTCCTGCACGTAGCCCTCGGGCATGGCGCTCTTGAAGAA | |
| | GTCGTGCCGCTTCATATGGTCGGGGTATCTGCTGAAGCAC | |
| | TGCACGCCGTAGGTCAGTGTGGTCACGAGGGTAGGCCAA | |
| | GGCACGGGCAGCTTGCCGGTGGTGCAGATGAACTCCAGG | |
| | GTCAGCTTGCCATTTGTGGCGTCGCCTTCGCCCTCTCCCC | |
| | GCACAGAGAACTTGTGGCCGTTCACGTCGCCATCCAGTTC | |
| | CACCAGGATGGGCACCACGCCGGTGAACAGTTCCTCGCC | |
| | CTTGGACACCAT | |
| 29 | CCATAGAGCCCACCGCATCCCCAGCATGCCTGCTATTGTC | BGH Poly A |
| | TTCCCAATCCTCCCCCTTGCTGTCCTGCCCCACCCCACCC | Signal |
| | CCCAGAATAGAATGACACCTACTCAGACAATGCGATGCA | |
| | ATTTCCTCATTTTATTAGGAAAGGACAGTGGGAGTGGCAC | |
| | CTTCCAGGGTCAAGGAAGGCACGGGGGAGGGGCAAACA | |
| | ACAGATGGCTGGCAACTAGAAGGCACAG | |
| 30 | CTGCAGTATTTAGCATGCCCCACCCATCTGCAAGGCATTC | Sup tRNA |
| | TGGATAGTGTCAAAACAGCCGGAAATCAAGTCCGTTTAT | V2.1 |
| | CTCAAACTTTAGCATTTTGGGAATAAATGATATTTGCTAT | expression |
| | GCTGGTTAAATTAGATTTTAGTTAAATTTCCTGCTGAAGC | cassette |
| | TCTAGTACGATAAGTAACTTGACCTAAGTGTAAAGTTGA | |
| | GATTTCCTTCAGGTTTATATAGCTTGTGCGCCGCCTGGGT | |
| | ACCTCGGGGGACGGTCCGGCGACCAGCGGGTCTCTAAAA | |
| | CCTAGCATAGCGGGGTTCGACACCCCGGTCTTCCATTTTT | |
| | TGCTAATCCGTATCGCTACCTGCAGTATTTAGCATGCCCC | |
| | ACCCATCTGCAAGGCATTCTGGATAGTGTCAAAACAGCC | |
| | GGAAATCAAGTCCGTTTATCTCAAACTTTAGCATTTTGGG | |
| | AATAAATGATATTTGCTATGCTGGTTAAATTAGATTTTAG | |
| | TTAAATTTCCTGCTGAAGCTCTAGTACGATAAGTAACTTG | |
| | ACCTAAGTGTAAAGTTGAGATTTCCTTCAGGTTTATATAG | |
| | CTTGTGCGCCGCCTGGGTACCTCGGGGGACGGTCCGGCG | |
| | ACCAGCGGGTCTCTAAAACCTAGCATAGCGGGGTTCGAC | |
| | ACCCCGGTCTTCCATTTTTTACCTGCATGGTACTCGTACTG | |
| | CAGTATTTAGCATGCCCCACCCATCTGCAAGGCATTCTGG | |
| | ATAGTGTCAAAACAGCCGGAAATCAAGTCCGTTTATCTC | |
| | AAACTTTAGCATTTTGGGAATAAATGATATTTGCTATGCT | |
| | GGTTAAATTAGATTTTAGTTAAATTTCCTGCTGAAGCTCT | |
| | AGTACGATAAGTAACTTGACCTAAGTGTAAAGTTGAGAT | |
| | TTCCTTCAGGTTTATATAGCTTGTGCGCCGCCTGGGTACC | |
| | TCGGGGGACGGTCCGGCGACCAGCGGGTCTCTAAAACCT | |
| | AGCATAGCGGGGTTCGACACCCCGGTCTTCCATTTTTT | |
| 31 | AGTGGGTTACATCGAACTGGATCTCAACAGCGGTAAGAT | Sup tRNA |
| | CCTTGAGAGTTTTCGCCCCGAAGAACGTTTTCCAATGATG | V2.1-GFP |
| | AGCACTTTTAAAGTTCTGCTATGTGGCGCGGTATTATCCC | plasmid |
| | GTATTGACGCCGGGCAAGAGCAACTCGGTCGCCGCATAC | |
| | ACTATTCTCAGAATGACTTGGTTGAGTACTCACCAGTCAC | |
| | AGAAAAGCATCTTACGGATGGCATGACAGTAAGAGAATT | |
| | ATGCAGTGCTGCCATAACCATGAGTGATAACACTGCGGC | |
| | CAACTTACTTCTGACAACGATCGGAGGACCGAAGGAGCT | |
| | AACCGCTTTTTTGCACAACATGGGGGATCATGTAACTCGC | |
| | CTTGATCGTTGGGAACCGGAGCTGAATGAAGCCATACCA | |
| | AACGACGAGCGTGACACCACGATGCCTGTAGCAATGGCA | |
| | ACAACGTTGCGCAAACTATTAACTGGCGAACTACTTACTC | |
| | TAGCTTCCCGGCAACAATTAATAGACTGGATGGAGGCGG | |
| | ATAAAGTTGCAGGACCACTTCTGCGCTCGGCCCTTCCGGC | |
| | TGGCTGGTTTATTGCTGATAAATCTGGAGCCGGTGAGCGT | |
| | GGGTCTCGCGGTATCATTGCAGCACTGGGGCCAGATGGT | |
| | AAGCCCTCCCGTATCGTAGTTATCTACACGACGGGGAGTC | |
| | AGGCAACTATGGATGAACGAAATAGACAGATCGCTGAGA | |
| | TAGGTGCCTCACTGATTAAGCATTGGTAACTGTCAGACCA | |
| | AGTTTACTCATATATACTTTAGATTGATTTAAAACTTCATT | |
| | TTTAATTTAAAAGGATCTAGGTGAAGATCCTTTTTGATAA | |
| | TCTCATGACCAAAATCCCTTAACGTGAGTTTTCGTTCCAC | |
| | TGAGCGTCAGACCCCGTAGAAAAGATCAAAGGATCTTCT | |
| | TGAGATCCTTTTTTTCTGCGCGTAATCTGCTGCTTGCAAA | |
| | CAAAAAAACCACCGCTACCAGCGGTGGTTTGTTTGCCGG | |
| | ATCAAGAGCTACCAACTCTTTTTCCGAAGGTAACTGGCTT | |
| | CAGCAGAGCGCAGATACCAAATACTGTTCTTCTAGTGTA | |
| | GCCGTAGTTAGGCCACCACTTCAAGAACTCTGTAGCACC | |
| | GCCTACATACCTCGCTCTGCTAATCCTGTTACCAGTGGCT | |
| | GCTGCCAGTGGCGATAAGTCGTGTCTTACCGGGTTGGACT | |
| | CAAGACGATAGTTACCGGATAAGGCGCAGCGGTCGGGCT | |
| | GAACGGGGGGTTCGTGCACACAGCCCAGCTTGGAGCGAA | |
| | CGACCTACACCGAACTGAGATACCTACAGCGTGAGCTAT | |
| | GAGAAAGCGCCACGCTTCCCGAAGGGAGAAAGGCGGAC | |
| | AGGTATCCGGTAAGCGGCAGGGTCGGAACAGGAGAGCG | |
| | CACGAGGGAGCTTCCAGGGGGAAACGCCTGGTATCTTTA | |
| | TAGTCCTGTCGGGTTTCGCCACCTCTGACTTGAGCGTCGA | |
| | TTTTTGTGATGCTCGTCAGGGGGGCGGAGCCTATGGAAA | |
| | AACGCCAGCAACGCGGCCTTTTTACGGTTCCTGGCCTTTT | |
| | GCTGGCCTTTTGCTCACATGTTCTTTCCTGCGTTATCCCCT | |
| | GATTCTGTGGATAACCGTATTACCGCCTTTGAGTGAGCTG | |
| | ATACCGCTCGCCGCAGCCGAACGACCGAGCGCAGCGAGT | |
| | CAGTGAGCGAGGAAGCGGAAGAGCGCCCAATACGCAAA | |
| | CCGCCTCTCCCCGCGCGTTGGCCGATTCATTAATGCAGCT | |
| | GGCACGACAGGTTTCCCGACTGGAAAGCGGGCAGTGAGC | |
| | GCAACGCAATTAATGTGAGTTAGCTCACTCATTAGGCACC | |
| | CCAGGCTTTACACTTTATGCTTCCGGCTCGTATGTTGTGT | |
| | GGAATTGTGAGCGGATAACAATTTCACACAGGAAACAGC | |
| | TATGACCATGATTACGCCAAGCTCTAGCTAGAGGTCGAC | |
| | GGTATACAGACATGATAAGATACATTGATGAGTTTGGAC | |
| | AAACCACAACTAGAATGCAGTGAAAAAAATGCTTTATTT | |
| | GTGAAATTTGTGATGCTATTGCTTTATTTGTAACCATTAT | |
| | AAGCTGCAATAAACAAGTTGGGGTGGGCGAAGAACTCCA | |
| | GCATGAGATCCCCGCGCTGGAGGATCATCCAGCCGGCGT | |
| | CCCGGAAAACGATTCCGAAGCCCAACCTTTCATAGAAGG | |
| | CGGCGGTGGAATCGAAATCTCGTGATGGCAGGTTGGTCG | |
| | TCGCTTGGTCGGTCATTTCGAACCCCAGAGTCCCGCTCAG | |
| | AAGAACTCGTCAAGAAGGCGATAGAAGGCGATGCGCTGC | |
| | GAATCGGGAGCGGCGATACCGTAAAGCACGAGGAAGCG | |
| | GTCAGCCCATTCGCCGCCAAGCTCTTCAGCAATATCACGG | |
| | GTAGCCAACGCTATGTCCTGATAGCGGTCCGCCACACCC | |
| | AGCCGGCCACAGTCGATGAATCCAGAAAAGCGGCCATTT | |
| | TCCACCATGATATTCGGCAAGCAGGCATCGCCATGGGTC | |
| | ACGACGAGATCCTCGCCGTCGGGCATGCGCGCCTTGAGC | |
| | CTGGCGAACAGTTCGGCTGGCGCGAGCCCCTGATGCTCTT | |
| | CGTCCAGATCATCCTGATCGACAAGACCGGCTTCCATCCG | |
| | AGTACGTGCTCGCTCGATGCGATGTTTCGCTTGGTGGTCG | |
| | AATGGGCAGGTAGCCGGATCAAGCGTATGCAGCCGCCGC | |
| | ATTGCATCAGCCATGATGGATACTTTCTCGGCAGGAGCA | |
| | AGGTGAGATGACAGGAGATCCTGCCCCGGCACTTCGCCC | |
| | AATAGCAGCCAGTCCCTTCCCGCTTCAGTGACAACGTCGA | |
| | GCACAGCTGCGCAAGGAACGCCCGTCGTGGCCAGCCACG | |
| | ATAGCCGCGCTGCCTCGTCCTGCAGTTCATTCAGGGCACC | |
| | GGACAGGTCGGTCTTGACAAAAAGAACCGGGCGCCCCTG | |
| | CGCTGACAGCCGGAACACGGCGGCATCAGAGCAGCCGAT | |
| | TGTCTGTTGTGCCCAGTCATAGCCGAATAGCCTCTCCACC | |
| | CAAGCGGCCGGAGAACCTGCGTGCAATCCATCTTGTTCA | |
| | ATCATGCGAAACGATCCTCATCCTGTCTCTTGATCAGATC | |
| | CGAAAATGGATATACAAGCTCCCGGGAGCTTTTTGCAAA | |
| | AGCCTAGGCCTCCAAAAAAGCCTCCTCACTACTTCTGGAA | |
| | TAGCTCAGAGGCAGAGGCGGCCTCGGCCTCTGCATAAAT | |
| | AAAAAAAATTAGTCAGCCATGGGGCGGAGAATGGGCGG | |
| | AACTGGGCGGAGTTAGGGGCGGGATGGGCGGAGTTAGG | |
| | GGCGGGACTATGGTTGCTGACTAATTGAGATGCATGCTTT | |
| | GCATACTTCTGCCTGCTGGGGAGCCTGGGGACTTTCCACA | |
| | CCTGGTTGCTGACTAATTGAGATGCATGCTTTGCATACTT | |
| | CTGCCTGCTGGGGAGCCTGGGGACTTTCCACACCCTAACT | |
| | GACACACATTCCACAGAATTAATTCGCGTTAAATTTTTGT | |
| | TAAATCAGCTCATTTTTTAACCAATAGGCCGAAATCGGCA | |
| | AAATCCCTTATAAATCAAAAGAATAGACCGAGATAGGGT | |
| | TGAGTGTTGTTCCAGTTTGGAACAAGAGTCCACTATTAAA | |
| | GAACGTGGACTCCAACGTCAAAGGGCGAAAAACCGTCTA | |
| | TCAGGGCGATGGCCCACTACGTGAACCATCACCCTAATC | |
| | AAGTTTTTTGGGGTCGAGGTGCCGTAAAGCACTAAATCG | |
| | GAACCCTAAAGGGAGCCCCCCGATTTAGAGCTTGACGGG | |
| | GAAAGCCGGCGAACGTGGCGAGAAAGGAAGGGAAGAAA | |
| | GCGAAAGGAGCGGGCGCTAGGGCGCTGGCAAGTGTAGC | |
| | GGTCACGCTGCGCGTAACCACCACACCCGCCGCGCTTAA | |
| | TGCGCCGCTACAGGGCGCGTGGGGATACCCCCTAGAGCC | |
| | CCAGCTGGTTCTTTCCGCCTCAGAAGCCATAGAGCCCACC | |
| | GCATCCCCAGCATGCCTGCTATTGTCTTCCCAATCCTCCC | |
| | CCTTGCTGTCCTGCCCCACCCCACCCCCCAGAATAGAATG | |
| | ACACCTACTCAGACAATGCGATGCAATTTCCTCATTTTAT | |
| | TAGGAAAGGACAGTGGGAGTGGCACCTTCCAGGGTCAAG | |
| | GAAGGCACGGGGGAGGGGCAAACAACAGATGGCTGGCA | |
| | ACTAGAAGGCACAGTCGAGGCTGATCAGCGGGTTTAAAC | |
| | GGGCCCTCTAGACTCGAGTCATCAGCTGCCCTTGTACAGC | |
| | TCGTCCATGCCGTGGGTGATGCCAGCGGCGGTCACGAAT | |
| | TCCAGCAGCACCATGTGGTCCCGCTTCTCGTTGGGGTCCT | |
| | TGCTCAGCACGCTCTGGGTGCTCAGGTAGTGGTTGTCGGG | |
| | CAGCAGCACGGGGCCATCTCCGATGGGGGTGTTCTGCTG | |
| | GTAGTGGTCGGCCAGCTGCACGCTGCCATCTTCCACGTTG | |
| | TGCCGGATCTTGAAGTTGGCCTTGATGCCGTTCTTCTGCT | |
| | TGTCGGCGGTGATGTACACCTAGTGGCTGTTGAAGTTGTA | |
| | CTCCAGCTTGTGGCCCAGGATGTTGCCGTCCTCTTTGAAA | |
| | TCGATGCCCTTCAGCTCGATCCGGTTCACGAGGGTGTCGC | |
| | CCTCGAACTTCACTTCGGCTCTGGTCTTGTAGGTGCCGTC | |
| | GTCCTTGAAGCTGATGGTCCGTTCCTGCACGTAGCCCTCG | |
| | GGCATGGCGCTCTTGAAGAAGTCGTGCCGCTTCATATGGT | |
| | CGGGGTATCTGCTGAAGCACTGCACGCCGTAGGTCAGTG | |
| | TGGTCACGAGGGTAGGCCAAGGCACGGGCAGCTTGCCGG | |
| | TGGTGCAGATGAACTCCAGGGTCAGCTTGCCATTTGTGGC | |
| | GTCGCCTTCGCCCTCTCCCCGCACAGAGAACTTGTGGCCG | |
| | TTCACGTCGCCATCCAGTTCCACCAGGATGGGCACCACGC | |
| | CGGTGAACAGTTCCTCGCCCTTGGACACCATGGTGGCGA | |
| | ATTCCACCACACTGGACTAGTGGATCCGAGCTCGGTACC | |
| | AAGCTTAAGTTTAAACGCTAGCCAGCTTGGGTCTCCCTAT | |
| | AGTGAGTCGTATTAATTTCGATAAGCCAGTAAGCAGTGG | |
| | GTTCTCTAGTTAGCCAGAGAGCTCTGCTTATATAGACCTC | |
| | CCACCGTACACGCCTACCGCCCATTTGCGTCAATGGGGCG | |
| | GAGTTGTTACGACATTTTGGAAAGTCCCGTTGATTTTGGT | |
| | GCCAAAACAAACTCCCATTGACGTCAATGGGGTGGAGAC | |
| | TTGGAAATCCCCGTGAGTCAAACCGCTATCCACGCCCATT | |
| | GATGTACTGCCAAAACCGCATCACCATGGTAATAGCGAT | |
| | GACTAATACGTAGATGTACTGCCAAGTAGGAAAGTCCCA | |
| | TAAGGTCATGTACTGGGCATAATGCCAGGCGGGCCATTT | |
| | ACCGTCATTGACGTCAATAGGGGGCGTACTTGGCATATG | |
| | ATACACTTGATGTACTGCCAAGTGGGCAGTTTACCGTAAA | |
| | TACTCCACCCATTGACGTCAATGGAAAGTCCCTATTGGCG | |
| | TTACTATGGGAACATACGTCATTATTGACGTCAATGGGCG | |
| | GGGGTCGTTGGGCGGTCAGCCAGGCGGGCCATTTACCGT | |
| | AAGTTATGTAACGCGGAACTCCATATATGGGCTATGAAC | |
| | TAATGACCCCGTAATTGATTACTATTAATAACTAGTCAAT | |
| | AATCAATGTCAACGCGTATATCTGGCCCGTACATCGCGA | |
| | AGCAGCGCAAAACGCCTAACCCTAAGCAGATTCTTCATG | |
| | CAATTGTCGGTCAAGCCTTGCCTTGTTGTAGCTTAAATTT | |
| | TGCTCGCGCACTACTCAGCGACCTCCTCGCAGATCCTTTC | |
| | TAGCATTTGACAGGCACATTATGCTGGCGCCTGCAGTATT | |
| | TAGCATGCCCCACCCATCTGCAAGGCATTCTGGATAGTGT | |
| | CAAAACAGCCGGAAATCAAGTCCGTTTATCTCAAACTTTA | |
| | GCATTTTGGGAATAAATGATATTTGCTATGCTGGTTAAAT | |
| | TAGATTTTAGTTAAATTTCCTGCTGAAGCTCTAGTACGAT | |
| | AAGTAACTTGACCTAAGTGTAAAGTTGAGATTTCCTTCAG | |
| | GTTTATATAGCTTGTGCGCCGCCTGGGTACCTCGGGGGAC | |
| | GGTCCGGCGACCAGCGGGTCTCTAAAACCTAGCATAGCG | |
| | GGGTTCGACACCCCGGTCTTCCATTTTTTGCTAATCCGTA | |
| | TCGCTACCTGCAGTATTTAGCATGCCCCACCCATCTGCAA | |
| | GGCATTCTGGATAGTGTCAAAACAGCCGGAAATCAAGTC | |
| | CGTTTATCTCAAACTTTAGCATTTTGGGAATAAATGATAT | |
| | TTGCTATGCTGGTTAAATTAGATTTTAGTTAAATTTCCTGC | |
| | TGAAGCTCTAGTACGATAAGTAACTTGACCTAAGTGTAA | |
| | AGTTGAGATTTCCTTCAGGTTTATATAGCTTGTGCGCCGC | |
| | CTGGGTACCTCGGGGGACGGTCCGGCGACCAGCGGGTCT | |
| | CTAAAACCTAGCATAGCGGGGTTCGACACCCCGGTCTTCC | |
| | ATTTTTTACCTGCATGGTACTCGTACTGCAGTATTTAGCA | |
| | TGCCCCACCCATCTGCAAGGCATTCTGGATAGTGTCAAAA | |
| | CAGCCGGAAATCAAGTCCGTTTATCTCAAACTTTAGCATT | |
| | TTGGGAATAAATGATATTTGCTATGCTGGTTAAATTAGAT | |
| | TTTAGTTAAATTTCCTGCTGAAGCTCTAGTACGATAAGTA | |
| | ACTTGACCTAAGTGTAAAGTTGAGATTTCCTTCAGGTTTA | |
| | TATAGCTTGTGCGCCGCCTGGGTACCTCGGGGGACGGTCC | |
| | GGCGACCAGCGGGTCTCTAAAACCTAGCATAGCGGGGTT | |
| | CGACACCCCGGTCTTCCATTTTTTGGATCCTCTAGAGTCG | |
| | ACCTGCAGATCCTTAGACTAGTTGGCCGTGGAGGCCTTTT | |
| | CCCCGTATCCCCCCAGGTGTCTGCAGGCTCAAAGAGCAG | |
| | CGAGAAGCGTTCAGAGGAAAGCGATCCCGTGCCACCTTC | |
| | CCCGTGCCCGGGCTGTCCCCGCACGCTGCCGGCTCGGGG | |
| | ATGCGGGGGGAGCGCCGGACCGGAGCGGAGCCCCGGGC | |
| | GGCTCGCTGCTGCCCCCTAGCGGGGGAGGGACGTAATTA | |
| | CATCCCTGGGGGCTTTGGGGGGGGGCTGTCCCTCATAAA | |
| | AGTTTTGTTACTTTATAGAAGAAATTTTGAGTTTTTGTTTT | |
| | TTTTAATAAATAAATAAACATAAATAAATTGTTTGTTGAA | |
| | TTTATTATTAGTATGTAAGTGTAAATATAATAAAACTTAA | |
| | TATCTATTCAAATTAATAAATAAACCTCGATATACAGACC | |
| | GATAAAACACATGCGTCAATTTTACACATGATTATCTTTA | |
| | ACGTACGTCACAATATGATTATCTTTCTAGGGTTAATCTA | |
| | GCTGCGTGTTCTGCAGCGTGTCGAGCATCTTCATCTGCTC | |
| | CATCACGCTGTAAAACACATTTGCACCGCGAGTCTGCCCG | |
| | TCCTCCACGGGTTCAAAAACGTGAATGAACGAGGCGCGC | |
| | TCACTGGCCGTCGTTTTACAACACACAAGCAGGGAGCAG | |
| | ATACTGGCTTAACTATGCGGCATCAGAGCAGATTGTACTG | |
| | AGAGTGCACCATAGGGGATCGGGAGATCTCCCGATCCGT | |
| | CGACGTCAGGTGGCACTTTTCGGGGAAATGTGCGCGGAA | |
| | CCCCTATTTGTTTATTTTTCTAAATACATTCAAATATGTAT | |
| | CCGCTCATGAGACAATAACCCTGATAAATGCTTCAATAAT | |
| | ATTGAAAAAGGAAGAGTATGAGTATTCAACATTTCCGTG | |
| | TCGCCCTTATTCCCTTTTTTGCGGCATTTTGCCTTCCTGTT | |
| | TTTGCTCACCCAGAAACGCTGGTGAAAGTAAAAGATGCT | |
| | GAAGATCAGTTGGGTGCACG | |
| 32 | TCAACCTGGGTGCGCTGCGTCTGTGGTTCCTGCGGGCGGT | SEAP40TAG |
| | GCAAGGTCACAGGCGGTGTATGGTTCGAGGCACGCTGCG | |
| | AAAGCCATTACATGGGCTATGAAAGTCTGTTCTTGCACAC | |
| | CATGCACAAGGTGTGCCTGGGGGCCCTGAGCGAAAACAG | |
| | CGACATCTTCGCCAGCATGCGTTTCCTCATCCAGTGGGAC | |
| | TGCGCTTTGCTGCCGGTATTCTGGTGAGCCTGACTCTGAT | |
| | TCAGTTACATCTGGTCTAGCTCCGTCCTTGAGTACGTAGC | |
| | CCGGTCCGTTTCCATACAACAACACCGTGTAGGCTTTCCG | |
| | ATCGCGCGCTTTTCCCGGTGCAAGCCCGAAAATACTACTG | |
| | CCTCGCAACGGGTACCCTCCAAATGAGAAAACATGGCTG | |
| | TGATCTGCGGTTACGAGAGAAAGTGTGTCCTCTTCAGATG | |
| | TCAATTGTCCCGCTCGCTCTATTGCGTCATCAAACATAAT | |
| | TGTCTCTGTCAGCGCGCGATAGGCCCGAGACTCATGATGT | |
| | CCATGATCTATCCTACCGCCCTCAACGAAAAGGAAGAAC | |
| | CCTCTAGGGTTTCTGGAAAGAAGTCGCAAAGCAGCCTCC | |
| | GTCATCTCCATAAGAGAAGGGTCAAGCGTACTGTCGCGG | |
| | TGTATCTCGTACTTCATGTCGCCAGGTTCGAAAAGCCCCA | |
| | TCAGATGGGTGACTGACGGATCCAGGCTAGCTTGCATCA | |
| | ATTCAGTTCTATTCCATACATATCGCGCACCTTGTCTCTTA | |
| | GCCAGCCATTCTTGGACCAGGTTCTTACCATCCAAGCGGG | |
| | TGCCTCCTTGGGAGTAGTCATCCGGGTATTCTGGGTCAGG | |
| | AGTCCCCATTCGGAACATGTACTTTCTCCCACCACCCAGG | |
| | ATGACATCTATATCCATATTGGAGATCAACTGCGTCGCGA | |
| | TGTCCTGGCACCCTTCTTGCCTTGCGGAGGCCGGGACATC | |
| | TGCGTCAGAATACCAGTTTCTGTTCACGGTATGGGCATAC | |
| | GTCCCGGCCGGGGACGCGTGTTGCACCCTAGTAGTCGTC | |
| | ACCACCCCGACACTTTTACCCGCTTTCTTTGCCCGGTTCAT | |
| | GACGCTAATAACCTCGTTACCGCGTGTGGTATTACATTGG | |
| | TTAAACCGAGCGGCAGCAGAAAGTCCGATAGTTTGAAAG | |
| | TTTCCTTTCACTCCACAGAGGTACGCAGTTGCAGTTGCAC | |
| | CGGAATCAGGTACGTGTTTATCCACGTTGTAGGTTTTGCT | |
| | AAGGGCCACATAGGGGAAGCGGTCCATTGCAAGCGGTAT | |
| | TTCAGGTCCCAGTTTGTCTTTTTTCTGACCTTTCAGTATTC | |
| | TTGCTGCTGTGACGGTGGAGACGCCCATGCCATCTCCCAG | |
| | AAAAATAATAAGGTTTTTCGCCGCTGTTTGTGCTGGCTGC | |
| | AGCTTCTACGCAGCTCCGAGAGCCTCTGCTGCTTCTCTAT | |
| | TCCAAAAATCGGGATTTTCTTCTTCTACGGGTATTATCCC | |
| | CAGGGACAGCTGAAGTCGCAGGCCAAGCAACAAGAGGA | |
| | GAAGCAACAT | |
| 33 | ACCAAACGACGAGCGTGACACCACGATGCCTGTAGCAAT | Sup tRNA |
| | GGCAACAACGTTGCGCAAACTATTAACTGGCGAACTACT | V2.1-SEAP |
| | TACTCTAGCTTCCCGGCAACAATTAATAGACTGGATGGA | |
| | GGCGGATAAAGTTGCAGGACCACTTCTGCGCTCGGCCCTT | |
| | CCGGCTGGCTGGTTTATTGCTGATAAATCTGGAGCCGGTG | |
| | AGCGTGGGTCTCGCGGTATCATTGCAGCACTGGGGCCAG | |
| | ATGGTAAGCCCTCCCGTATCGTAGTTATCTACACGACGGG | |
| | GAGTCAGGCAACTATGGATGAACGAAATAGACAGATCGC | |
| | TGAGATAGGTGCCTCACTGATTAAGCATTGGTAACTGTCA | |
| | GACCAAGTTTACTCATATATACTTTAGATTGATTTAAAAC | |
| | TTCATTTTTAATTTAAAAGGATCTAGGTGAAGATCCTTTT | |
| | TGATAATCTCATGACCAAAATCCCTTAACGTGAGTTTTCG | |
| | TTCCACTGAGCGTCAGACCCCGTAGAAAAGATCAAAGGA | |
| | TCTTCTTGAGATCCTTTTTTTCTGCGCGTAATCTGCTGCTT | |
| | GCAAACAAAAAAACCACCGCTACCAGCGGTGGTTTGTTT | |
| | GCCGGATCAAGAGCTACCAACTCTTTTTCCGAAGGTAACT | |
| | GGCTTCAGCAGAGCGCAGATACCAAATACTGTTCTTCTAG | |
| | TGTAGCCGTAGTTAGGCCACCACTTCAAGAACTCTGTAGC | |
| | ACCGCCTACATACCTCGCTCTGCTAATCCTGTTACCAGTG | |
| | GCTGCTGCCAGTGGCGATAAGTCGTGTCTTACCGGGTTGG | |
| | ACTCAAGACGATAGTTACCGGATAAGGCGCAGCGGTCGG | |
| | GCTGAACGGGGGGTTCGTGCACACAGCCCAGCTTGGAGC | |
| | GAACGACCTACACCGAACTGAGATACCTACAGCGTGAGC | |
| | TATGAGAAAGCGCCACGCTTCCCGAAGGGAGAAAGGCGG | |
| | ACAGGTATCCGGTAAGCGGCAGGGTCGGAACAGGAGAG | |
| | CGCACGAGGGAGCTTCCAGGGGGAAACGCCTGGTATCTT | |
| | TATAGTCCTGTCGGGTTTCGCCACCTCTGACTTGAGCGTC | |
| | GATTTTTGTGATGCTCGTCAGGGGGGCGGAGCCTATGGA | |
| | AAAACGCCAGCAACGCGGCCTTTTTACGGTTCCTGGCCTT | |
| | TTGCTGGCCTTTTGCTCACATGTTCTTTCCTGCGTTATCCC | |
| | CTGATTCTGTGGATAACCGTATTACCGCCTTTGAGTGAGC | |
| | TGATACCGCTCGCCGCAGCCGAACGACCGAGCGCAGCGA | |
| | GTCAGTGAGCGAGGAAGCGGAAGAGCGCCCAATACGCA | |
| | AACCGCCTCTCCCCGCGCGTTGGCCGATTCATTAATGCAG | |
| | CTGGCACGACAGGTTTCCCGACTGGAAAGCGGGCAGTGA | |
| | GCGCAACGCAATTAATGTGAGTTAGCTCACTCATTAGGC | |
| | ACCCCAGGCTTTACACTTTATGCTTCCGGCTCGTATGTTG | |
| | TGTGGAATTGTGAGCGGATAACAATTTCACACAGGAAAC | |
| | AGCTATGACCATGATTACGCCAAGCTCTAGCTAGAGGTC | |
| | GACGGTATACAGACATGATAAGATACATTGATGAGTTTG | |
| | GACAAACCACAACTAGAATGCAGTGAAAAAAATGCTTTA | |
| | TTTGTGAAATTTGTGATGCTATTGCTTTATTTGTAACCATT | |
| | ATAAGCTGCAATAAACAAGTTGGGGTGGGCGAAGAACTC | |
| | CAGCATGAGATCCCCGCGCTGGAGGATCATCCAGCCGGC | |
| | GTCCCGGAAAACGATTCCGAAGCCCAACCTTTCATAGAA | |
| | GGCGGCGGTGGAATCGAAATCTCGTGATGGCAGGTTGGT | |
| | CGTCGCTTGGTCGGTCATTTCGAACCCCAGAGTCCCGCTC | |
| | AGAAGAACTCGTCAAGAAGGCGATAGAAGGCGATGCGCT | |
| | GCGAATCGGGAGCGGCGATACCGTAAAGCACGAGGAAG | |
| | CGGTCAGCCCATTCGCCGCCAAGCTCTTCAGCAATATCAC | |
| | GGGTAGCCAACGCTATGTCCTGATAGCGGTCCGCCACAC | |
| | CCAGCCGGCCACAGTCGATGAATCCAGAAAAGCGGCCAT | |
| | TTTCCACCATGATATTCGGCAAGCAGGCATCGCCATGGGT | |
| | CACGACGAGATCCTCGCCGTCGGGCATGCGCGCCTTGAG | |
| | CCTGGCGAACAGTTCGGCTGGCGCGAGCCCCTGATGCTCT | |
| | TCGTCCAGATCATCCTGATCGACAAGACCGGCTTCCATCC | |
| | GAGTACGTGCTCGCTCGATGCGATGTTTCGCTTGGTGGTC | |
| | GAATGGGCAGGTAGCCGGATCAAGCGTATGCAGCCGCCG | |
| | CATTGCATCAGCCATGATGGATACTTTCTCGGCAGGAGCA | |
| | AGGTGAGATGACAGGAGATCCTGCCCCGGCACTTCGCCC | |
| | AATAGCAGCCAGTCCCTTCCCGCTTCAGTGACAACGTCGA | |
| | GCACAGCTGCGCAAGGAACGCCCGTCGTGGCCAGCCACG | |
| | ATAGCCGCGCTGCCTCGTCCTGCAGTTCATTCAGGGCACC | |
| | GGACAGGTCGGTCTTGACAAAAAGAACCGGGCGCCCCTG | |
| | CGCTGACAGCCGGAACACGGCGGCATCAGAGCAGCCGAT | |
| | TGTCTGTTGTGCCCAGTCATAGCCGAATAGCCTCTCCACC | |
| | CAAGCGGCCGGAGAACCTGCGTGCAATCCATCTTGTTCA | |
| | ATCATGCGAAACGATCCTCATCCTGTCTCTTGATCAGATC | |
| | CGAAAATGGATATACAAGCTCCCGGGAGCTTTTTGCAAA | |
| | AGCCTAGGCCTCCAAAAAAGCCTCCTCACTACTTCTGGAA | |
| | TAGCTCAGAGGCAGAGGCGGCCTCGGCCTCTGCATAAAT | |
| | AAAAAAAATTAGTCAGCCATGGGGCGGAGAATGGGCGG | |
| | AACTGGGCGGAGTTAGGGGGGGGATGGGCGGAGTTAGG | |
| | GGCGGGACTATGGTTGCTGACTAATTGAGATGCATGCTTT | |
| | GCATACTTCTGCCTGCTGGGGAGCCTGGGGACTTTCCACA | |
| | CCTGGTTGCTGACTAATTGAGATGCATGCTTTGCATACTT | |
| | CTGCCTGCTGGGGAGCCTGGGGACTTTCCACACCCTAACT | |
| | GACACACATTCCACAGAATTAATTCGCGTTAAATTTTTGT | |
| | TAAATCAGCTCATTTTTTAACCAATAGGCCGAAATCGGCA | |
| | AAATCCCTTATAAATCAAAAGAATAGACCGAGATAGGGT | |
| | TGAGTGTTGTTCCAGTTTGGAACAAGAGTCCACTATTAAA | |
| | GAACGTGGACTCCAACGTCAAAGGGCGAAAAACCGTCTA | |
| | TCAGGGCGATGGCCCACTACGTGAACCATCACCCTAATC | |
| | AAGTTTTTTGGGGTCGAGGTGCCGTAAAGCACTAAATCG | |
| | GAACCCTAAAGGGAGCCCCCCGATTTAGAGCTTGACGGG | |
| | GAAAGCCGGCGAACGTGGCGAGAAAGGAAGGGAAGAAA | |
| | GCGAAAGGAGCGGGCGCTAGGGCGCTGGCAAGTGTAGC | |
| | GGTCACGCTGCGCGTAACCACCACACCCGCCGCGCTTAA | |
| | TGCGCCGCTACAGGGCGCGTGGGGATACCCCCTAGAGCC | |
| | CCAGCTGGTTCTTTCCGCCTCAGAAGCCATAGAGCCCACC | |
| | GCATCCCCAGCATGCCTGCTATTGTCTTCCCAATCCTCCC | |
| | CCTTGCTGTCCTGCCCCACCCCACCCCCCAGAATAGAATG | |
| | ACACCTACTCAGACAATGCGATGCAATTTCCTCATTTTAT | |
| | TAGGAAAGGACAGTGGGAGTGGCACCTTCCAGGGTCAAG | |
| | GAAGGCACGGGGGAGGGGCAAACAACAGATGGCTGGCA | |
| | ACTAGAAGGCACAGTCGAGGCTGATCAGCGGGTTTAAAC | |
| | GGGCCCTCTAGACTCGAGTCAACCTGGGTGCGCTGCGTCT | |
| | GTGGTTCCTGCGGGCGGTGCAAGGTCACAGGCGGTGTAT | |
| | GGTTCGAGGCACGCTGCGAAAGCCATTACATGGGCTATG | |
| | AAAGTCTGTTCTTGCACACCATGCACAAGGTGTGCCTGGG | |
| | GGCCCTGAGCGAAAACAGCGACATCTTCGCCAGCATGCG | |
| | TTTCCTCATCCAGTGGGACTGCGCTTTGCTGCCGGTATTC | |
| | TGGTGAGCCTGACTCTGATTCAGTTACATCTGGTCTAGCT | |
| | CCGTCCTTGAGTACGTAGCCCGGTCCGTTTCCATACAACA | |
| | ACACCGTGTAGGCTTTCCGATCGCGCGCTTTTCCCGGTGC | |
| | AAGCCCGAAAATACTACTGCCTCGCAACGGGTACCCTCC | |
| | AAATGAGAAAACATGGCTGTGATCTGCGGTTACGAGAGA | |
| | AAGTGTGTCCTCTTCAGATGTCAATTGTCCCGCTCGCTCT | |
| | ATTGCGTCATCAAACATAATTGTCTCTGTCAGCGCGCGAT | |
| | AGGCCCGAGACTCATGATGTCCATGATCTATCCTACCGCC | |
| | CTCAACGAAAAGGAAGAACCCTCTAGGGTTTCTGGAAAG | |
| | AAGTCGCAAAGCAGCCTCCGTCATCTCCATAAGAGAAGG | |
| | GTCAAGCGTACTGTCGCGGTGTATCTCGTACTTCATGTCG | |
| | CCAGGTTCGAAAAGCCCCATCAGATGGGTGACTGACGGA | |
| | TCCAGGCTAGCTTGCATCAATTCAGTTCTATTCCATACAT | |
| | ATCGCGCACCTTGTCTCTTAGCCAGCCATTCTTGGACCAG | |
| | GTTCTTACCATCCAAGCGGGTGCCTCCTTGGGAGTAGTCA | |
| | TCCGGGTATTCTGGGTCAGGAGTCCCCATTCGGAACATGT | |
| | ACTTTCTCCCACCACCCAGGATGACATCTATATCCATATT | |
| | GGAGATCAACTGCGTCGCGATGTCCTGGCACCCTTCTTGC | |
| | CTTGCGGAGGCCGGGACATCTGCGTCAGAATACCAGTTT | |
| | CTGTTCACGGTATGGGCATACGTCCCGGCCGGGGACGCG | |
| | TGTTGCACCCTAGTAGTCGTCACCACCCCGACACTTTTAC | |
| | CCGCTTTCTTTGCCCGGTTCATGACGCTAATAACCTCGTT | |
| | ACCGCGTGTGGTATTACATTGGTTAAACCGAGCGGCAGC | |
| | AGAAAGTCCGATAGTTTGAAAGTTTCCTTTCACTCCACAG | |
| | AGGTACGCAGTTGCAGTTGCACCGGAATCAGGTACGTGT | |
| | TTATCCACGTTGTAGGTTTTGCTAAGGGCCACATAGGGGA | |
| | AGCGGTCCATTGCAAGCGGTATTTCAGGTCCCAGTTTGTC | |
| | TTTTTTCTGACCTTTCAGTATTCTTGCTGCTGTGACGGTGG | |
| | AGACGCCCATGCCATCTCCCAGAAAAATAATAAGGTTTTT | |
| | CGCCGCTGTTTGTGCTGGCTGCAGCTTCTACGCAGCTCCG | |
| | AGAGCCTCTGCTGCTTCTCTATTCCAAAAATCGGGATTTT | |
| | CTTCTTCTACGGGTATTATCCCCAGGGACAGCTGAAGTCG | |
| | CAGGCCAAGCAACAAGAGGAGAAGCAACATGGTGGCGA | |
| | ATTCCACCACACTGGACTAGTGGATCCGAGCTCGGTACC | |
| | AAGCTTAAGTTTAAACGCTAGCCAGCTTGGGTCTCCCTAT | |
| | AGTGAGTCGTATTAATTTCGATAAGCCAGTAAGCAGTGG | |
| | GTTCTCTAGTTAGCCAGAGAGCTCTGCTTATATAGACCTC | |
| | CCACCGTACACGCCTACCGCCCATTTGCGTCAATGGGGCG | |
| | GAGTTGTTACGACATTTTGGAAAGTCCCGTTGATTTTGGT | |
| | GCCAAAACAAACTCCCATTGACGTCAATGGGGTGGAGAC | |
| | TTGGAAATCCCCGTGAGTCAAACCGCTATCCACGCCCATT | |
| | GATGTACTGCCAAAACCGCATCACCATGGTAATAGCGAT | |
| | GACTAATACGTAGATGTACTGCCAAGTAGGAAAGTCCCA | |
| | TAAGGTCATGTACTGGGCATAATGCCAGGCGGGCCATTT | |
| | ACCGTCATTGACGTCAATAGGGGGCGTACTTGGCATATG | |
| | ATACACTTGATGTACTGCCAAGTGGGCAGTTTACCGTAAA | |
| | TACTCCACCCATTGACGTCAATGGAAAGTCCCTATTGGCG | |
| | TTACTATGGGAACATACGTCATTATTGACGTCAATGGGCG | |
| | GGGGTCGTTGGGCGGTCAGCCAGGCGGGCCATTTACCGT | |
| | AAGTTATGTAACGCGGAACTCCATATATGGGCTATGAAC | |
| | TAATGACCCCGTAATTGATTACTATTAATAACTAGTCAAT | |
| | AATCAATGTCAACGCGTATATCTGGCCCGTACATCGCGA | |
| | AGCAGCGCAAAACGCCTAACCCTAAGCAGATTCTTCATG | |
| | CAATTGTCGGTCAAGCCTTGCCTTGTTGTAGCTTAAATTT | |
| | TGCTCGCGCACTACTCAGCGACCTCCTCGCAGATCCTTTC | |
| | TAGCATTTGACAGGCACATTATGCTGGCGCCTGCAGTATT | |
| | TAGCATGCCCCACCCATCTGCAAGGCATTCTGGATAGTGT | |
| | CAAAACAGCCGGAAATCAAGTCCGTTTATCTCAAACTTTA | |
| | GCATTTTGGGAATAAATGATATTTGCTATGCTGGTTAAAT | |
| | TAGATTTTAGTTAAATTTCCTGCTGAAGCTCTAGTACGAT | |
| | AAGTAACTTGACCTAAGTGTAAAGTTGAGATTTCCTTCAG | |
| | GTTTATATAGCTTGTGCGCCGCCTGGGTACCTCGGGGGAC | |
| | GGTCCGGCGACCAGCGGGTCTCTAAAACCTAGCATAGCG | |
| | GGGTTCGACACCCCGGTCTTCCATTTTTTGCTAATCCGTA | |
| | TCGCTACCTGCAGTATTTAGCATGCCCCACCCATCTGCAA | |
| | GGCATTCTGGATAGTGTCAAAACAGCCGGAAATCAAGTC | |
| | CGTTTATCTCAAACTTTAGCATTTTGGGAATAAATGATAT | |
| | TTGCTATGCTGGTTAAATTAGATTTTAGTTAAATTTCCTGC | |
| | TGAAGCTCTAGTACGATAAGTAACTTGACCTAAGTGTAA | |
| | AGTTGAGATTTCCTTCAGGTTTATATAGCTTGTGCGCCGC | |
| | CTGGGTACCTCGGGGGACGGTCCGGCGACCAGCGGGTCT | |
| | CTAAAACCTAGCATAGCGGGGTTCGACACCCCGGTCTTCC | |
| | ATTTTTTACCTGCATGGTACTCGTACTGCAGTATTTAGCA | |
| | TGCCCCACCCATCTGCAAGGCATTCTGGATAGTGTCAAAA | |
| | CAGCCGGAAATCAAGTCCGTTTATCTCAAACTTTAGCATT | |
| | TTGGGAATAAATGATATTTGCTATGCTGGTTAAATTAGAT | |
| | TTTAGTTAAATTTCCTGCTGAAGCTCTAGTACGATAAGTA | |
| | ACTTGACCTAAGTGTAAAGTTGAGATTTCCTTCAGGTTTA | |
| | TATAGCTTGTGCGCCGCCTGGGTACCTCGGGGGACGGTCC | |
| | GGCGACCAGCGGGTCTCTAAAACCTAGCATAGCGGGGTT | |
| | CGACACCCCGGTCTTCCATTTTTTGGATCCTCTAGAGTCG | |
| | ACCTGCAGATCCTTAGACTAGTTGGCCGTGGAGGCCTTTT | |
| | CCCCGTATCCCCCCAGGTGTCTGCAGGCTCAAAGAGCAG | |
| | CGAGAAGCGTTCAGAGGAAAGCGATCCCGTGCCACCTTC | |
| | CCCGTGCCCGGGCTGTCCCCGCACGCTGCCGGCTCGGGG | |
| | ATGCGGGGGGAGCGCCGGACCGGAGCGGAGCCCCGGGC | |
| | GGCTCGCTGCTGCCCCCTAGCGGGGGAGGGACGTAATTA | |
| | CATCCCTGGGGGCTTTGGGGGGGGGCTGTCCCTCATAAA | |
| | AGTTTTGTTACTTTATAGAAGAAATTTTGAGTTTTTGTTTT | |
| | TTTTAATAAATAAATAAACATAAATAAATTGTTTGTTGAA | |
| | TTTATTATTAGTATGTAAGTGTAAATATAATAAAACTTAA | |
| | TATCTATTCAAATTAATAAATAAACCTCGATATACAGACC | |
| | GATAAAACACATGCGTCAATTTTACACATGATTATCTTTA | |
| | ACGTACGTCACAATATGATTATCTTTCTAGGGTTAATCTA | |
| | GCTGCGTGTTCTGCAGCGTGTCGAGCATCTTCATCTGCTC | |
| | CATCACGCTGTAAAACACATTTGCACCGCGAGTCTGCCCG | |
| | TCCTCCACGGGTTCAAAAACGTGAATGAACGAGGCGCGC | |
| | TCACTGGCCGTCGTTTTACAACACACAAGCAGGGAGCAG | |
| | ATACTGGCTTAACTATGCGGCATCAGAGCAGATTGTACTG | |
| | AGAGTGCACCATAGGGGATCGGGAGATCTCCCGATCCGT | |
| | CGACGTCAGGTGGCACTTTTCGGGGAAATGTGCGCGGAA | |
| | CCCCTATTTGTTTATTTTTCTAAATACATTCAAATATGTAT | |
| | CCGCTCATGAGACAATAACCCTGATAAATGCTTCAATAAT | |
| | ATTGAAAAAGGAAGAGTATGAGTATTCAACATTTCCGTG | |
| | TCGCCCTTATTCCCTTTTTTGCGGCATTTTGCCTTCCTGTT | |
| | TTTGCTCACCCAGAAACGCTGGTGAAAGTAAAAGATGCT | |
| | GAAGATCAGTTGGGTGCACGAGTGGGTTACATCGAACTG | |
| | GATCTCAACAGCGGTAAGATCCTTGAGAGTTTTCGCCCCG | |
| | AAGAACGTTTTCCAATGATGAGCACTTTTAAAGTTCTGCT | |
| | ATGTGGCGCGGTATTATCCCGTATTGACGCCGGGCAAGA | |
| | GCAACTCGGTCGCCGCATACACTATTCTCAGAATGACTTG | |
| | GTTGAGTACTCACCAGTCACAGAAAAGCATCTTACGGAT | |
| | GGCATGACAGTAAGAGAATTATGCAGTGCTGCCATAACC | |
| | ATGAGTGATAACACTGCGGCCAACTTACTTCTGACAACG | |
| | ATCGGAGGACCGAAGGAGCTAACCGCTTTTTTGCACAAC | |
| | ATGGGGGATCATGTAACTCGCCTTGATCGTTGGGAACCG | |
| | GAGCTGAATGAAGCCAT | |
| 34 | ATGGAGGAGCCGCAGTCAGATCCTAGCGTCGAGCCCCCT | TP53 |
| | CTGAGTCAGGAAACATTTTCAGACCTATGGAAACTACTTC | |
| | CTGAAAACAACGTTCTGTCCCCCTTGCCGTCCCAAGCAAT | |
| | GGATGATTTGATGCTGTCCCCGGACGATATTGAACAATG | |
| | GTTCACTGAAGACCCAGGTCCAGATGAAGCTCCCAGAAT | |
| | GCCAGAGGCTGCTCCCCCCGTGGCCCCTGCACCAGCAGC | |
| | TCCTACACCGGCGGCCCCTGCACCAGCCCCCTCCTGGCCC | |
| | CTGTCATCTTCTGTCCCTTCCCAGAAAACCTACCAGGGCA | |
| | GCTACGGTTTCCGTCTGGGCTTCTTGCATTCTGGGACAGC | |
| | CAAGTCTGTGACTTGCACGTACTCCCCTGCCCTCAACAAG | |
| | ATGTTTTGCCAACTGGCCAAGACCTGCCCTGTGCAGCTGT | |
| | GGGTTGATTCCACACCCCCGCCCGGCACCCGCGTCCGCG | |
| | CCATGGCCATCTACAAGCAGTCACAGCACATGACGGAGG | |
| | TTGTGAGGCGCTGCCCCCACCATGAGCGCTGCTCAGATA | |
| | GCGATGGTCTGGCCCCTCCTCAGCATCTTATCCGAGTGGA | |
| | AGGAAATTTGCGTGTGGAGTATTTGGATGACAGAAACAC | |
| | TTTTCGACATAGTGTGGTGGTGCCCTATGAGCCGCCTGAG | |
| | GTTGGCTCTGACTGTACCACCATCCACTACAACTACATGT | |
| | GTAACAGTTCCTGCATGGGCGGCATGAACCGGAGGCCCA | |
| | TCCTCACCATCATCACACTGGAAGACTCCAGTGGTAATCT | |
| | ACTGGGACGGAACAGCTTTGAGGTGCGTGTTTGTGCCTGT | |
| | CCTGGGAGAGACCGGCGCACAGAGGAAGAGAATCTCCGC | |
| | AAGAAAGGGGAGCCTCACCACGAGCTGCCCCCAGGGAGC | |
| | ACTAAGCGAGCACTGCCCAACAACACCAGCTCCTCTCCC | |
| | CAGCCAAAGAAGAAACCACTGGATGGAGAATATTTCACC | |
| | CTTCAGATCCGTGGGCGTGAGCGCTTCGAGATGTTCCGAG | |
| | AGCTGAATGAGGCCTTGGAACTCAAGGATGCCCAGGCTG | |
| | GGAAGGAGCCAGGGGGGAGCAGGGCTCACTCCAGCCAC | |
| | CTGAAGTCCAAAAAGGGTCAGTCTACCTCCCGCCATAAA | |
| | AAACTCATGTTCAAGACAGAAGGGCCTGACTCAGACTGA | |
| 35 | ATGAAGCTACTGTCTTCTATCGAACAAGCATGCGATATTT | Gal4 DNA- |
| | GCCGACTTAAAAAGCTCAAGTGCTCCAAAGAAAAACCGA | Binding |
| | AGTGCGCCAAGTGTCTGAAGAACAACTGGGAGTGTCGCT | Domain (1- |
| | ACTCTCCCAAAACCAAAAGGTCTCCGCTGACTAGGGCAC | 100)- |
| | ATCTGACAGAAGTGGAATCAAGGCTAGAAAGACTGGAAC | BRCA1 |
| | AGCTATTTCTACTGATTTTTCCTCGAGAAGACCTTGACAT | (1396-1863) |
| | GATTTTGAAAATGGATTCTTTACAGGATATAAAAGCATTG | |
| | TTAACAGGATTATTTGTACAAGATCAGAGGGATACCATG | |
| | CAACATAACCTGATAAAGCTCCAGCAGGAAATGGCTGAA | |
| | CTAGAAGCTGTGTTAGAACAGCATGGGAGCCAGCCTTCT | |
| | AACAGCTACCCTTCCATCATAAGTGACTCCTCTGCCCTTG | |
| | AGGACCTGCGAAATCCAGAACAAAGCACATCAGAAAAA | |
| | GCAGTATTAACTTCACAGAAAAGTAGTGAATACCCTATA | |
| | AGCCAGAATCCAGAAGGCCTTTCTGCTGACAAGTTTGAG | |
| | GTGTCTGCAGATAGTTCTACCAGTAAAAATAAAGAACCA | |
| | GGAGTGGAAAGGTCATCCCCTTCTAAATGCCCATCATTAG | |
| | ATGATAGGTGGTACATGCACAGTTGCTCTGGGAGTCTTCA | |
| | GAATAGAAACTACCCATCTCAAGAGGAGCTCATTAAGGT | |
| | TGTTGATGTGGAGGAGCAACAGCTGGAAGAGTCTGGGCC | |
| | ACACGATTTGACGGAAACATCTTACTTGCCAAGGCAAGA | |
| | TCTAGAGGGAACCCCTTACCTGGAATCTGGAATCAGCCTC | |
| | TTCTCTGATGACCCTGAATCTGATCCTTCTGAAGACAGAG | |
| | CCCCAGAGTCAGCTCGTGTTGGCAACATACCATCTTCAAC | |
| | CTCTGCATTGAAAGTTCCCCAATTGAAAGTTGCAGAATCT | |
| | GCCCAGGGTCCAGCTGCTGCTCATACTACTGATACTGCTG | |
| | GGTATAATGCAATGGAAGAAAGTGTGAGCAGGGAGAAG | |
| | CCAGAATTGACAGCTTCAACAGAAAGGGTCAACAAAAGA | |
| | ATGTCCATGGTGGTGTCTGGCCTGACCCCAGAAGAATTTA | |
| | TGCTCGTGTACAAGTTTGCCAGAAAACACCACATCACTTT | |
| | AACTAATCTAATTACTGAAGAGACTACTCATGTTGTTATG | |
| | AAAACAGATGCTGAGTTTGTGTGTGAACGGACACTGAAA | |
| | TATTTTCTAGGAATTGCGGGAGGAAAATGGGTAGTTAGC | |
| | TATTTCTGGGTGACCCAGTCTATTAAAGAAAGAAAAATG | |
| | CTGAATGAGCATGATTTTGAAGTCAGAGGAGATGTGGTC | |
| | AATGGAAGAAACCACCAAGGTCCAAAGCGAGCAAGAGA | |
| | ATCCCAGGACAGAAAGATCTTCAGGGGGCTAGAAATCTG | |
| | TTGCTATGGGCCCTTCACCAACATGCCCACAGATCAACTG | |
| | GAATGGATGGTACAGCTGTGTGGTGCTTCTGTGGTGAAG | |
| | GAGCTTTCATCATTCACCCTTGGCACAGGTGTCCACCCAA | |
| | TTGTGGTTGTGCAGCCAGATGCCTGGACAGAGGACAATG | |
| | GCTTCCATGCAATTGGGCAGATGTGTGAGGCACCTGTGGT | |
| | GACCCGAGAGTGGGTGTTGGACAGTGTAGCACTCTACCA | |
| | GTGCCAGGAGCTGGACACCTACCTGATACCCCAGATCCC | |
| | CCACAGCCACTACTGA | |

I claim:

1. A suppressor tRNA molecule comprising a mutant Methanomethylophilus alvus pyrrolysyl tRNA molecule, wherein the mutant M. alvus pyrrolysyl tRNA molecule further comprises:
a) a mutated acceptor stem comprising a human tRNA identity element; and
b) an anticodon loop comprising three nucleotide residues which are complementary to a premature termination codon on an mRNA template encoding a polypeptide.

2. The suppressor tRNA molecule of claim 1, wherein the mutated acceptor stem comprises a region of unpaired nucleotides comprising four nucleotides on a single strand and a region of base-paired nucleotides comprising fourteen paired of nucleotides; wherein:
a) the region of unpaired nucleotides comprises at least one but less than four point mutations; and
b) the region of base-paired nucleotides comprises at least one but less than seven point mutations.

3. The suppressor tRNA molecule of claim 2, wherein:
a) the region of unpaired nucleotides comprises a guanine to adenine point mutation; and
b) the region of base-paired nucleotides comprises at least one but less than four uracil to cytosine point mutation(s) and/or at least one but less than four cytosine to uracil point mutation(s).

4. The suppressor tRNA molecule of claim 2, wherein:
a) the region of unpaired nucleotides comprises a single guanine to adenine point mutation; and
b) the region of base-paired nucleotides comprises one or two uracil to cytosine point mutation(s) and one cytosine to uracil point mutation.

5. The suppressor tRNA molecule of claim 1, comprising the mutations C66U/U67C/G69A, C29U/C66U/U67C/G69A, C29U/U30C/C66U/U67C/G69A, U65C/C66U/U67C/G69A, C29U/U65C/C66U/U67C/G69A, or C29U/U30C/U65C/C66U/U67C/G69A with reference to SEQ ID NO:1.

6. The suppressor tRNA molecule of claim 1, wherein the anticodon loop comprises the nucleotide sequence: CUA, UUA or UCA.

7. The suppressor tRNA molecule of claim 1, wherein the suppressor tRNA molecule is aminoacylated by a human aminoacyl-tRNA synthetase.

8. The suppressor tRNA molecule of claim 7, wherein the human aminoacyl-tRNA synthetase is human alanyl-tRNA synthetase and the suppressor tRNA molecule is aminoacylated with alanine.

9. The suppressor tRNA molecule of claim 1, comprising an RNA sequence having at least about 85%, 90%, 95%, 98%, or 99% sequence identity to SEQ ID NO:2, SEQ ID NO:3, SEQ ID NO:4, SEQ ID NO:5, SEQ ID NO:6, or SEQ ID NO:7.

10. A polynucleotide sequence encoding the suppressor tRNA molecule of claim 1.

11. An expression system comprising an expression cassette comprising a polynucleotide sequence encoding a suppressor tRNA molecule,
wherein the polynucleotide sequence encoding the suppressor tRNA molecule comprises any one of SEQ ID NO: 9, SEQ ID NO:10, SEQ ID NO:11, SEQ ID NO: 12, SEQ ID NO: 13, and SEQ ID NO:14, or the complement thereof, or a polynucleotide sequence having at least about 85%, 90%, 95%, 98%, or 99% sequence identity to SEQ ID NO: 9, SEQ ID NO: 10, SEQ ID NO: 11, SEQ ID NO: 12, SEQ ID NO:13, and SEQ ID NO:14, or the complement thereof; and
wherein the polynucleotide sequence encoding the suppressor tRNA molecule is operably linked to a promoter.

12. The expression system of claim 11, wherein the expression cassette is stably integrated into the genome of a host cell.

13. The expression system of claim 11, wherein the expression cassette comprises at least one copy of the polynucleotide sequence encoding the suppressor tRNA molecule.

14. The expression system of claim 11, wherein the expression cassette comprises multiple copies of the polynucleotide sequence encoding the suppressor tRNA molecule, wherein the multiple copies of the polynucleotide sequence encoding the suppressor tRNA molecule are repeated in tandem.

15. The expression system of claim 11, wherein the promoter is an RNA polymerase type III promoter or a derivative thereof.

16. The expression system of claim 15, wherein the RNA polymerase type III promoter or the derivative thereof is of mammalian origin.

17. The expression system of claim 16, wherein the RNA polymerase type III promoter is a U6 promoter, H1 promoter, 7SK promoter, 7SL promoter, Y3 promoter, 5S rRNA promoter, Ad2 VAI, VAII promoter, or a derivative thereof.

18. The expression system of claim 17, wherein the RNA polymerase type III promoter is a human U6 promoter, a human H1 promoter, a human 7SK promoter, or a derivative thereof.

19. The expression system of claim 17, wherein the RNA polymerase type III promoter comprises a polynucleotide sequence having at least about 80%, 85%, 90%, 95%, 98%, 99% sequence identity to SEQ ID NO:15, SEQ ID NO:16, or SEQ ID NO:17.

20. The expression system of claim 11, wherein the expression cassette further comprises a terminator sequence, wherein the terminator sequence flanks the 3′ end of the polynucleotide sequence encoding the suppressor tRNA molecule.

21. The expression system of claim 20, wherein the terminator sequence comprises a nucleotide sequence set forth in SEQ ID NO:18, SEQ ID NO: 19, SEQ ID NO:20, SEQ ID NO: 21, SEQ ID NO:22, SEQ ID NO:23, SEQ ID NO:24, or SEQ ID NO:25.

22. The expression system of claim 11, wherein the expression cassette comprises SEQ ID NO: 30, or a polynucleotide sequence having at least about 75%, 80%, 85%, 90%, 95%, 98%, or 99% sequence identity to SEQ ID NO:30.

23. The expression system of claim 11, further comprising a viral vector.

24. The expression system of claim 23, wherein the viral vector is an adenoviral vector, a retroviral vector, or a simian virus 40 (SV40) viral vector.

25. The expression system of claim 12, wherein the host cell is a bacterial cell or a mammalian cell.

26. The expression system of claim 25, wherein the host cell is a human cell.

27. A method of treating a genetic disease in a subject comprising administering to the subject the suppressor tRNA molecule of claim 1,
wherein the genetic disease is characterized by the presence of a premature termination codon in an mRNA template encoding a polypeptide, and
wherein the premature termination codon replaces a sense codon in the mRNA template in the subject.

28. The method of claim 27, wherein the disease is further characterized by the presence of a truncated polypeptide or the absence of the full-length polypeptide.

29. The method of claim 28, wherein the premature termination codon comprises the stop codon UAG, UAA, or UGA.

30. The method of claim 27, wherein the suppressor tRNA molecule comprises the nucleotide sequence:
a) CUA, wherein three nucleotide residues base pair with the stop codon UAG;
b) UUA, wherein the three nucleotide residues base pair with the stop codon UAA; or
c) UCA, wherein the three nucleotide residues base pair with the stop codon UGA.

31. The method of claim 27, wherein the genetic disease is a cancer.

32. The method of claim 31, wherein the cancer is colorectal cancer, breast cancer, or ovarian cancer.


SEQUENCE LISTING (35 sequences)

SEQ ID NO: 1 - RNA, 69 nt, Methanomethylophilus alvus
  source 1..69: mol_type = tRNA
gggggacggtccggcgaccagcgggtctctaaaacctagcatagcggggttcgacaccccggtctctcg

SEQ ID NO: 2 - RNA, 69 nt, synthetic construct
  source 1..69: mol_type = other RNA
gggggacggtccggcgaccagcgggtctctaaaacctagcatagcggggttcgacaccccggtcttcca

SEQ ID NO: 3 - RNA, 69 nt, synthetic construct
  source 1..69: mol_type = other RNA
gggggacggtccggcgaccagcgggtctttaaaacctagcatagcggggttcgacaccccggtcttcca

SEQ ID NO: 4 - RNA, 69 nt, synthetic construct
  source 1..69: mol_type = other RNA
gggggacggtccggcgaccagcgggtcttcaaaacctagcatagcggggttcgacaccccggtcttcca

SEQ ID NO: 5 - RNA, 69 nt, synthetic construct
  source 1..69: mol_type = other RNA
gggggacggtccggcgaccagcgggtctctaaaacctagcatagcggggttcgacaccccggtcctcca

SEQ ID NO: 6 - RNA, 69 nt, synthetic construct
  source 1..69: mol_type = other RNA
gggggacggtccggcgaccagcgggtctttaaaacctagcatagcggggttcgacaccccggtcctcca

SEQ ID NO: 7 - RNA, 69 nt, synthetic construct
  source 1..69: mol_type = other RNA
gggggacggtccggcgaccagcgggtcttcaaaacctagcatagcggggttcgacaccccggtcctcca

SEQ ID NO: 8 - RNA, 69 nt, Methanomethylophilus alvus; identical to SEQ ID NO: 1
  source 1..69: mol_type = tRNA

SEQ ID NO: 9 - RNA, 69 nt, synthetic construct; identical to SEQ ID NO: 2
  source 1..69: mol_type = other RNA

SEQ ID NO: 10 - RNA, 69 nt, synthetic construct; identical to SEQ ID NO: 3
  source 1..69: mol_type = other RNA

SEQ ID NO: 11 - RNA, 69 nt, synthetic construct; identical to SEQ ID NO: 4
  source 1..69: mol_type = other RNA

SEQ ID NO: 12 - RNA, 69 nt, synthetic construct; identical to SEQ ID NO: 5
  source 1..69: mol_type = other RNA

SEQ ID NO: 13 - RNA, 69 nt, synthetic construct; identical to SEQ ID NO: 6
  source 1..69: mol_type = other RNA

SEQ ID NO: 14 - RNA, 69 nt, synthetic construct; identical to SEQ ID NO: 7
  source 1..69: mol_type = other RNA

SEQ ID NO: 15 - DNA, 243 bp, Homo sapiens
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 16 - DNA, 249 bp, Homo sapiens
  source 1..249: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 17 - DNA, 223 bp, Homo sapiens
  source 1..223: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccc

SEQ ID NO: 18-22 - skipped (5 entries)

SEQ ID NO: 23 - DNA, 10 bp, synthetic construct
  source 1..10: mol_type = other DNA
tttttttttt

SEQ ID NO: 24 - DNA, 11 bp, synthetic construct
  source 1..11: mol_type = other DNA
ttttttttttt

SEQ ID NO: 25 - DNA, 12 bp, synthetic construct
  source 1..12: mol_type = other DNA
tttttttttttt

SEQ ID NO: 26 - DNA, 204 bp, Cauliflower mosaic virus
  source 1..204: mol_type = other DNA
agctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcac

SEQ ID NO: 27 - DNA, 380 bp, Cauliflower mosaic virus
  source 1..380: mol_type = other DNA
catggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtc

SEQ ID NO: 28 - DNA, 726 bp, synthetic construct
  source 1..726: mol_type = other DNA
tcagctgcccttgtacagctcgtccatgccgtgggtgatgccagcggcggtcacgaattccagcagcaccatgtggtcccgcttctcgttggggtccttgctcagcacgctctgggtgctcaggtagtggttgtcgggcagcagcacggggccatctccgatgggggtgttctgctggtagtggtcggccagctgcacgctgccatcttccacgttgtgccggatcttgaagttggccttgatgccgttcttctgcttgtcggcggtgatgtacacctagtggctgttgaagttgtactccagcttgtggcccaggatgttgccgtcctctttgaaatcgatgcccttcagctcgatccggttcacgagggtgtcgccctcgaacttcacttcggctctggtcttgtaggtgccgtcgtccttgaagctgatggtccgttcctgcacgtagccctcgggcatggcgctcttgaagaagtcgtgccgcttcatatggtcggggtatctgctgaagcactgcacgccgtaggtcagtgtggtcacgagggtaggccaaggcacgggcagcttgccggtggtgcagatgaactccagggtcagcttgccatttgtggcgtcgccttcgccctctccccgcacagagaacttgtggccgttcacgtcgccatccagttccaccaggatgggcaccacgccggtgaacagttcctcgcccttggacaccat

SEQ ID NO: 29 - DNA, 225 bp, synthetic construct
  source 1..225: mol_type = other DNA
ccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 30 - DNA, 990 bp, synthetic construct
  source 1..990: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttgctaatccgtatcgctacctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttacctgcatggtactcgtactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccatttttt

SEQ ID NO: 31 - DNA, 7842 bp, synthetic construct
  source 1..7842: mol_type = other DNA
agtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttggtcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagccccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagtcatcagctgcccttgtacagctcgtccatgccgtgggtgatgccagcggcggtcacgaattccagcagcaccatgtggtcccgcttctcgttggggtccttgctcagcacgctctgggtgctcaggtagtggttgtcgggcagcagcacggggccatctccgatgggggtgttctgctggtagtggtcggccagctgcacgctgccatcttccacgttgtgccggatcttgaagttggccttgatgccgttcttctgcttgtcggcggtgatgtacacctagtggctgttgaagttgtactccagcttgtggcccaggatgttgccgtcctctttgaaatcgatgcccttcagctcgatccggttcacgagggtgtcgccctcgaacttcacttcggctctggtcttgtaggtgccgtcgtccttgaagctgatggtccgttcctgcacgtagccctcgggcatggcgctcttgaagaagtcgtgccgcttcatatggtcggggtatctgctgaagcactgcacgccgtaggtcagtgtggtcacgagggtaggccaaggcacgggcagcttgccggtggtgcagatgaactccagggtcagcttgccatttgtggcgtcgccttcgccctctccccgcacagagaacttgtggccgttcacgtcgccatccagttccaccaggatgggcaccacgccggtgaacagttcctcgcccttggacaccatggtggcgaattccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctcctcgcagatcctttctagcatttgacaggcacattatgctggcgcctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttgctaatccgtatcgctacctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttacctgcatggtactcgtactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttggatcctctagagtcgacctgcagatccttagactagttggccgtggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctcataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacg

SEQ ID NO: 32 - DNA, 1521 bp, synthetic construct
  source 1..1521: mol_type = other DNA
tcaacctgggtgcgctgcgtctgtggttcctgcgggcggtgcaaggtcacaggcggtgtatggttcgaggcacgctgcgaaagccattacatgggctatgaaagtctgttcttgcacaccatgcacaaggtgtgcctgggggccctgagcgaaaacagcgacatcttcgccagcatgcgtttcctcatccagtgggactgcgctttgctgccggtattctggtgagcctgactctgattcagttacatctggtctagctccgtccttgagtacgtagcccggtccgtttccatacaacaacaccgtgtaggctttccgatcgcgcgcttttcccggtgcaagcccgaaaatactactgcctcgcaacgggtaccctccaaatgagaaaacatggctgtgatctgcggttacgagagaaagtgtgtcctcttcagatgtcaattgtcccgctcgctctattgcgtcatcaaacataattgtctctgtcagcgcgcgataggcccgagactcatgatgtccatgatctatcctaccgccctcaacgaaaaggaagaaccctctagggtttctggaaagaagtcgcaaagcagcctccgtcatctccataagagaagggtcaagcgtactgtcgcggtgtatctcgtacttcatgtcgccaggttcgaaaagccccatcagatgggtgactgacggatccaggctagcttgcatcaattcagttctattccatacatatcgcgcaccttgtctcttagccagccattcttggaccaggttcttaccatccaagcgggtgcctccttgggagtagtcatccgggtattctgggtcaggagtccccattcggaacatgtactttctcccaccacccaggatgacatctatatccatattggagatcaactgcgtcgcgatgtcctggcacccttcttgccttgcggaggccgggacatctgcgtcagaataccagtttctgttcacggtatgggcatacgtcccggccggggacgcgtgttgcaccctagtagtcgtcaccaccccgacacttttacccgctttctttgcccggttcatgacgctaataacctcgttaccgcgtgtggtattacattggttaaaccgagcggcagcagaaagtccgatagtttgaaagtttcctttcactccacagaggtacgcagttgcagttgcaccggaatcaggtacgtgtttatccacgttgtaggttttgctaagggccacataggggaagcggtccattgcaagcggtatttcaggtcccagtttgtcttttttctgacctttcagtattcttgctgctgtgacggtggagacgcccatgccatctcccagaaaaataataaggtttttcgccgctgtttgtgctggctgcagcttctacgcagctccgagagcctctgctgcttctctattccaaaaatcgggattttcttcttctacgggtattatccccagggacagctgaagtcgcaggccaagcaacaagaggagaagcaacat

SEQ ID NO: 33 - DNA, 8634 bp, synthetic construct
  source 1..8634: mol_type = other DNA
accaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttggtcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagccccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagtcaacctgggtgcgctgcgtctgtggttcctgcgggcggtgcaaggtcacaggcggtgtatggttcgaggcacgctgcgaaagccattacatgggctatgaaagtctgttcttgcacaccatgcacaaggtgtgcctgggggccctgagcgaaaacagcgacatcttcgccagcatgcgtttcctcatccagtgggactgcgctttgctgccggtattctggtgagcctgactctgattcagttacatctggtctagctccgtccttgagtacgtagcccggtccgtttccatacaacaacaccgtgtaggctttccgatcgcgcgcttttcccggtgcaagcccgaaaatactactgcctcgcaacgggtaccctccaaatgagaaaacatggctgtgatctgcggttacgagagaaagtgtgtcctcttcagatgtcaattgtcccgctcgctctattgcgtcatcaaacataattgtctctgtcagcgcgcgataggcccgagactcatgatgtccatgatctatcctaccgccctcaacgaaaaggaagaaccctctagggtttctggaaagaagtcgcaaagcagcctccgtcatctccataagagaagggtcaagcgtactgtcgcggtgtatctcgtacttcatgtcgccaggttcgaaaagccccatcagatgggtgactgacggatccaggctagcttgcatcaattcagttctattccatacatatcgcgcaccttgtctcttagccagccattcttggaccaggttcttaccatccaagcgggtgcctccttgggagtagtcatccgggtattctgggtcaggagtccccattcggaacatgtactttctcccaccacccaggatgacatctatatccatattggagatcaactgcgtcgcgatgtcctggcacccttcttgccttgcggaggccgggacatctgcgtcagaataccagtttctgttcacggtatgggcatacgtcccggccggggacgcgtgttgcaccctagtagtcgtcaccaccccgacacttttacccgctttctttgcccggttcatgacgctaataacctcgttaccgcgtgtggtattacattggttaaaccgagcggcagcagaaagtccgatagtttgaaagtttcctttcactccacagaggtacgcagttgcagttgcaccggaatcaggtacgtgtttatccacgttgtaggttttgctaagggccacataggggaagcggtccattgcaagcggtatttcaggtcccagtttgtcttttttctgacctttcagtattcttgctgctgtgacggtggagacgcccatgccatctcccagaaaaataataaggtttttcgccgctgtttgtgctggctgcagcttctacgcagctccgagagcctctgctgcttctctattccaaaaatcgggattttcttcttctacgggtattatccccagggacagctgaagtcgcaggccaagcaacaagaggagaagcaacatggtggcgaattccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctcctcgcagatcctttctagcatttgacaggcacattatgctggcgcctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttgctaatccgtatcgctacctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttacctgcatggtactcgtactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcgggggacggtccggcgaccagcgggtctctaaaacctagcatagcggggttcgacaccccggtcttccattttttggatcctctagagtcgacctgcagatccttagactagttggccgtggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctcataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccat

SEQ ID NO: 34 - DNA, 1182 bp, Homo sapiens
  source 1..1182: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 35 - DNA, 1707 bp, Homo sapiens
  source 1..1707: mol_type = genomic DNA
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagatcagagggataccatgcaacataacctgataaagctccagcaggaaatggctgaactagaagctgtgttagaacagcatgggagccagccttctaacagctacccttccatcataagtgactcctctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagggtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga